(19) United States
(12) Patent Application Publication
(10) Pub. No.: US 2024/0190893 A1
(43) Pub. Date: Jun. 13, 2024

(54) MACROCYCLIC 1,3-BRIDGED 6-CHLORO-7-PYRAZOL-4-YL-1 H-INDOLE-2-CARBOXYLATE AND 6-CHLORO-7-PYRIMIDIN-5-YL-1H-INDOLE-2-CARBOXYLATE DERIVATIVES AS MCL-1 INHIBITORS FOR THE TREATMENT OF CANCER

(71) Applicant: JANSSEN PHARMACEUTICA NV, Beerse (BE)

(72) Inventors: Benoît Christian Albert Ghislain DE BOECK, Genval (BE); Frederik Jan Rita ROMBOUTS, Wilrijk (BE); Soufyan JERHAOUI, Turnhout (BE)

(21) Appl. No.: 18/546,116

(22) PCT Filed: Feb. 11, 2022
(86) PCT No.: PCT/EP2022/053340

§ 371 Date: Aug. 11, 2023

(30) Foreign Application Priority Data
Feb. 12, 2021 (EP) 21156777.1

(51) Int. Cl. C07D 515/18; A61K 31/506; A61P 35/02

(52) U.S. Cl. CPC C07D 515/18; A61K 31/506; A61P 35/02

(57) ABSTRACT

The present invention relates to compounds of formula (I) wherein XI is as myeloid cell leukemia-1 (MCL-1) inhibitors for the treatment of cancer, such as e.g. prostate, lung, pancreatic, breast, ovarian, and cervical cancer, melanoma, B-cell chronic lymphocytic leukemia (CLL), acute myeloid leukemia (AML), and acute lymphoblastic leukemia (ALL). An exemplary compound is e.g. compound 1

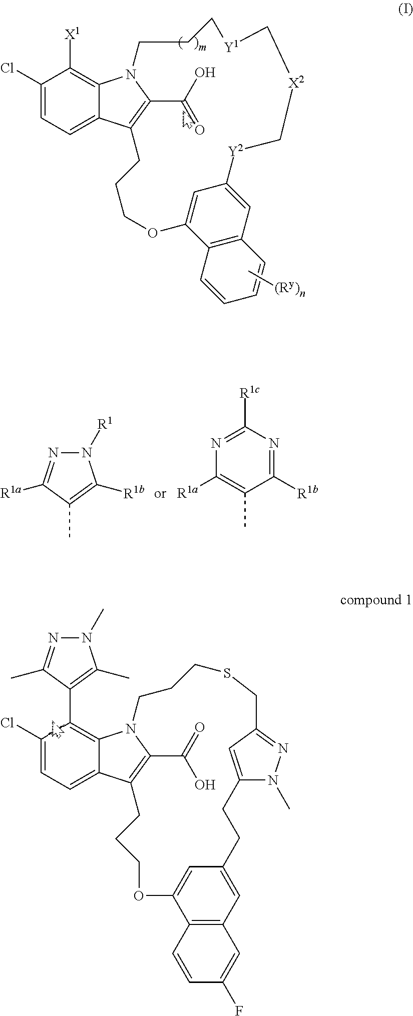

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009; (113) 2805-2815).

WO2018178226 discloses MCL-1 inhibitors and methods of use thereof.

WO2017182625 discloses macrocyclic MCL-1 inhibitors for treating cancer.

WO2018178227 discloses the synthesis of MCL-1 inhibitors.

WO2020063792 discloses indole macrocyclic derivatives.

CN110845520 discloses macrocyclic indoles as MCL-1 inhibitors.

WO2020103864 discloses macrocyclic indoles as MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns novel compounds of Formula (I):

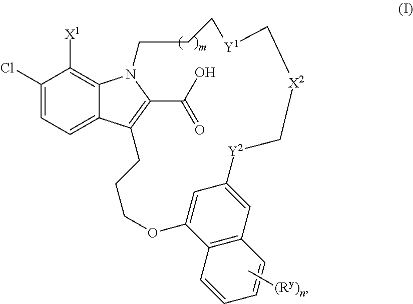

and the tautomers and the stereoisomeric forms thereof, wherein
X¹ represents

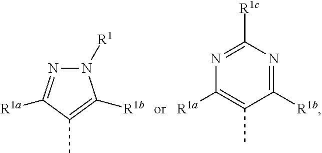

wherein
R¹, R^{1a}, R^{1b}, R^{1c}, and R² each independently represent hydrogen; or C₁₋₆alkyl optionally substituted with one or two substituents each independently selected from the group consisting of Het¹, —OR₃, and —NR^{4a}R^{4b}; Het¹ represents morpholinyl or tetrahydropyranyl;
R³ represents hydrogen, C₁₋₄alkyl, —C₂₋₄alkyl-O—C₁₋₄alkyl, —C₂₋₄alkyl-OH, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents

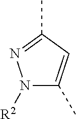

which can be attached to the remainder of the molecule in both directions;
Y¹ represents —O—, —S—, —S(═O)—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
Y² represents —CH₂—, —S— or —S(═O)₂—;
R^{y} represents halo;
n represents 0, 1 or 2;
m represents 0 or 1;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘Cy’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₄alkyl’ as used herein as a group or part of a group represents a straight or branched chain fully saturated hydrocarbon radical having from 2 to 4 carbon atoms, such as ethyl, n-propyl, isopropyl, n-butyl, s-butyl, t-butyl and the like.

The term ‘C₂₋₆alkyl’ as used herein as a group or part of a group represents a straight or branched chain fully saturated hydrocarbon radical having from 2 to 6 carbon atoms, such as ethyl, n-propyl, isopropyl, n-butyl, s-butyl, t-butyl, n-pentyl, n-hexyl and the like.

The term ‘C₃₋₆cycloalkyl’ as used herein as a group or part of a group defines a fully saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

It will be clear that a Compound of Formula (I) includes Compounds of Formula (I-x) and (I-y) (both directions of X² being

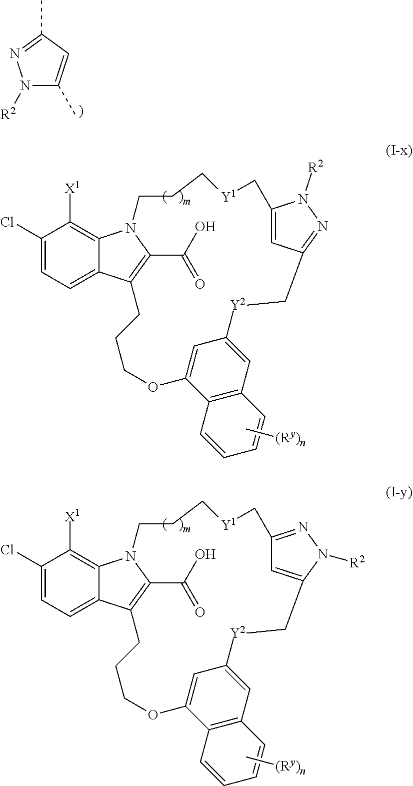

When any variable occurs more than one time in any constituent or in any formula (e.g. Formula (I)), each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

In particular, the compounds disclosed herein possess axial chirality, by virtue of restricted rotation around a biaryl bond and as such may exist as mixtures of atropisomers. When a compound is a pure atropisomer, the stereochemistry at each chiral center may be specified by either Rₐ or Sₐ. Such designations may also be used for mixtures that are enriched in one atropisomer. Further description of atropisomerism and axial chirality and rules for assignment of configuration can be found in Eliel, E. L. & Wilen, S. H. ‘Stereochemistry of Organic Compounds’ John Wiley and Sons, Inc. 1994.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light. Optically active (Rₐ)- and (Sₐ)-atropisomers may be prepared using chiral synthons, chiral reagents or chiral catalysts, or resolved using conventional techniques well known in the art, such as chiral HPLC.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer; when a compound of Formula (I) is for instance specified as Rₐ, this means that the compound is substantially free of the Sₐ atropisomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein
X¹ represents

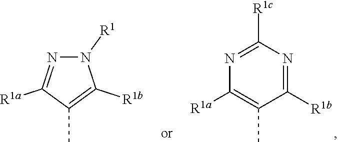

wherein
R¹, R^{1a}, R^{1b}, R^{1c}, and R² each independently represent hydrogen; methyl; or C₂₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, —C₂₋₄alkyl-OH, or —C₂₋₄alkyl-O—C₂₋₄alkyl-O—C₁₋₄alkyl; R^{4a} and R^{4b} are each independently selected from the group consisting of hydrogen and C₁₋₄alkyl;
X² represents

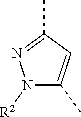

which can be attached to the remainder of the molecule in both directions;
Y¹ represents —O—, —S—, —S(═O)—, —S(═O)₂—, or —N(R^{x})—; R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
Y² represents —CH₂—, —S— or —S(═O)₂—;
R^{y} represents halo;
n represents 0, 1 or 2;
m represents 0 or 1;

The present invention relates in particular to compounds of Formula (I) as defined herein, and the tautomers and the stereoisomeric forms thereof, wherein
X¹ represents

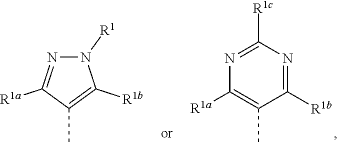

wherein
R¹, R^{1a}, R^{1b}, R^{1c}, and R² each independently represent hydrogen; or C₁₋₆alkyl optionally substituted with one —OR³;
R³ represents hydrogen, C₁₋₄alkyl, or —C₂₋₄alkyl-O—C₁₋₄alkyl;
X² represents

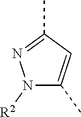

which can be attached to the remainder of the molecule in both directions;
Y¹ represents —S—, —S(═O)—, or —S(═O)₂—;
Y² represents —CH₂— or —S—;
R^{y} represents halo;
n represents 0 or 1;
m represents 0 or 1;
and the pharmaceutically acceptable salts and the solvates thereof.

The present invention relates in particular to compounds of Formula (I) as defined herein, and the tautomers and the stereoisomeric forms thereof, wherein
X¹ represents

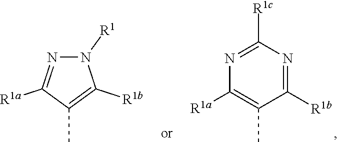

wherein
R¹, R^{1a}, R^{1b}, R^{1c}, and R² each independently represent hydrogen; methyl, or C₂₋₆alkyl optionally substituted with one —OR³;
R³ represents hydrogen, C₁₋₄alkyl, or —C₂₋₄alkyl-O—C₁₋₄alkyl;
X² represents

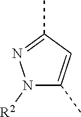

which can be attached to the remainder of the molecule in both directions;
Y¹ represents —S—, —S(═O)—, or —S(═O)₂—;
Y² represents —CH₂— or —S—;
R^{y} represents halo;
n represents 0 or 1;
m represents 0 or 1;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein
n represents 1; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein R¹ represents hydrogen.

In an embodiment, the present invention relates to those compounds of Formula (I) and the pharmaceutically acceptable salts, and the solvates thereof, or any subgroup thereof as mentioned in any of the other embodiments, wherein R¹ represents C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R¹ represents methyl.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein n represents 0.

In an embodiment, the present invention relates to those compounds of Formula (I) and the pharmaceutically acceptable salts, and the solvates thereof, or any subgroup thereof as mentioned in any of the other embodiments, wherein n represents 1.

In an embodiment, the present invention relates to those compounds of Formula (I) and the pharmaceutically acceptable salts, and the solvates thereof, or any subgroup thereof as mentioned in any of the other embodiments, wherein m represents 0.

In an embodiment, the present invention relates to those compounds of Formula (I) and the pharmaceutically acceptable salts, and the solvates thereof, or any subgroup thereof as mentioned in any of the other embodiments, wherein m represents 1.

In an embodiment, the present invention relates to those compounds of Formula (I) and the pharmaceutically acceptable salts, and the solvates thereof, or any subgroup thereof as mentioned in any of the other embodiments, wherein R³ represents hydrogen, C₁₋₄alkyl, or-C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein
Y¹ represents —S—, —S(═O)—, or —S(═O)₂—.

In an embodiment, the present invention relates to those compounds of Formula (I) and the pharmaceutically acceptable salts, and the solvates thereof, or any subgroup thereof as mentioned in any of the other embodiments, wherein
Y¹ represents —S—.

In an embodiment, the present invention relates to those compounds of Formula (I) and the pharmaceutically acceptable salts, and the solvates thereof, or any subgroup thereof as mentioned in any of the other embodiments, wherein
Y¹ represents —S(═O)—.

In an embodiment, the present invention relates to those compounds of Formula (I) and the pharmaceutically acceptable salts, and the solvates thereof, or any subgroup thereof as mentioned in any of the other embodiments, wherein
Y¹ represents —S(═O)₂—.

In an embodiment, the present invention relates to those compounds of Formula (I) and the pharmaceutically acceptable salts, and the solvates thereof, or any subgroup thereof as mentioned in any of the other embodiments, wherein
Y² represents —CH₂— or —S—.

In an embodiment, the present invention relates to those compounds of Formula (I) and the pharmaceutically acceptable salts, and the solvates thereof, or any subgroup thereof as mentioned in any of the other embodiments, wherein
Y² represents —CH₂—.

In an embodiment, the present invention relates to those compounds of Formula (I) and the pharmaceutically acceptable salts, and the solvates thereof, or any subgroup thereof as mentioned in any of the other embodiments, wherein
Y² represents —S—.

In an embodiment, the present invention relates to those compounds of Formula (I) and the pharmaceutically acceptable salts, and the solvates thereof, or any subgroup thereof as mentioned in any of the other embodiments, wherein
R¹, R^{1a}, R^{1b}, R^{1c}, and R² each independently represent hydrogen; methyl; or C₂₋₆alkyl optionally substituted with one or two substituents each independently selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein X¹ represents

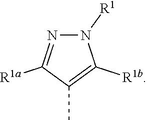

In an embodiment, the present invention relates to those compounds of Formula (I) and the pharmaceutically acceptable salts, and the solvates thereof, or any subgroup thereof as mentioned in any of the other embodiments, wherein X¹ represents

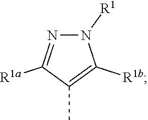

and R¹, R^{1a}, R^{1b}, and R² each independently represent hydrogen; methyl; or C₂₋₆alkyl optionally substituted with one or two substituents each independently selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein X¹ represents

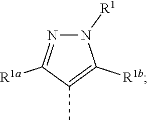

and R¹, R^{1a}, R^{1b}, and R² each independently represent hydrogen; methyl; or C₂₋₆alkyl optionally substituted with one or two substituents each independently selected from the group consisting of Het¹, —OR³, and —NR^{4a}R^{4b}; and wherein at least one of R¹, R^{1a}, and R^{1b} is other than hydrogen.

In an embodiment, the present invention relates to those compounds of Formula (I) and the pharmaceutically acceptable salts, and the solvates thereof, or any subgroup thereof as mentioned in any of the other embodiments, wherein X¹ represents

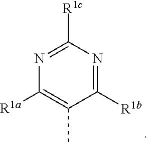

In an embodiment, the present invention relates to those compounds of Formula (I) and the pharmaceutically acceptable salts, and the solvates thereof, or any subgroup thereof as mentioned in any of the other embodiments, wherein X¹ represents

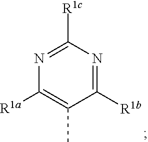

and R^{1a}, R^{1b}, R^{1c} and R² each independently represent hydrogen; methyl; or C₂₋₆alkyl optionally substituted with one or two substituents each independently selected from the group consisting of Het¹, —OR₃, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein X¹ represents

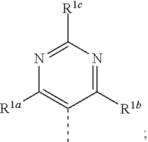

and R^{1a}, R^{1b}, R^{1c} and R² each independently represent hydrogen; methyl; or C₂₋₆alkyl optionally substituted with one or two substituents each independently selected from the group consisting of Het¹, —OR₃, and —NR^{4a}R^{4b}; and wherein at least one of R¹, R^{1b}, and R^{1c} is other than hydrogen.

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below:

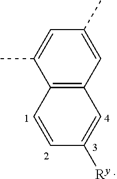

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below; and wherein R^{y} represents fluoro:

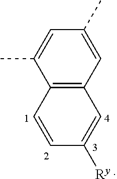

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

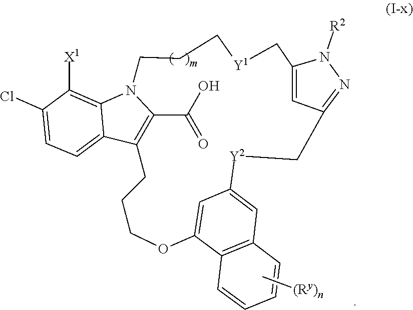

It will be clear that all variables in the structure of Formula (I-x), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein Formula (I) is limited to Formula (I-x) as defined herein, wherein
R¹, R^{1a}, R^{1b}, R^{1c}, and R² each independently represent hydrogen; methyl; or C₂₋₆alkyl optionally substituted with one or two substituents each independently selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

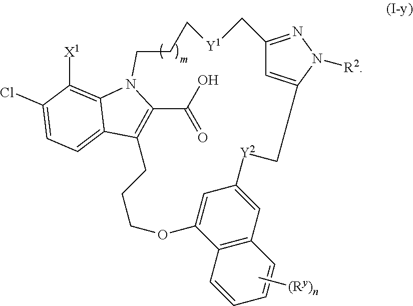

It will be clear that all variables in the structure of Formula (I-y), are defined as defined for the compounds of Formula (I) or any subgroup thereof as mentioned in any of the other embodiments.

The present invention relates in particular to compounds of Formula (I-y) as defined herein, and the tautomers and the stereoisomeric forms thereof, wherein
X¹ represents

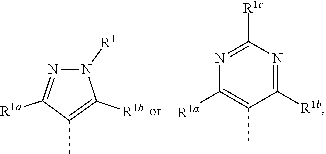

wherein
R¹, R^{1a}, R^{1b}, R^{1c}, and R² each independently represent hydrogen; or C₁₋₆alkyl optionally substituted with one —OR³;
R³ represents hydrogen, C₁₋₄alkyl, or —C₂₋₄alkyl-O—C₁₋₄alkyl;
Y¹ represents —S—, —S(═O)—, or —S(═O)₂—;
Y² represents —CH₂— or —S—;
R^{y} represents halo;
n represents 0 or 1;
m represents 0 or 1;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein Formula (I) is limited to Formula (I-y) as defined herein, wherein
R¹, R^{1a}, R^{1b}, R^{1c}, and R² each independently represent hydrogen; methyl; or C₂₋₆alkyl optionally substituted with one or two substituents each independently selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein the compounds are Rₐ atropisomers.

In an embodiment, the present invention relates to those compounds of Formula (I) and the pharmaceutically acceptable salts, and the solvates thereof, or any subgroup thereof as mentioned in any of the other embodiments, wherein the compounds are Sₐ atropisomers.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof,
any pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or can be prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

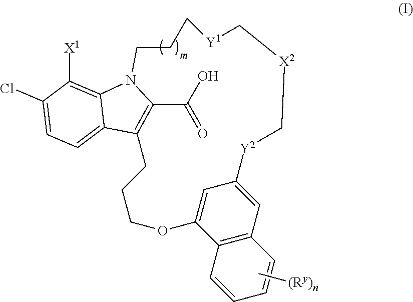

Compounds of Formula (I), wherein X¹, V, Y¹, Y², R^{y}, m, and n are defined as in the general scope, can be prepared according to Scheme 1,

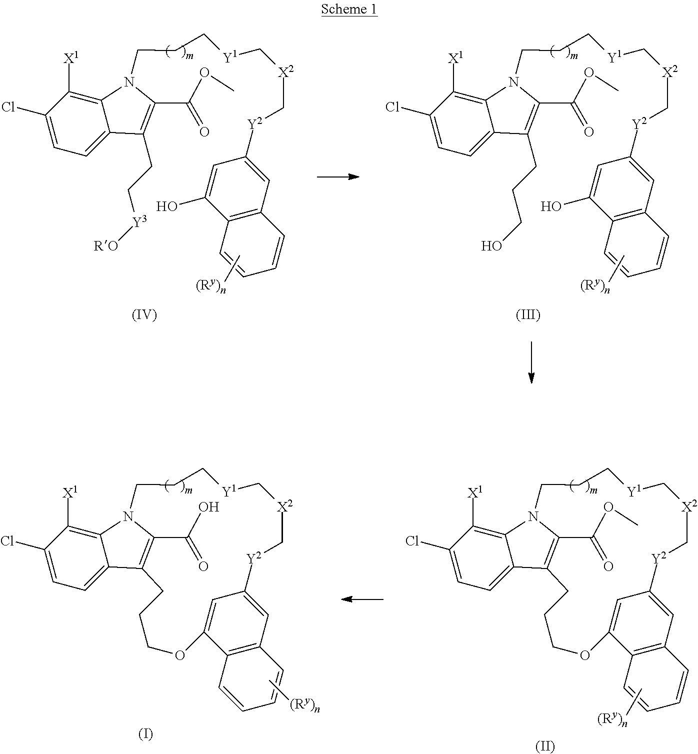

By reacting an intermediate of Formula (II) with a suitable base, such as, for example, LiOH or NaOH, in a suitable solvent, such as, for example, water or a mixture of water and a suitable organic solvent such as, for example, dioxane or THE (tetrahydrofuran), or a mixture of MeOH and THF, at a suitable temperature, such as, for example, room temperature or 60° C.
Intermediates of Formula (II) can be prepared by reacting an intermediate of Formula (III) with a suitable reagent such as, for example, diethyl azodicarboxylate (DEAD) or di-tert-butyl azodicarboxylate (DTBAD), in the presence of a suitable phosphine such as, for example, PPh₃, in a suitable solvent such as, for example, THF, toluene, or a mixture thereof, at a suitable temperature such as, for example, room temperature or 70° C.
Alternatively, Intermediates of Formula (II) can be prepared by reacting an intermediate of Formula (III) with a suitable reagent such as, for example, cyanomethylenetributylphosphorane (CAS [157141-27-0]), in a suitable solvent, such as, for example, ACN, or a mixture of ACN and THF, at a suitable temperature such as, for example, 80° C.
When Y³ is CH₂ and R′ is a silyl protecting group such as, for example, TBDMS or TBDPS in Intermediates of Formula (IV), Intermediates of Formula (III) can be prepared by reacting an intermediate of Formula (IV) with a suitable deprotecting agent such as, for example, HCl or p-toluenesulfonic acid (PTSA), in a suitable solvent such as, for example, methanol (MeOH), tetrahydrofuran (THF), or a mixture thereof, at a suitable temperature such as, for example, room temperature.
Alternatively, when Y³ is C═O and R′ is Me in Intermediates of Formula (IV), an additional reduction step might be necessary with a suitable reducing agent such as, for example, BH₃·DMS (borane dimethylsulfide), in a suitable solvent such as, for example, THF, at a suitable temperature such as, for example, room temperature or 50° C.

Alternatively, intermediates of Formula (II), when Y¹ is defined as S(O) or SO₂ and Y² is defined as CH₂, can also be prepared by reacting an intermediate of Formula (II), where Y¹ is defined as S (sulfur) and Y² is defined as CH₂, with a suitable oxidizing agent such as, for example, mCPBA (3-chloroperoxybenzoic acid), in a suitable solvent such as, for example, DCM (dichloromethane), at a suitable temperature such as, for example, room temperature.

An intermediate of Formula (II) might have a protecting group in the R¹ or R² position such as, for example, tetrahydropyranyl (THP). In such a case, the intermediate of Formula (II) is reacted with a suitable deprotection reagent, such as, for example, pTsOH (p-toluenesulfonic acid) or HCl, in a suitable solvent such as, for example, iPrOH (2-propanol), at a suitable temperature such as, for example, room temperature. In a next step the obtained unprotected intermediate can be reacted with a suitable alkylating agent R¹L (where L is as suitable leaving group) such as, for example, an alkyl halide, in the presence of a suitable base such as, for example, Cs₂CO₃, in a suitable solvent such as, for example, DMF (N,N-dimethylformamide), at a suitable temperature such as, for example, room temperature or 60° C.

It will be clear for someone skilled in the art, that orthogonality of protective groups will have to be considered in this case, for instance when R¹ is a tetrahydropyranyl, R′ should be preferably TBDMS or TBDPS groups, with Y³ being CH₂.

Intermediates of Formula (IV) wherein X¹, X², Y², R^{y}, m, and n are defined as in Formula (I), and Y³/R′ is C═O/Me or Y³/R′ is CH₂/TBDMS, can be prepared according to Scheme 2,

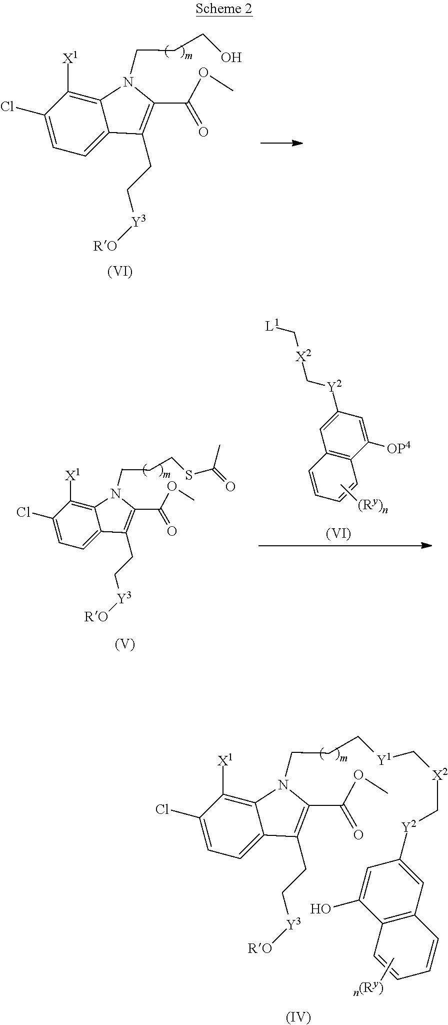

by reacting an intermediate of Formula (V), where X¹ and m are defined as in Formula (I) and Y³/R′ is C═O/Me or Y³/R′ is CH₂/TBDMS, with an intermediate of Formula (VI), where X², Y² and (R^{y})ₙ are defined as in Formula (I), P⁴ is a suitable protecting group such as, for example, TBDPS or TBDMS, and L¹ is defined as a suitable leaving group such as, for example, I, Br, Cl or OMs (methanesulfonate), in the presence of a suitable base such as, for example, K₂CO₃, in a suitable solvent such as, for example, MeOH, THF, or a mixture thereof, at a suitable temperature such as, for example, room temperature.
Intermediates of Formula (V) can be prepared by reacting an intermediate of Formula (VII), in a two-step procedure, first in the presence of a suitable activating agent such as, for example, methanesulfonyl chloride (MsCl), in the presence of a suitable base such as, for example, DIPEA, in a suitable solvent such as, for example, DCM, at a suitable temperature such as, for example, room temperature, then by reacting with potassium thioacetate (AcSK) in a suitable solvent such as, for example, ACN or DMF, at a suitable temperature such as, for example, room temperature or 60° C.

Intermediates of Formula (VII) wherein X¹ and m are defined as in Formula (I), and Y³/R′ is C═O/Me or Y³/R′ is CH₂/TBDMS, can be prepared according to Scheme 3,

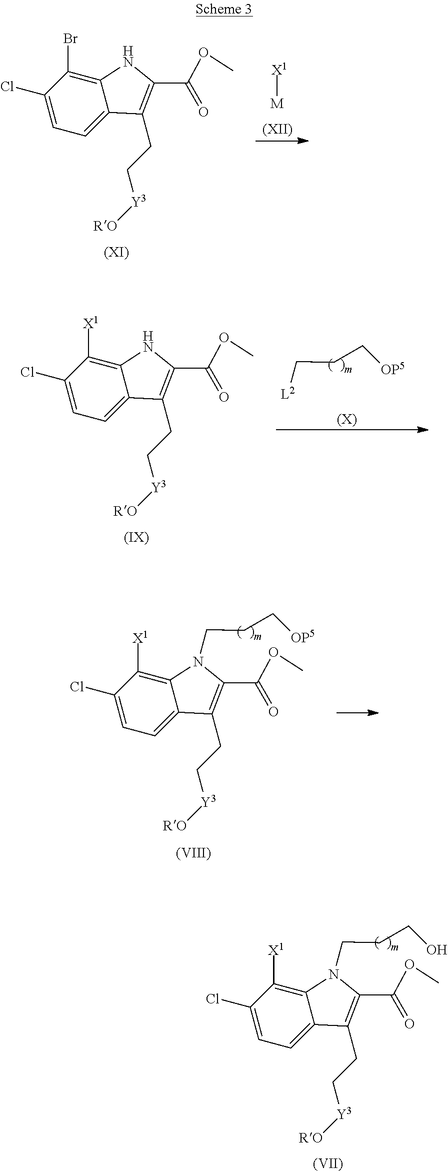

by reacting an intermediate of Formula (VIII) where P⁵ is a suitable protecting group such as, for example, PMB (p-methoxybenzyl) with suitable deprotecting agent such as, for example, DDQ (2,3-dichloro-5,6-dicyano-1,4-benzoquinone), in a suitable solvent such as, for example, a mixture of DCM and water, at a suitable temperature such as, for example, room temperature.
Intermediates of Formula (VIII) can be prepared by reacting an intermediate of Formula (IX) with an intermediate of Formula (X), where L² is a suitable leaving group such as, for example, MsO (mesylate) or I (iodide), in the presence of a suitable base such as, for example, Cs₂CO₃, in a suitable solvent such as, for example, DMF, at a suitable temperature such as, for example, 60° C.
Intermediates of Formula (IX) wherein Y³ is C═O, and R′ is Me, can be prepared by reacting an intermediate of Formula (XI) such as methyl 7-bromo-6-chloro-3-(3-methoxy-3-oxopropyl)-1H-indole-2-carboxylate (CAS [2143010-85-7]) with an intermediate of Formula (XII), in the presence of a suitable catalyst such as, for example, [1,1′-Bis(di-tert-butylphosphino)ferrocene]dichloropalladium(II) (Pd(dtbpf)Cl₂), in the presence of a suitable base such as, for example, Cs₂CO₃, in a suitable solvent such as, for example, a mixture of dioxane and water, at a suitable temperature such as, for example, 100° C.
Alternatively, this whole synthetic pathway may start from an intermediate of Formula (XI) such as methyl 7-bromo-6-chloro-3-(3-hydroxypropyl)-1H-indole-2-carboxylate (CAS [2245716-18-9]) after its protection with a suitable protecting group reagent such as, for example, TBDMSCl (tert-butyldimethylchlorosilane), in the presence of a suitable base such as, for example, Et₃N (triethylamine) or DMAP (4-dimethylaminopyridine), or a mixture thereof, in a suitable solvent such as, for example, THF, at a suitable temperature such as, for example, room temperature, leading to intermediates wherein Y³ is CH₂ and R′ is a suitable protecting group such as TBDMS.
Intermediates of Formula (X) are commercially available or can be prepared according to literature procedures.

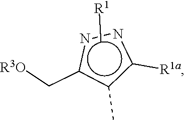

Intermediates of Formula (II-a), where X¹ is defined as, wherein R³, R¹ and R^{1a} are as defined in Formula (I), and X², Y², R^{y}, m, and n are defined as in Formula (I), can be prepared according to Scheme 4,

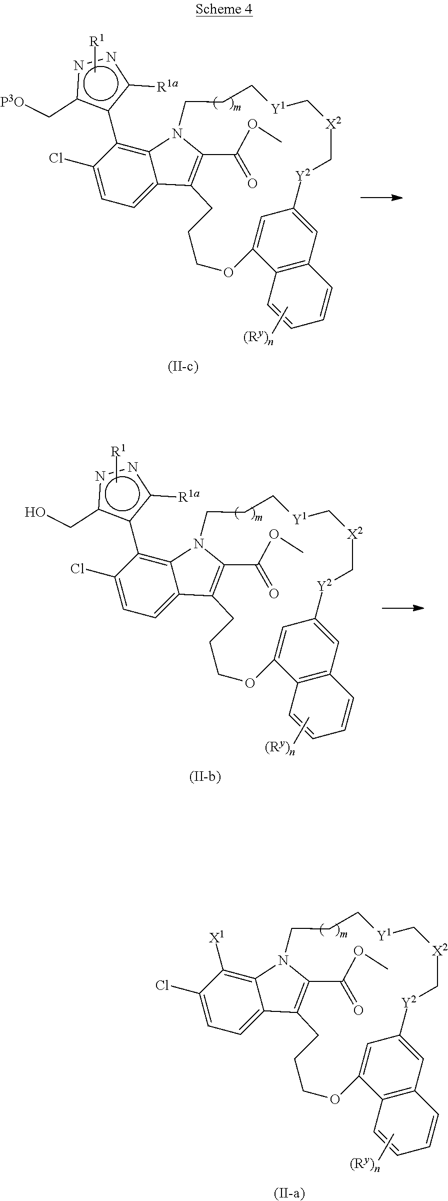

by reacting an intermediate of Formula (II-b) with a suitable alkylating agent such as, for example, 2-bromoethyl methyl ether, in the presence of a suitable base such as, for example, NaH, in a suitable solvent such as, for example, THF, at a suitable temperature such as, for example, 0° C. or room temperature.
Intermediates of Formula (II-b) can be prepared by reacting an intermediate of Formula (II-c), wherein P³ is a suitable protecting group such as, for example, THP, with a suitable deprotecting reagents such as, for example, PTSA, in a suitable solvent such as, for example, MeOH, at a suitable temperature such as, for example, room temperature.

Intermediates of Formula (VI), where R^{y}, R² and n are defined as in Formula (I), and and where P⁴ is a suitable protective group such as, for example, TBDPS, and where L¹ is a suitable leaving group such as, for example, iodide, can be prepared according to Scheme 5,

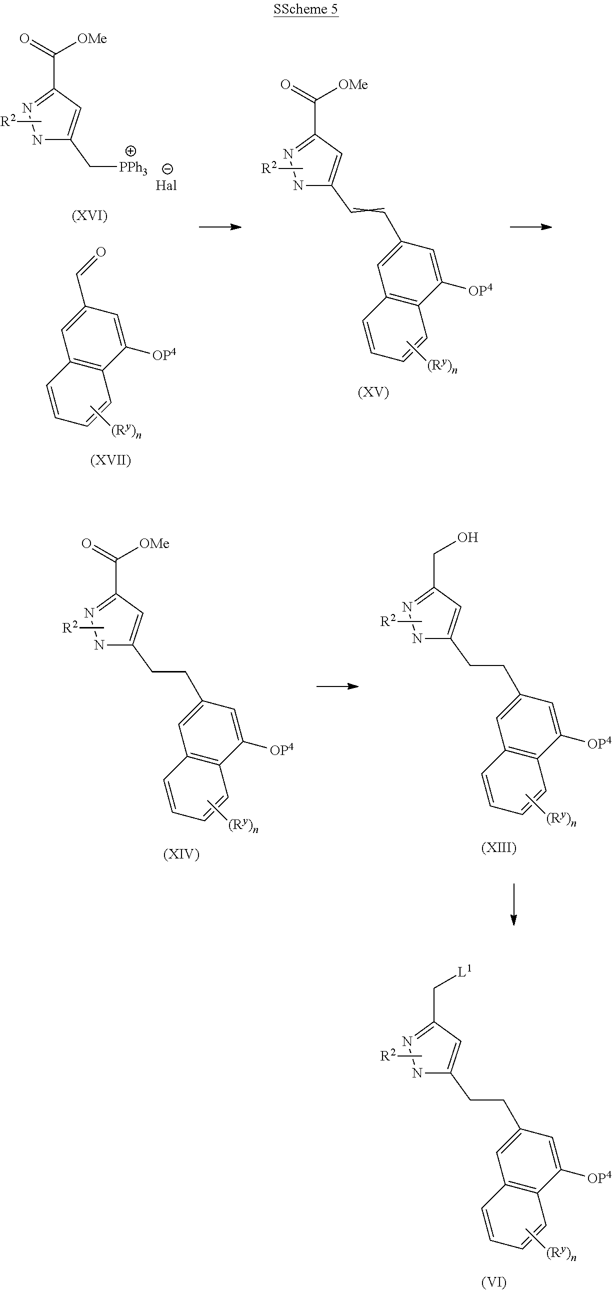

by reacting an intermediate of Formula (XIII) with a suitable activating agent such as, for example, MsCl, followed by a suitable leaving group precursor, such as, for example, NaI, in a suitable solvent such as, for example, THF, at a suitable temperature such as, for example, room temperature.
Intermediates of Formula (XIII) can be prepared by reacting an intermediate of Formula (XIV) with a suitable reducing agent such as, for example, LiAlH₄, in a suitable solvent, such as, for example, THF, at a suitable temperature, such as, for example, 0° C.
Intermediates of Formula (XIV) can be prepared by reacting an intermediate of Formula (XV), with a suitable hydrogenating reagent such as, for example, hydrogen gas, in the presence of a suitable catalyst such as, for example, Pd/C, in a suitable solvent such as, for example, MeOH, at a suitable temperature such as, for example, room temperature.
Intermediates of Formula (XV) can be prepared by reacting an intermediate of Formula (XVI) with an intermediate of Formula (XVII), in the presence of a suitable base such as, for example, NaH, in a suitable solvent such as, for example, THF, at a suitable temperature such as, for example, 0° C.
Intermediates of Formula (XVI) can be prepared in analogy with ((3-(methoxycarbonyl)-1-methyl-1H-pyrazol-5-yl)methyl)triphenylphosphonium chloride (CAS [2245716-31-6]).

A skilled person will understand that in Scheme 5, a protecting group might be present in the R² position such as, for example, tetrahydropyranyl (THP). Said protecting group can be removed as described hereinbefore.

Alternatively, intermediates of Formula (VI), where R^{y} R² and n are defined as in Formula (I), and where P⁴ is a suitable protective group such as, for example, TBDPS, and where L¹ is a suitable leaving group such as, for example, iodide, can be prepared according to Scheme 6,

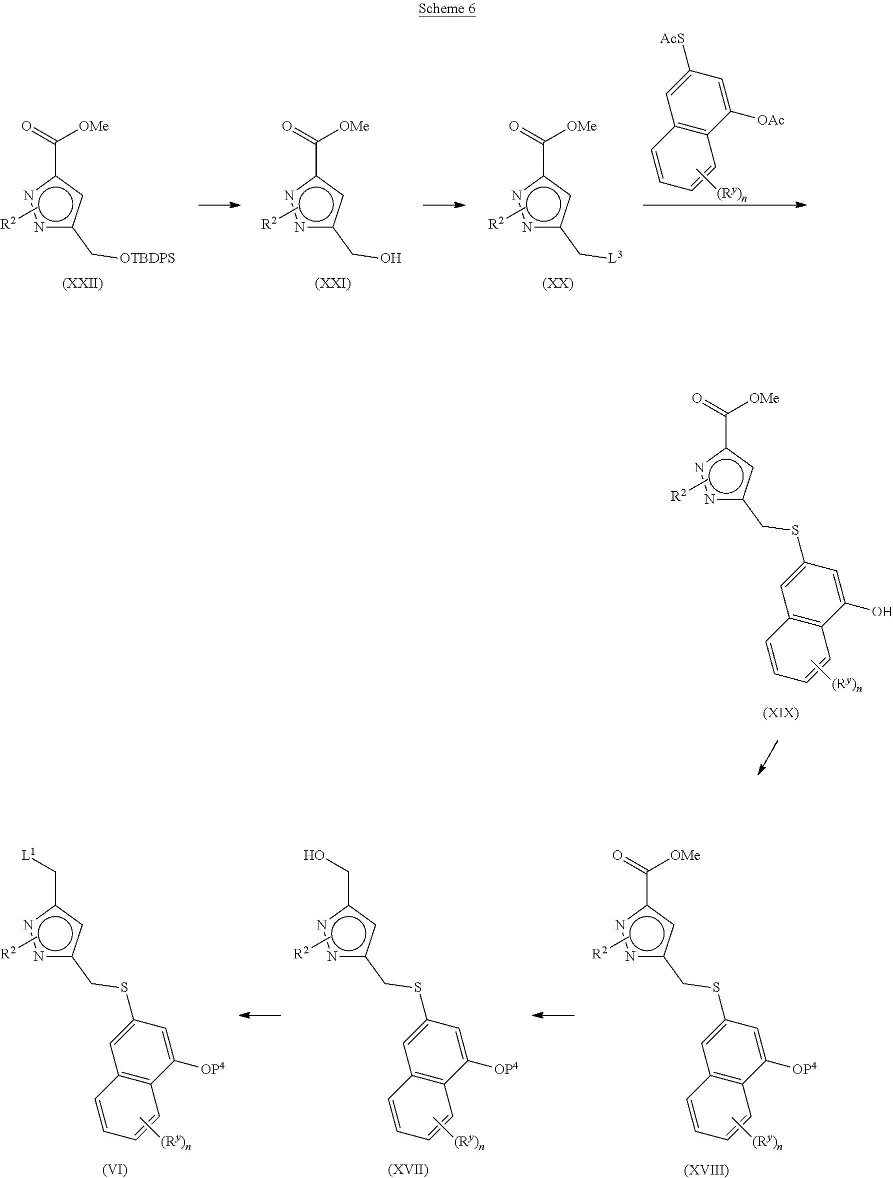

by reacting an intermediate of Formula (XVII) with a suitable activating agent such as, for example, MsCl, followed by a suitable leaving group precursor, such as, for example, NaI, in a suitable solvent such as, for example, THF, at a suitable temperature such as, for example, room temperature.
Intermediates of Formula (XVII) can be prepared by reacting an intermediate of Formula (XVIII), with a suitable reducing agent such as, for example, DIBALH, in a suitable solvent, such as, for example, THF, at a suitable temperature, such as, for example, 0° C. or room temperature.
Intermediates of Formula (XVIII) can be prepared by reacting an intermediate of Formula (XIX), with a suitable trisubstituted silyl chloride such as, for example, TBDMSCl (tert-butyldimethylsilyl chloride) or TBDPSCl (tert-butyldiphenylsilyl chloride), in the presence of a suitable base, such as, for example, imidazole, in a suitable solvent, such as, for example, DMF, at a suitable temperature, such as, for example, room temperature.
Intermediates of Formula (XIX) can be prepared by reacting an intermediate of Formula (XX) where L³ is a suitable leaving group, such as, for example, chloride or mesylate, with a suitably substituted 3-(acetylthio)naphthalen-1-yl acetate, in the presence of a suitable base, such as, for example, K₂CO₃, in a suitable solvent, such as, for example, methanol, at a suitable temperature, such as, for example, room temperature.
Intermediates of Formula (XX) can be prepared by reacting an intermediate of Formula (XXI), with a suitable reagent such as, for example, mesyl chloride or thionyl chloride, if necessary in the presence of a suitable base, such as, for example, triethylamine, in a suitable solvent, such as, for example, CH₂Cl₂, at a suitable temperature, such as, for example, 0° C. or room temperature.
Intermediates of Formula (XXI) can be prepared by reacting an intermediate of Formula (XXII), with a deprotecting agent, such as, for example, TBAF, in a suitable solvent, such as, for example THF, at a suitable temperature, such as, for example, room temperature.
Intermediates of Formula (XXII) can be prepared by reacting ethyl 5-(((tert-butyldiphenylsilyl)oxy)methyl)-1H-pyrazole-3-carboxylate, with a suitable protecting group precursor, such as, for example, paramethoxybenzyl chloride, dimethoxylbenzyl chloride, or also a suitable alkyl halide, such as, for example, methyl iodide (will afford the methylated pyrazole instead of the protected pyrazole), in the presence of suitable base, such as, for example, sodium bis(trimethylsilyl)amide, in a suitable solvent, such as, for example THF, at a suitable temperature, such as, for example, 0° C. or room temperature.
Alternatively, intermediates of Formula (XXII) can be prepared by reacting ethyl 5-(((tert-butyldiphenylsilyl)oxy)methyl)-1H-pyrazole-3-carboxylate, with a suitable protecting group precursor, such as, for example, 3,4-dihydro-2H-pyran, in the presence of suitable catalyst, such as, for example, p-toluenesulfonic acid (PTSA), in a suitable solvent, such as, for example tetrahydrofuran (THF) or CH₂Cl₂, at a suitable temperature, such as, for example, 0° C. or room temperature.

A skilled person will understand that in Scheme 6, a protecting group might be present in the R² position such as, for example, tetrahydropyranyl (THP). Said protecting group can be removed as described hereinbefore.

Intermediates of Formula (XVII), where R^{y} and n are defined as in Formula (I), wherein P⁴ is a suitable protective group such as, for example, TBDPS, can be prepared according to Scheme 7,

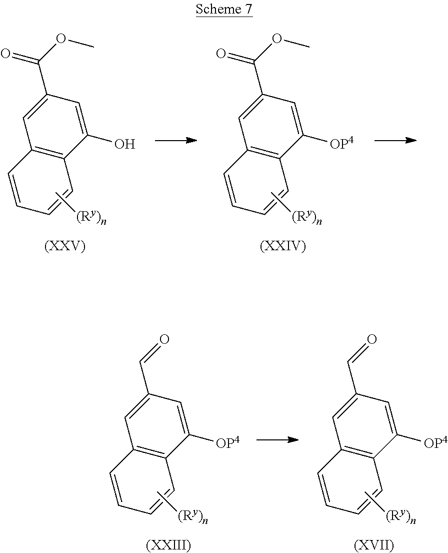

By reacting an intermediate of Formula (XXIII) with a suitable oxidizing agent such as, for example, MnO₂, in a suitable solvent such as, for example, CH₃CN, at a suitable temperature such as, for example, 60° C.
Intermediates of Formula (XXIII) can be prepared by reacting an intermediate of Formula (XXIV) with a suitable reducing agent such as, for example, LiAlH₄, in a suitable solvent such as, for example, THF, at a suitable temperature such as, for example, 0° C.
Intermediates of Formula (XXIV) can be prepared by reacting an intermediate of Formula (XXV) with a suitable protecting group such as, for example, TBDPSCl, in the presence of a suitable base such as, for example, imidazole, in a suitable solvent such as, for example, DMF, at a suitable temperature such as, for example, room temperature.
Intermediates of Formula (XXV) are commercially available or can be prepared similarly to methyl 7-fluoro-4-hydroxy-2-naphthoate (CAS [2092726-85-5]).

Intermediates of Formula (XII) are commercially available or can be prepared according to literature procedures.

Alternatively, intermediates of Formula (XII) wherein R¹, R^{1a} are defined as in Formula (I) and, and wherein P³ is a suitable protecting group such as, for example, TBDMS or THP; and B(OR)₂ is defined as a boronic acid or suitable boronate ester such as, for example, a pinacol ester, can be prepared according to Scheme 8,

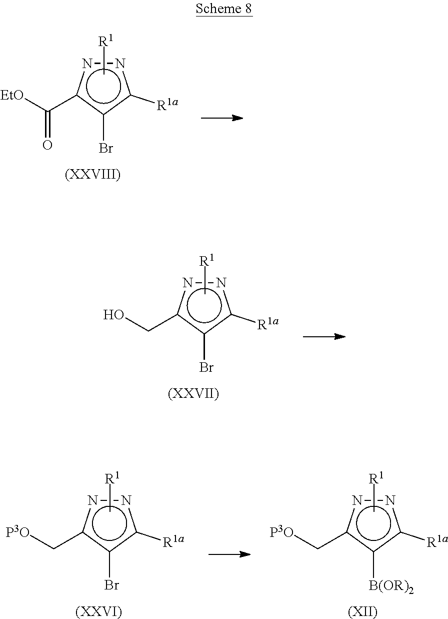

By reacting an intermediate of Formula (XXVI) with a suitable boronate such as, for example, isopropoxyboronic acid pinacol ester, in the presence of a suitable base such as, for example, BuLi, in a suitable solvent, such as, for example, THF, at a suitable temperature such as, for example, −78° C.
Intermediates of Formula (XXVI) can be prepared by reacting an intermediate of Formula (XXVII) with a suitable protecting group precursor such as, for example, TBDMSCl, in the presence of a suitable base such as, for example, Et₃N or DMAP, or a mixture thereof, in a suitable solvent such as, for example, THF, at a suitable temperature such as, for example, room temperature.
Intermediates of Formula (XXVII) can be prepared by reacting an intermediate of Formula (XXVIII) with a suitable reducing agent such as, for example, LiBH₄, in a suitable solvent such as, for example, 2-methyltetrahydrofuran (2-MeTHF), at a suitable temperature such as, for example, room temperature.
Intermediates of Formula (XXVIII) can be prepared can be prepared by standard synthetic processes commonly used by those skilled in the art of organic chemistry.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, New Jersey, 2007.

### Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NIL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8. Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### EXAMPLES

Several methods for preparing the Compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| ACN | acetonitrile |
| AcOH | acetic acid |
| Celite ® | diatomaceous earth |
| Co | Compound |
| Co. No. | Compound Number |
| DCM | dichloromethane |
| DIBALH | di-isobutylaluminiumhydride |
| DDQ | 2,3-Dichloro-5,6-dicyano-1,4-benzoquinone |
| DEA | diethylamine |
| DIPEA | N,N-diisopropylethylamine |
| DMAP | 4-dimethylaminopyridine |
| DMF | N,N-dimethylformamide |
| eq. | equivalent(s) |
| Et₃N or TEA | triethylamine |
| EtOAc or AcOEt | ethyl acetate |
| EtOH | ethanol |
| HPLC | high performance liquid chromatography |
| iPrNH₂ | isopropylamine |
| iPrOH | isopropanol |
| mCPBA | meta-chloroperoxybenzoic acid |
| Me | methyl |
| MeOH | methanol |
| Me-THF or | 2-methyltetrahydrofuran |
| 2-Me-THF | |
| MsCl | methanesulfonyl chloride |
| NaBH(OAc)₃ | sodium triacetoxyborohydride |
| PPh₃ | triphenylphosphine |
| quant. | quantitative |
| rac | racemic |
| RP | reversed phase |
| SFC | supercritical fluid chromatography |
| TBAF | tetrabutylammonium fluoride |
| TBDMSCl | tert-butyldimethylsilyl chloride |
| TBDPSCl | tert-butyldiphenylsilyl chloride |
| THF | tetrahydrofuran |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

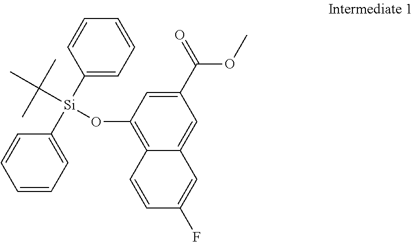 [SMILES: COC(=O)c1cc(O[Si](c2ccccc2)(c2ccccc2)C(C)(C)C)c2ccc(F)cc2c1]

TBDPSCl (14.66 g, 1.5 eq.) was added to a solution of methyl 7-fluoro-4-hydroxy-2-naphthoate (CAS [2092726-85-5], 8 g, 35.555 mmol) and imidazole (7.26 g, 3 eq.) in DCM (500 mL), cooled to 0° C. under nitrogen atmosphere. Once the addition was complete, the reaction mixture was stirred at room temperature overnight. The reaction was quenched by addition of water (100 mL). The mixture was extracted with EtOAc (3×200 mL). The combined organic layer was dried over Na₂SO₄, filtered, and concentrated to afford a yellow oil. This oil was purified by flash column chromatography on silica gel (petroleum ether:EtOAc—1:0 to 1:1) to afford Intermediate 1 (14 g, yield: 86%) as a yellow oil.

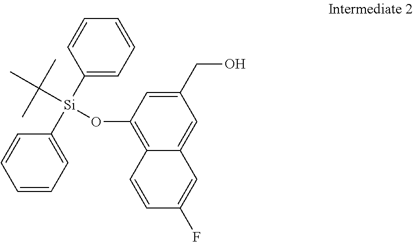 [SMILES: CC(C)(C)[Si](Oc1cc(CO)cc2cc(F)ccc12)(c1ccccc1)c1ccccc1]

LiAlH₄ (1.39 g, 1.2 eq.) was added slowly to a solution of Intermediate 1 (14 g, 30.528 mmol) in THE (200 mL), cooled to 0° C. under nitrogen atmosphere. Once the addition was complete, the reaction mixture was stirred at 0° C. for 2 h. The reaction was quenched by slow addition of water (2 mL) followed by a 10% aqueous NaOH solution (2 mL) at 0° C. The heterogeneous mixture was filtered, and the filter cake was washed with DCM (200 mL). The filtrate was evaporated and the residue was purified by flash column chromatography on silica gel (petroleum ether:EtOAc—1:0 to 1:1) to give Intermediate 2 (12 g, yield: 90%) as a yellow solid.

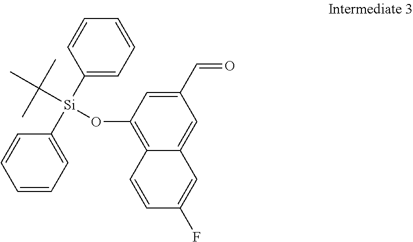 [SMILES: CC(C)(C)[Si](Oc1cc(C=O)cc2cc(F)ccc12)(c1ccccc1)c1ccccc1]

MnO₂ (29.074 g, 12 eq.) was added to a solution of Intermediate 2 (12 g, 27.869 mmol) in DCM (200 mL) at room temperature. The resulting solution was stirred at room temperature overnight. The reaction mixture was filtered and the filtrate was concentrated. The residue was purified by flash column chromatography over silica gel (eluent: petroleum ether/EtOAc, 100/0 to 50/50) to afford Intermediate 3 (12 g, yield: 99%) as a yellow oil.

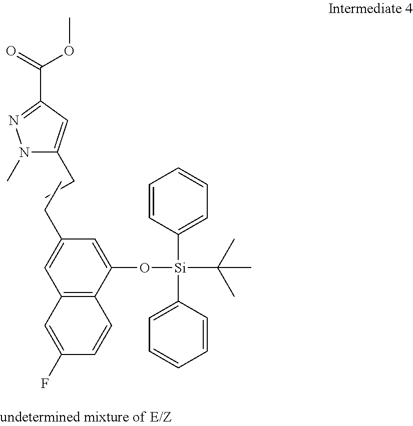

NaH (60% in mineral oil, 1.448 g, 1.3 eq.) was added to a suspension of ((3-(methoxycarbonyl)-1-methyl-1H-pyrazol-5-yl)methyl)triphenylphosphonium chloride (CAS [2245716-31-6], 13.812 g, 1.1 eq.) in THF (200 mL) at 0° C. The resulting solution was stirred at this temperature for 1 h before being cooled to −30° C. Intermediate 3 (12 g, 27.847 mmol) was added slowly to the solution while maintaining the temperature between −20° C. and −30° C. Once the addition was complete, the reaction was stirred at −30° C. for 2 h. The reaction was quenched by slow addition of water (100 mL). The mixture was extracted with DCM (3×300 mL). The combined organic layer was dried over Na₂SO₄, filtered, and concentrated under reduced pressure. The crude product was purified by column chromatography on silica gel (petroleum ether/EtOAc,—gradient from 1:0 to 1:1) to afford Intermediate 4 (13 g, yield: 82%) as a white solid.

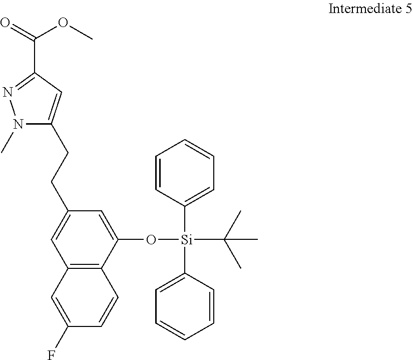 [SMILES: COC(=O)c1cc(CCc2cc(O[Si](c3ccccc3)(c3ccccc3)C(C)(C)C)c3ccc(F)cc3c2)n(C)n1]

A solution of Intermediate 4 (13 g, 23.02 mmol) in MeOH (75 mL) and THF (75 mL) was hydrogenated at 25° C. (15 psi H₂) in the presence of Pd/C (2 g). The reaction mixture was stirred for 16 h. After uptake of H₂ (1 eq.), the catalyst was filtered off and the filtrate was evaporated to afford Intermediate 5 (13 g, yield: 100%) as a colorless oil.

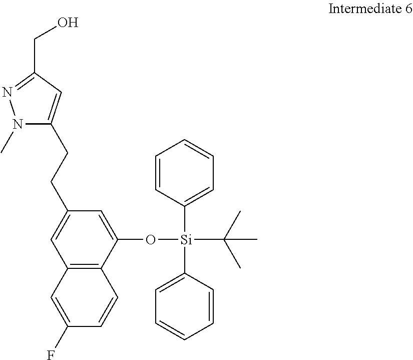 [SMILES: Cn1nc(CO)cc1CCc1cc(O[Si](c2ccccc2)(c2ccccc2)C(C)(C)C)c2ccc(F)cc2c1]

LiAlH₄ (1.045 g, 1.2 eq.) was added portionwise to a solution of Intermediate 5 (13 g, 22.94 mmol) in THF (200 mL) at 0° C., under nitrogen atmosphere. The reaction mixture was stirred at 0° C. for 2 h. Water (1 mL) was then added dropwise, followed by a 10% aqueous NaOH solution (1 mL), at 0° C. The reaction mixture was filtered, the filter cake was washed with DCM (200 mL), and the filtrate was evaporated. The crude product was purified by flash column chromatography over silica gel (eluent: petroleum ether/EtOAc, 100/0 to 0/100) to afford Intermediate 6 (10.4 g, yield: 84%) as a white solid.

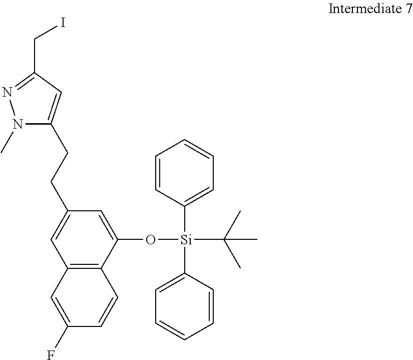 [SMILES: Cn1nc(CI)cc1CCc1cc(O[Si](c2ccccc2)(c2ccccc2)C(C)(C)C)c2ccc(F)cc2c1]

DIPEA (0.64 mL, 2 eq.) followed by methanesulfonic anhydride (0.65 g, 2 eq.) was added to a solution of Intermediate 6 (1.0 g, 1.86 mmol) in THF (45 mL), cooled to 0° C. The reaction mixture was stirred at room temperature for 0.5 h. Sodium iodide (1.39 g, 5 eq.) was then added to the mixture and it was further stirred at room temperature for 1 h. The reaction mixture was diluted with DCM (100 mL) and washed with water (20 mL). The aqueous layer was extracted with DCM/iPrOH 3:1 (2×30 mL), the combined organic layer was dried over MgSO₄, filtered, and concentrated under reduced pressure to give a dark yellow oil. This oil was purified by flash column chromatography on silica gel (SiO₂, 24 g column, 0-3% MeOH in DCM) to give Intermediate 7 (1.1 g, yield: 91%).

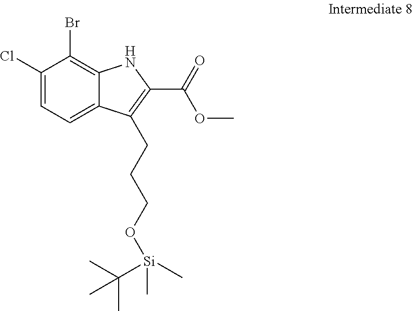 [SMILES: COC(=O)c1[nH]c2c(Br)c(Cl)ccc2c1CCCO[Si](C)(C)C(C)(C)C]

Tert-butyldimethylsilyl chloride (2.06 g, 1.4 eq.) was added portionwise to a mixture of methyl 7-bromo-6-chloro-3-(3-hydroxypropyl)-1H-indole-2-carboxylate (CAS [2245716-18-9], 3.5 g, 9.78 mmol) and imidazole (1 g, 1.5 eq.) in DCM (80 mL) at 0° C. DMAP (59 mg, 0.05 eq.) was then added and the reaction mixture was stirred at room temperature for 1 h. The reaction mixture was diluted with DCM and washed with water. The organic layer was separated, dried on MgSO₄, filtered, and evaporated to give Intermediate 8 (4.46 g, 87% yield), used without further purification.

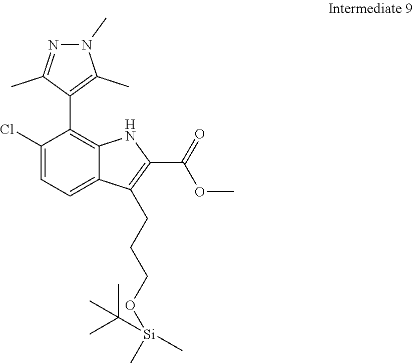

A solution of Intermediate 8 (2.378 g, 5.159 mmol), 1,3,5-trimethyl-4-(4,4,5,5-tetramethyl-1,3,2-dioxaborolan-2-yl)-1H-pyrazole (CAS [844891-04-9], 1.34 g, 5.675 mmol, 1.1 eq.), and Cs₂CO₃ (2.521 g, 7.739 mmol, 1.5 eq.) in water (15 mL) and 1,4-dioxane (35 mL) was prepared and degassed with nitrogen for 10 min. 1,1′-Bis(ditert-butylphosphino)ferrocene-palladium dichloride (CAS [95408-45-0], 340 mg, 0.516 mmol, 0.1 eq.) was then added to the solution and it was further degassed for 5 min. The red solution was then distributed in 3 microwave vials. Each vial was sealed and stirred at 100° C. for 2 h in a microwave oven. After cooling, the vials were pooled, and the reaction mixture was diluted with water and EtOAc. The layers were separated and the aqueous layer was extracted again with EtOAc. The combined organic layer was washed with brine, dried on MgSO₄, filtered, and evaporated. The residue was purified by column chromatography (Biotage Sfar 100 g; eluent: AcOEt/EtOH 3/1:n-heptane, gradient from 0:100 to 40:60) to give Intermediate 9 (2.476 g, yield: 98%) as a thick brown oil, crystallizing upon standing, and dried under vacuum at 50° C.

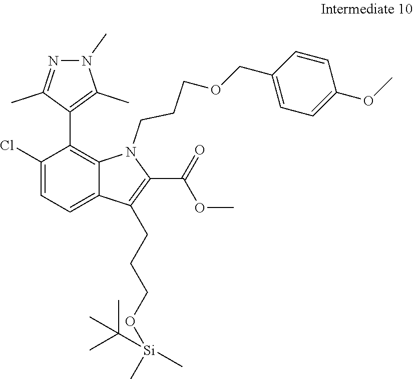

Cesium carbonate (1953 mg, 5.99 mmol, 1.5 eq.) was added to a solution of Intermediate 9 (1997 mg, 3.99 mmol) and 1-[(3-iodopropoxy)methyl]-4-methoxybenzene (CAS [198411-17-5], 1834 mg, 5.99 mmol, 1.5 eq.) in dry DMF (50 mL) at room temperature. The reaction mixture was stirred at 60° C. for 3 h. The mixture was diluted with EtOAc and water. The layers were separated and the aqueous layer was extracted again with EtOAc. The combined organic layer was washed with brine, dried on MgSO₄, filtered, and the solvent was evaporated. The residue was purified by column chromatography (Biotage Sfar 100 g; eluent: AcOEt/EtOH 3/1:n-heptane, gradient from 0:100 to 40:60) to give Intermediate 10 (1940 mg, yield: 73%) as a thick black oil, dried under vacuum at 50° C.

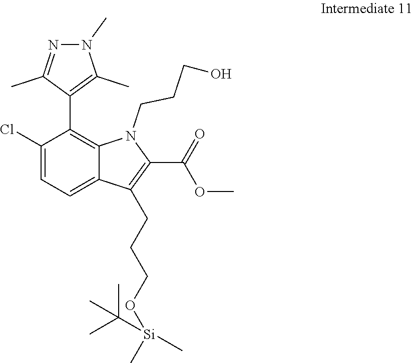

2,3-dichloro-5,6-dicyano-1,4-benzoquinone (1318 mg, 5.80 mmol, 2 eq.) was added to a solution of Intermediate 10 (1940 mg, 2.90 mmol) in DCM (40 mL) and water (5 mL). The reaction mixture was stirred at room temperature for 1 h. The reaction mixture was diluted with DCM and saturated aqueous NaHCO₃. The layers were separated and the aqueous layer was extracted again with DCM. The combined organic layer was dried on MgSO₄, filtered, and evaporated. The residue was purified by column chromatography (Biotage Sfar 50 g; eluent: AcOEt/EtOH 3/1:n-heptane, gradient from 0:100 to 40:60) to give Intermediate 11 as a brown solid (1007 mg, yield: 63%), dried under vacuum at 50° C.

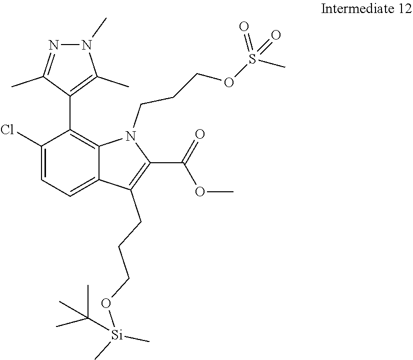

Methanesulfonyl chloride (170 μL, 2.189 mmol, 1.2 eq.) was added dropwise to a solution of Intermediate 11 (1000 mg, 1.824 mmol) and DIPEA (377 μL, 2.189 mmol, 1.2 eq.) in dry DCM (40 mL) at room temperature. The reaction mixture was stirred at room temperature for 2 h. To push the reaction to completion, additional methanesulfonyl chloride (42 μL, 0.547 mmol, 0.3 eq.) and DIPEA (94 μL, 0.547 mmol, 0.3 eq.) were added dropwise to the reaction mixture and it was further stirred at room temperature for 2.5 h. Water was added and the layers were separated. The aqueous layer was extracted again with CH₂Cl₂. The combined organic layer was dried on MgSO₄, filtered, and evaporated. The residue was purified by column chromatography (Biotage Sfar 50 g; eluent: AcOEt/EtOH 3/1:n-heptane, gradient from 0:100 to 40:60) to give Intermediate 12 (974 mg, yield: 85%) as a thick brown oil, dried under vacuum at 50° C.

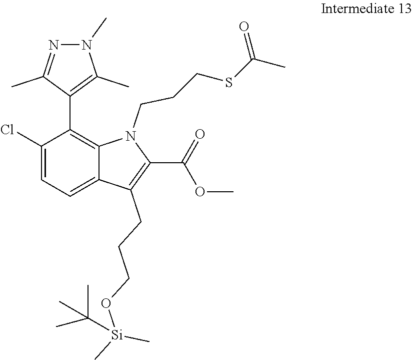 [SMILES: COC(=O)c1c(CCCO[Si](C)(C)C(C)(C)C)c2ccc(Cl)c(-c3c(C)nn(C)c3C)c2n1CCCSC(C)=O]

Potassium thioacetate (533 mg, 4.666 mmol, 3 eq.) was added to a solution of Intermediate 12 (974 mg, 1.555 mmol) in dry ACN (30 mL). The reaction mixture was then stirred at 60° C. for 5 h. The solvent was evaporated and the residue was partitioned between water and EtOAc. The layers were separated and the aqueous layer was extracted again with EtOAc. The combined organic layer was dried on MgSO₄, filtered, and evaporated. The residue was purified by column chromatography (Biotage Sfar 50 g; eluent: AcOEt/EtOH 3/1:n-heptane, gradient from 0:100 to 40:60) to give Intermediate 13 (800 mg, yield: 85%) as a thick yellow oil, dried under vacuum at 50° C.

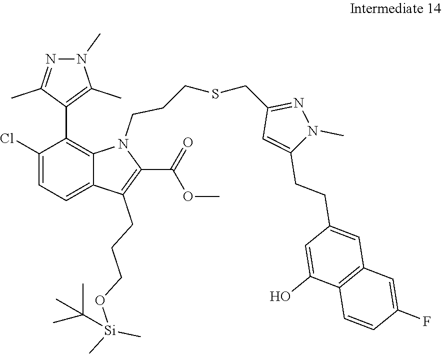 [SMILES: COC(=O)c1c(CCCO[Si](C)(C)C(C)(C)C)c2ccc(Cl)c(-c3c(C)nn(C)c3C)c2n1CCCSCc1cc(CCc2cc(O)c3ccc(F)cc3c2)n(C)n1]

Intermediate 13 (810 mg, 1.336 mmol) was dissolved in dry MeOH (30 mL) and this solution was degassed with nitrogen for 5 min (solution A). A suspension of Intermediate 7 (1025 mg, 1.47 mmol, 1.1 eq.) and K₂CO₃ (369 mg, 2.672 mmol, 2 eq.) in dry MeOH (30 mL) and THF (30 mL) was degassed with nitrogen for 5 min (solution B). Solution A was then added dropwise to solution B at room temperature under nitrogen atmosphere. The reaction mixture was stirred at room temperature under nitrogen atmosphere for 2 h. The solvents were evaporated and the residue was partitioned between water and EtOAc. The layers were separated and the aqueous layer was extracted again with EtOAc. The combined organic layer was dried on MgSO₄, filtered, and evaporated to give Intermediate 14 (1131 mg, assumed quantitative) as a thick brown oil, used without further purification.

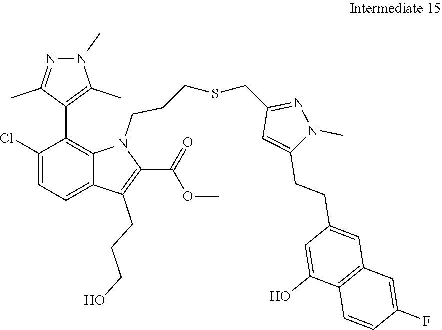 [SMILES: COC(=O)c1c(CCCO)c2ccc(Cl)c(-c3c(C)nn(C)c3C)c2n1CCCSCc1cc(CCc2cc(O)c3ccc(F)cc3c2)n(C)n1]

PTSA monohydrate (1289 mg, 6.674 mmol, 5 eq.) was added to a solution of Intermediate 14 (1130 mg, 1.335 mmol) in dry MeOH (100 mL) at room temperature. The reaction mixture was stirred at room temperature for 1 h. The solvent was evaporated and the residue was taken up in EtOAc and a saturated aqueous NaHCO₃ solution. The layers were separated and the aqueous layer was extracted again with EtOAc. The combined organic layer was dried on MgSO₄, filtered, and evaporated. The residue was purified by column chromatography (Biotage Sfar 50 g; eluent: AcOEt/EtOH 3/1:n-heptane, gradient from 0:100 to 100:0) to give Intermediate 15 (630 mg, 80% pure, yield: 52%) as a light brown solid.

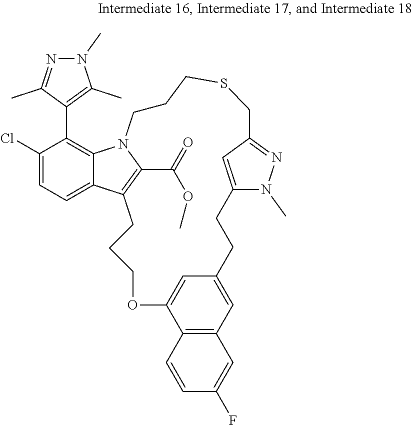

### Intermediate 16: Mixture of Atropisomers

### Intermediate 17: Rₐ or Sₐ; One Atropisomer but Absolute Stereochemistry Undetermined

### Intermediate 18: Sₐ or Rₐ; One Atropisomer but Absolute Stereochemistry Undetermined

Cyanomethylenetributylphosphorane (CAS [157141-27-0], 590 μL, 2.632 mmol, 4 eq.) was added to a solution of Intermediate 15 (595 mg, 0.658 mmol) in dry THF (2 mL) and dry ACN (50 mL) at room temperature under nitrogen atmosphere. The reaction mixture was stirred at 80° C. for 2.5 h under nitrogen atmosphere. The solvent was evaporated and the residue was purified by column chromatography (Biotage Sfar 25 g; eluent: AcOEt/EtOH 3/1:n-heptane, gradient from 0:100 to 100:0) to give Intermediate 16 (125 mg, yield: 27%) as a light brown solid. Intermediate 16 was separated into its atropisomers by preparative SFC (Stationary phase: Chiralpak Diacel AD 20×250 mm, Mobile phase: CO₂, EtOH-iPrOH (50-50)+0.4% iPrNH₂) to afford Intermediate 17 (49 mg, yield: 10%) and Intermediate 18 (44 mg, yield: 9%), both as white solids.

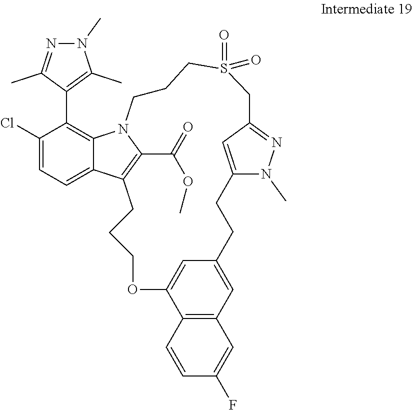

### Intermediate 19: Rₐ or Sₐ; One Atropisomer but Absolute Stereochemistry Undetermined

mCPBA (14 mg, 0.083 mmol, 2.2 eq.) was added in one portion to a solution of Intermediate 17 (27 mg, 0.038 mmol) in DCM (3 mL) at room temperature. The reaction mixture was stirred overnight at room temperature. The reaction mixture was diluted with DCM and saturated aqueous NaHCO₃. After vigorous stirring, the layers were separated and the aqueous layer was extracted again with DCM. The combined organic layer was dried by filtration on Extrelut NT3 (Supelco®), and evaporated. The residue was purified by column chromatography (Biotage Sfar 10 g; eluent: AcOEt/EtOH 3/1:n-heptane, gradient from 0:100 to 100:0) to give Intermediate 19 (24 mg, yield: 85%) as a white solid.

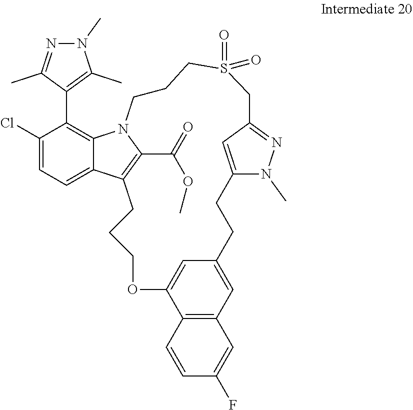

### Intermediate 20: Sₐ or Rₐ; One Atropisomer but Absolute Stereochemistry Undetermined

Intermediate 20 was prepared according to an analogous procedure as for Intermediate 19, starting from Intermediate 18 instead of Intermediate 17.

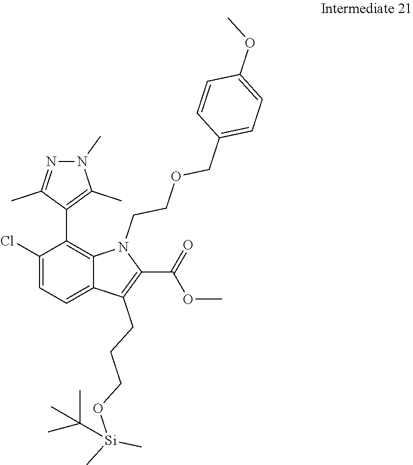 [SMILES: COC(=O)c1c(CCCO[Si](C)(C)C(C)(C)C)c2ccc(Cl)c(-c3c(C)nn(C)c3C)c2n1CCOCc1ccc(OC)cc1]

Cs₂CO₃ (5.285 g, 16.22 mmol, 3 eq.) was added to a solution of Intermediate 9 (2.65 g, 5.407 mmol) and ethanol, 2-[(4-methoxyphenyl)methoxy]-, 1-methanesulfonate (CAS [447454-24-2], 2.815 g, 10.81 mmol) in anhydrous DMF (20 mL). The reaction mixture was stirred at 60° C. for 16 h. The mixture was diluted with EtOAc (150 mL) and water (100 mL). The layers were separated and the organic one was washed with brine (2×50 mL). The combined aqueous layer was back-extracted with EtOAc (50 mL). The combined organic layer was dried over MgSO₄, filtered, and evaporated. The residue was purified by flash column chromatography on silica gel (120 g, gradient: from heptane/EtOAc 100/0 to 40/60) to afford Intermediate 21 (2.81 g, yield: 79%) as a yellow paste.

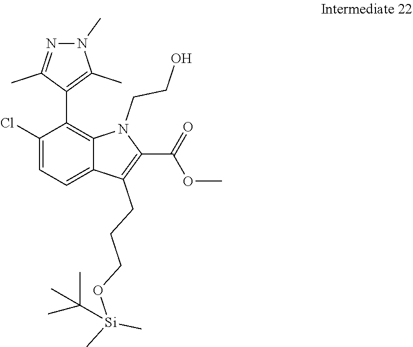

2,3-Dichloro-5,6-dicyano-1,4-benzoquinone (1.95 g, 8.589 mmol, 2 eq.) was added to a solution of Intermediate 21 (2.81 g, 4.295 mmol) in DCM (100 mL) and water (10 mL). The reaction mixture was stirred at room temperature for 1 h. The reaction mixture was diluted with DCM (250 mL) and water (150 mL). The layers were separated and the aqueous one was extracted with DCM (100 mL). The combined organic layer was washed with brine (100 mL), dried over MgSO₄, filtered, and concentrated under reduced pressure to give Intermediate 22 (2.294 g, assumed quantitative) as a brown paste, used without further purification in the next step.

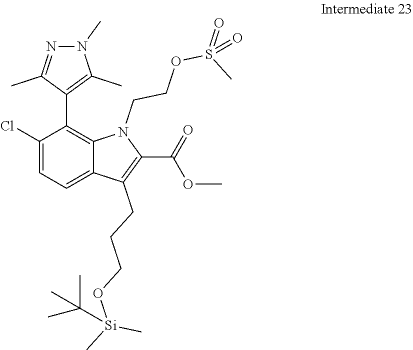 [SMILES: COC(=O)c1c(CCCO[Si](C)(C)C(C)(C)C)c2ccc(Cl)c(-c3c(C)nn(C)c3C)c2n1CCOS(C)(=O)=O]

Intermediate 22 (crude from previous step, 2.294 g, 4.295 mmol) was dissolved in dry DCM (100 mL) and the solution was cooled down to 0° C. Et₃N (3.58 mL, 25.76 mmol, 6 eq.) and MsCl (1.67 mL, 21.47 mmol, 5 eq.) were added and the reaction mixture was then stirred at room temperature for 30 min. The reaction mixture was diluted with DCM (150 mL) and water (100 mL). The organic layer was separated, dried over MgSO₄, filtered, and evaporated. The residue was purified by flash column chromatography on silica gel (120 g, gradient: from heptane/EtOAc 100/0 to 0/100) to afford Intermediate 23 (1.885 g, yield: 72% over 2 steps) as a yellowish paste.

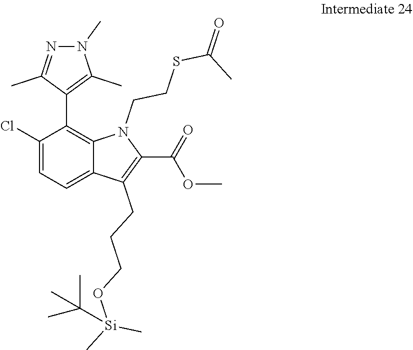 [SMILES: COC(=O)c1c(CCCO[Si](C)(C)C(C)(C)C)c2ccc(Cl)c(-c3c(C)nn(C)c3C)c2n1CCSC(C)=O]

Potassium thioacetate (70 mg, 0.612 mmol, 3 eq.) was added to a solution of Intermediate 23 (125 mg, 0.204 mmol) in anhydrous DMF (2 mL). The reaction mixture was stirred at 60° C. for 2 h. The reaction mixture was cooled down to room temperature and was diluted with EtOAc (30 mL) and water (20 mL). The organic layer was separated and washed with brine (2×15 mL). The combined aqueous layer was back-extracted with EtOAc (20 mL). The combined organic layer was dried over MgSO₄, filtered, and evaporated. The residue was purified by flash column chromatography on silica gel (12 g, gradient: heptane/EtOAc 100/0 to 50/50) to afford Intermediate 24 (95 mg, yield: 79%) as a colorless oil.

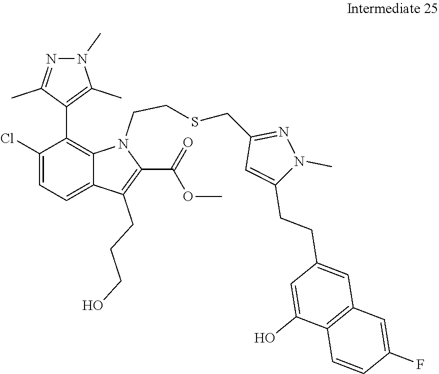 [SMILES: COC(=O)c1c(CCCO)c2ccc(Cl)c(-c3c(C)nn(C)c3C)c2n1CCSCc1cc(CCc2cc(O)c3ccc(F)cc3c2)n(C)n1]

Intermediate 24 (95 mg, 0.16 mmol) was dissolved in dry MeOH (2 mL) and this solution was degassed by bubbling nitrogen through for 5 min (solution A). A suspension of Intermediate 7 (123 mg, 0.176 mmol, 1.1 eq.) and K₂CO₃ (44 mg, 0.321 mmol, 2 eq.) in dry MeOH (2 mL) and THE (2 mL) was degassed by bubbling nitrogen through for 5 min (solution B). Solution A was then added dropwise to solution B and the reaction was stirred at room temperature under nitrogen atmosphere for 1 h. The solvents were evaporated and the residue was dissolved in MeOH (4 mL). p-Toluenesulfonic acid monohydrate (91 mg, 0.481 mmol, 3 eq.) was added. The reaction mixture was stirred at room temperature overnight. The reaction mixture was concentrated under reduced pressure. The residue was dissolved in DCM (20 mL) and washed with saturated aqueous NaHCO₃ (10 mL). The aqueous layer was extracted with DCM (2×10 mL). The combined organic layer was dried over MgSO₄, filtered, and concentrated under reduced pressure. The residue was purified by flash column chromatography on silica gel (24 g, gradient: DCM/MeOH 100/0 to 96/4) to afford Intermediate 25 (70 mg, yield: 61%) as a solid.

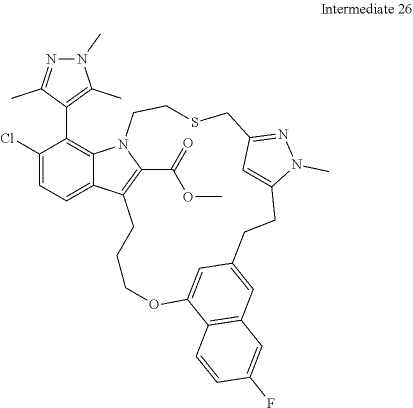

A solution of Intermediate 25 (74 mg, 0.103 mmol) and di-tert-butyl azodicarboxylate (47 mg, 0.206 mmol, 2 eq.) in toluene (2.5 mL) and THF (0.5 mL) was added with a syringe pump (0.1 mL/min) to a solution of triphenylphosphine (54 mg, 0.206 mmol, 2 eq.) in toluene (2.5 mL), stirring at 70° C. Once the addition was complete, the reaction mixture was allowed to cool down to room temperature and volatiles were removed under reduced pressure. The residue was purified by flash column chromatography on silica gel (24 g, gradient: DCM/MeOH 100/0 to 97/3) to afford Intermediate 26 (50 mg, yield: 69%) as a white solid.

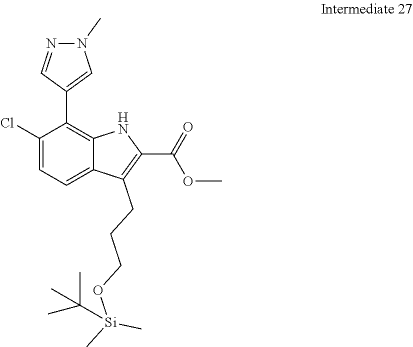 [SMILES: COC(=O)c1[nH]c2c(-c3cnn(C)c3)c(Cl)ccc2c1CCCO[Si](C)(C)C(C)(C)C]

A solution of Intermediate 8 (2180 mg, 4.73 mmol), 1-methyl-4-(4,4,5,5-tetramethyl-1,3,2-dioxaborolan-2-yl)-1H-pyrazole (CAS [761446-44-0], 1083 mg, 5.203 mmol, 1.1 eq.), and Cs₂CO₃ (2312 mg, 7.095 mmol, 1.5 eq.) in water (25 mL) and 1,4-dioxane (100 mL) was prepared and degassed with nitrogen for 5 min. Then, 1,1′-Bis(di-tert-butylphosphino)ferrocene-palladium dichloride (CAS [95408-45-0], 283 mg, 0.473 mmol, 0.1 eq.) was added to the solution and it was further degassed for 5 min. The reaction mixture was stirred at 100° C. for 16 h. The reaction mixture was diluted with EtOAc and water. The layers were separated and the aqueous layer was extracted again with EtOAc. The combined organic layer was washed with brine, dried on MgSO₄, filtered, and evaporated. The residue was purified by column chromatography (Biotage Sfar 100 g; eluent: AcOEt/EtOH 3/1:n-heptane, gradient from 0:100 to 40:60) to give Intermediate 27 (1689 mg, yield: 77%) as a brown solid.

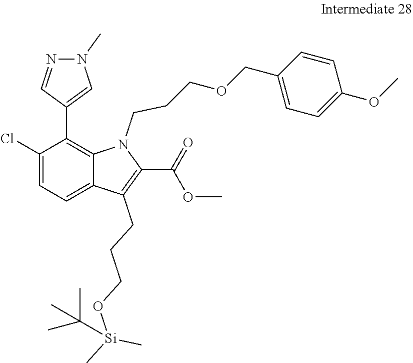 [SMILES: COC(=O)c1c(CCCO[Si](C)(C)C(C)(C)C)c2ccc(Cl)c(-c3cnn(C)c3)c2n1CCCOCc1ccc(OC)cc1]

Cesium carbonate (1784 mg, 5.47 mmol, 1.5 eq.) was added to a solution of Intermediate 27 (1685 mg, 3.647 mmol) and 1-propanol, 3-[(4-methoxyphenyl)methoxy]-, 1-methanesulfonate (CAS [352557-00-7], 1725 mg, 5.47 mmol, 1.5 eq.) in dry DMF (50 mL). The reaction mixture was stirred overnight at 60° C. under nitrogen atmosphere. To push the reaction to completion, the reaction mixture was then stirred at 80° C. for 7 h. After cooling, the reaction mixture was diluted with EtOAc and water. The layers were separated and the aqueous layer was extracted twice with EtOAc. The combined organic layer was washed with brine, dried on MgSO₄, filtered, and the solvent was evaporated. The residue was purified by column chromatography (Biotage Sfar 100 g; eluent: AcOEt/EtOH 3/1:n-heptane, gradient from 0:100 to 40:60) to give Intermediate 28 (2.478 g, 83% pure, yield: 88%) as a black oil.

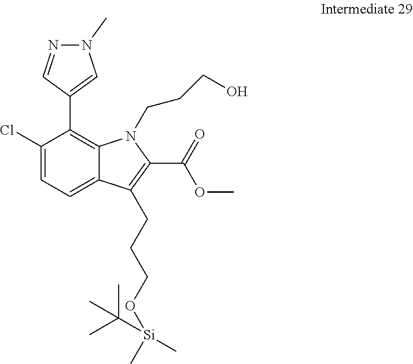 [SMILES: COC(=O)c1c(CCCO[Si](C)(C)C(C)(C)C)c2ccc(Cl)c(-c3cnn(C)c3)c2n1CCCO]

2,3-Dichloro-5,6-cyano-1,4-benzoquinone (1458 mg, 6.424 mmol, 2 eq.) was added to a solution of Intermediate 28 (2478 mg, 3.212 mmol) in DCM (40 mL) and water (5 mL). The reaction mixture was vigorously stirred at room temperature for 1 h. The reaction mixture was diluted with DCM and saturated aqueous NaHCO₃. The layers were separated and the aqueous layer was extracted again with DCM. The combined organic layer was dried on MgSO₄, filtered, and evaporated. The residue was purified by column chromatography (Biotage Sfar 50 g; eluent: AcOEt/EtOH 3/1:n-heptane, gradient from 0:100 to 40:60) to give Intermediate 29 (780 mg, 88% pure, yield: 41%) as a thick brown oil.

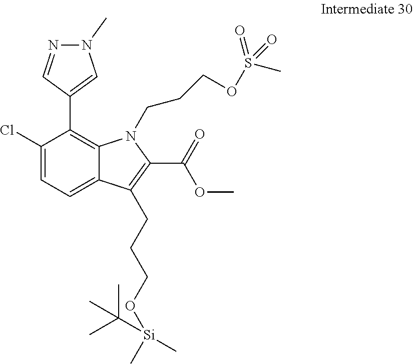 [SMILES: COC(=O)c1c(CCCO[Si](C)(C)C(C)(C)C)c2ccc(Cl)c(-c3cnn(C)c3)c2n1CCCOS(C)(=O)=O]

MsCl (125 μL, 1.584 mmol, 1.2 eq.) was added dropwise to a solution of Intermediate 29 (780 mg, 1.32 mmol) and DIPEA (273 μL, 1.584 mmol, 1.2 eq.) in dry DCM (40 mL) at room temperature under nitrogen atmosphere. The reaction mixture was stirred at room temperature for 2 h. Water was added and the layers were separated. The organic layer was dried on MgSO₄, filtered, and evaporated. The residue was purified by column chromatography (Biotage Sfar 25 g; eluent: AcOEt/EtOH 3/1:n-heptane, gradient from 0:100 to 40:60) to give Intermediate 30 (781 mg, 90% pure, yield: 89%) as a brown oil.

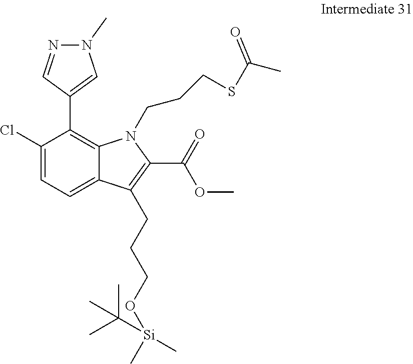 [SMILES: COC(=O)c1c(CCCO[Si](C)(C)C(C)(C)C)c2ccc(Cl)c(-c3cnn(C)c3)c2n1CCCSC(C)=O]

Potassium thioacetate (402 mg, 3.52 mmol, 3 eq.) was added to a solution of Intermediate 30 (780 mg, 1.173 mmol) in dry ACN (30 mL). The reaction mixture was then stirred at 60° C. overnight. The solvent was evaporated and the residue was partitioned between water and EtOAc. The layers were separated and the aqueous layer was extracted again with EtOAc. The combined organic layer was washed with brine, dried on MgSO₄, filtered, and evaporated. The residue was purified by column chromatography (Biotage Sfar 25 g; eluent: AcOEt/EtOH 3/1:n-heptane, gradient from 0:100 to 40:60) to give Intermediate 31 (636 mg, yield: 94%) as a thick yellow oil, dried under vacuum at 50° C.

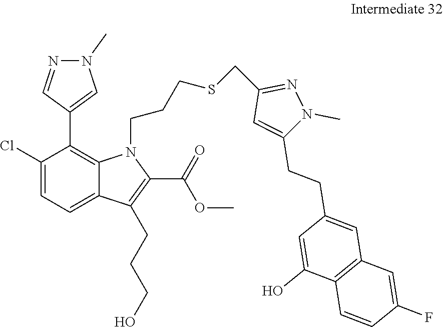 [SMILES: COC(=O)c1c(CCCO)c2ccc(Cl)c(-c3cnn(C)c3)c2n1CCCSCc1cc(CCc2cc(O)c3ccc(F)cc3c2)n(C)n1]

Intermediate 31 (636 mg, 1.1 mmol) was dissolved in dry MeOH (20 mL) and this solution was degassed by bubbling nitrogen through for 5 min (solution A). A suspension of Intermediate 7 (844 mg, 1.21 mmol, 1.1 eq.) and K₂CO₃ (304 mg, 2.2 mmol, 2 eq.) in dry MeOH (30 mL) and THE (10 mL) was degassed by bubbling nitrogen through for 5 min (solution B). Solution B was then added over 5 min to solution A at room temperature under nitrogen atmosphere. The reaction mixture was stirred overnight at room temperature under nitrogen atmosphere. To push the reaction to completion, additional Intermediate 7 (153 mg, 0.22 mmol, 0.2 eq.) was added to the reaction mixture and it was further stirred at room temperature for 1 h. The solvents were evaporated and the residue was dissolved in dry MeOH (100 mL). PTSA·H₂O (1062 mg, 5.498 mmol, 5 eq.) was added to this solution at room temperature. The reaction mixture was stirred at room temperature for 45 min. The solvent was evaporated and the residue was taken up in EtOAc and a saturated aqueous NaHCO₃ solution. The layers were separated and the aqueous layer was extracted again with EtOAc. The combined organic layer was dried on MgSO₄, filtered, and evaporated. The residue was purified by column chromatography (Biotage Sfar 25 g; eluent: AcOEt/EtOH 3/1:n-heptane, gradient from 0:100 to 100:0) followed by preparative SFC (Stationary phase: Chiralpak Daicel ID 20×250 mm, Mobile phase: CO₂, EtOH+0.4% iPrNH₂) to afford Intermediate 32 (188 mg, yield: 24%).

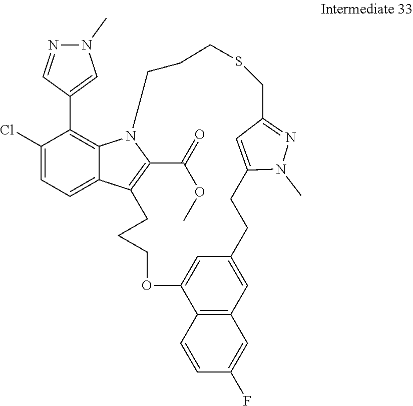 [SMILES: COC(=O)c1c2c3ccc(Cl)c(-c4cnn(C)c4)c3n1CCCCSCc1cc(n(C)n1)CCc1cc(c3ccc(F)cc3c1)OCCC2]

Cyanomethylenetributylphosphorane (CAS [157141-27-0], 266 μL, 1.014 mmol, 4 eq.) was added to a suspension of Intermediate 32 (188 mg, 0.254 mmol) in dry THF (1 mL) and dry ACN (20 mL) at room temperature under nitrogen atmosphere. The reaction mixture was stirred at 80° C. for 2 h under nitrogen atmosphere. To push the reaction to completion, additional cyanomethylenetributylphosphorane (CAS [157141-27-0], 133 μL, 0.507 mmol, 2 eq.) was added to the reaction mixture and it was further stirred at 80° C. for 4 h. The solvent was evaporated and the residue was purified by column chromatography (Biotage Sfar 25 g; eluent: AcOEt/EtOH 3/1:n-heptane 0:100 to 20:80) followed by preparative HPLC (Stationary phase: RP XBridge Prep C18 OBD-10 μm, 30×150 mm, Mobile phase: 0.25% NH₄HCO₃ solution in water, CH₃CN) to afford Intermediate 33 (28 mg, yield: 16%) as an off-white solid.

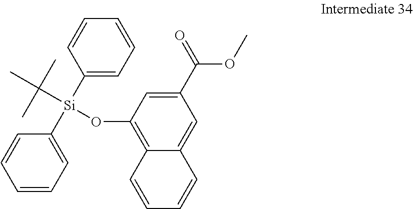 [SMILES: COC(=O)c1cc(O[Si](c2ccccc2)(c2ccccc2)C(C)(C)C)c2ccccc2c1]

TBDPSCl (6.412 mL, 1.25 eq.) was added dropwise to a solution of methyl 4-hydroxy-2-naphthoate (CAS [34205-71-5], 4 g, 19.78 mmol) and imidazole (2.35 g, 1.75 eq.) in DMF (70 mL), cooled to 0° C. Once the addition was complete, the reaction was stirred at room temperature for 14 h. The reaction mixture was diluted with EtOAc (40 mL) and washed subsequently with water, dilute aqueous HCl (0.1 M), saturated aqueous NaHCO₃, and brine (each 30 mL). The organic layer was dried over MgSO₄, filtered, and concentrated. The residue was purified by column chromatography on silica gel (heptane:EtOAc, gradient from 1:0 to 9:1) to afford Intermediate 34 (8.81 g, yield: 91%) as a yellow oil.

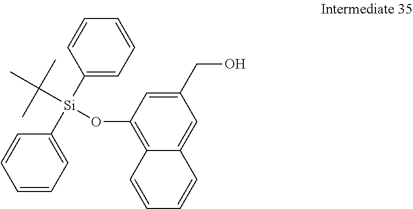 [SMILES: CC(C)(C)[Si](Oc1cc(CO)cc2ccccc12)(c1ccccc1)c1ccccc1]

LiAlH₄ (2 M solution in THF, 9.44 mL, 1.05 eq.) was slowly added to a solution of Intermediate 34 (8.8 g, 17.97 mmol) in THF (70 mL) cooled to 0° C. Once the addition was complete, the reaction mixture was stirred at 0° C. for 30 min. The reaction was quenched by slow addition of EtOAc (20 mL) followed by a saturated solution of Rochelle's salt. The heterogeneous mixture was stirred at room temperature for 2 h. The aqueous layer was extracted with EtOAc (2×65 mL), and the combined organic extracts were washed with brine (20 mL), dried over MgSO₄, filtered, and concentrated. The residue was purified by flash column chromatography on silica gel (heptane:EtOAc,—gradient from 1:0 to 3:1) to give Intermediate 35 (5.81 g, yield: 74%) as a white solid.

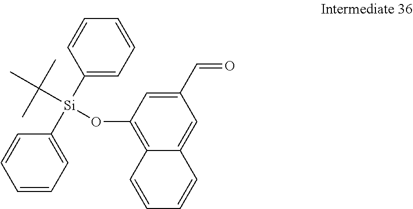 [SMILES: CC(C)(C)[Si](Oc1cc(C=O)cc2ccccc12)(c1ccccc1)c1ccccc1]

MnO₂ (5.81 g, 5 eq.) was added to a solution of Intermediate 35 (5.81 g, 13.38 mmol) in ACN (60 mL) at room temperature. The resulting solution was stirred at 60° C. for 2 h. The reaction mixture was filtered over a pad of Dicalite® and concentrated to give Intermediate 36 (5.47 g, yield: 94%) as a white solid, used without further purification.

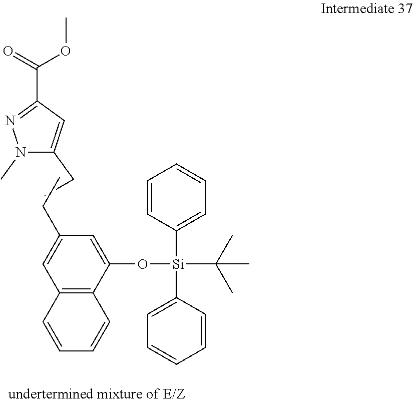

NaH (653 mg, 1.1 eq.) was added to a suspension of ((3-(methoxycarbonyl)-1-methyl-1H-pyrazol-5-yl)methyl)triphenylphosphonium chloride (CAS [2245716-31-6], 8.094 g, 1.1 eq.) in THE (90 mL) at 0° C. The resulting solution (solution A) was stirred at 0° C. for 45 min before it was cooled to −25° C. A solution of Intermediate 36 (6.7 g, 15.5 mmol) in THE (16 mL) was added slowly to solution A while maintaining the temperature between −20° C. and −30° C. Once the addition was complete, the reaction mixture was stirred at −10° C. for 1 h. The reaction was quenched by slow addition of saturated aqueous NH₄C₁ (10 mL) at −10° C. and was diluted with EtOAc (100 mL). The layers were separated and the aqueous layer was extracted with EtOAc (2×100 mL). The combined organic layer was dried over MgSO₄, filtered, and concentrated. The residue was purified by column chromatography on silica gel (heptane:EtOAc, gradient from 1:0 to 7:3) to afford Intermediate 37 (6.75 g, yield: 75%) as a white foam.

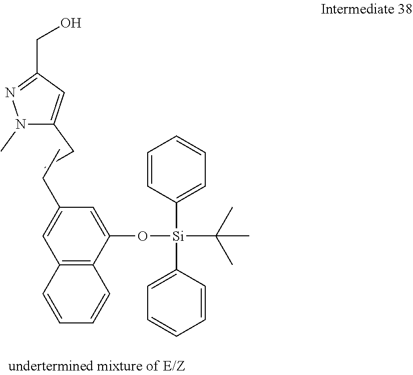

LiAlH₄ (2 M solution in THF, 6.1 mL, 1.05 eq.) was slowly added to a solution of Intermediate 37 (6.7 g, 11.64 mmol) in THE (45 mL) cooled to 0° C. Once the addition was complete, the reaction mixture was stirred at 0° C. for 30 min. The reaction was quenched by slow addition of EtOAc (20 mL) followed by a saturated solution of Rochelle's salt. The heterogeneous mixture was stirred at room temperature for 2 h. The aqueous layer was extracted with EtOAc (2×65 mL), and the combined organic extracts were washed with brine (20 mL), dried over MgSO₄, filtered, and concentrated to afford Intermediate 38 (6.01 g, yield: 94%) as a white foam, used without further purification.

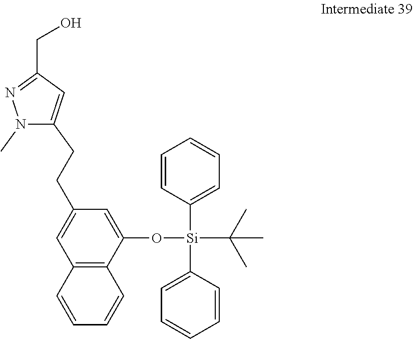 [SMILES: Cn1nc(CO)cc1CCc1cc(O[Si](c2ccccc2)(c2ccccc2)C(C)(C)C)c2ccccc2c1]

Intermediate 38 (5.95 g, 10.89 mmol) was dissolved in MeOH (280 mL). Pd/C (10%, 1159 mg, 0.1 eq.) was added under nitrogen atmosphere. The reaction mixture was then flushed with hydrogen gas and vacuum (3 times), then hydrogen (atmospheric pressure, 244 mL, 1 eq.) was taken up while stirring at room temperature. The reaction mixture was filtered over a pad of Dicalite® and concentrated to give Intermediate 39 (5.9 g, yield: 98%) as a glassy yellow solid, used without further purification.

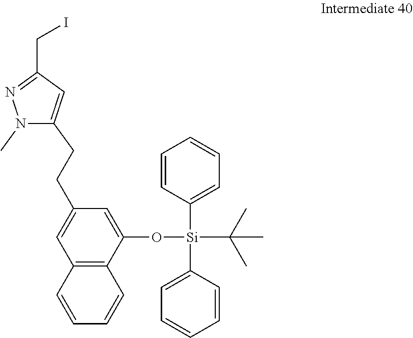 [SMILES: Cn1nc(CI)cc1CCc1cc(O[Si](c2ccccc2)(c2ccccc2)C(C)(C)C)c2ccccc2c1]

Methanesulfonic anhydride (517 mg, 2.966 mmol, 2 eq.), followed by DIPEA (511 μL, 2.966 mmol, 2 eq.), were added to a solution of Intermediate 39 (813 mg, 1.483 mmol) in dry ACN (40 mL) and dry THE (20 mL) at room temperature. The reaction mixture was stirred at room temperature for 2 h. Sodium iodide (1112 mg, 7.416 mmol, 5 eq.) was then added to the reaction mixture and it was stirred at room temperature overnight. The solvents were evaporated and the residue was partitioned between aqueous Na₂S₂O₃ and EtOAc. The layers were separated and the organic layer was dried on MgSO₄, filtered, and evaporated to give Intermediate 40 (964 mg, yield: 90%), dried under vacuum at 50° C., and used without further purification.

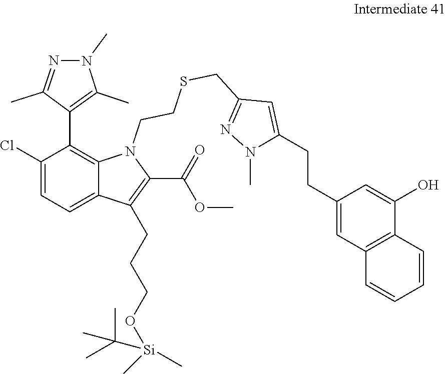 [SMILES: COC(=O)c1c(CCCO[Si](C)(C)C(C)(C)C)c2ccc(Cl)c(-c3c(C)nn(C)c3C)c2n1CCSCc1cc(CCc2cc(O)c3ccccc3c2)n(C)n1]

Intermediate 24 (600 mg, 1.013 mmol) was dissolved in dry MeOH (15 mL) and this solution was degassed by bubbling nitrogen through for 5 min (solution A). A suspension of Intermediate 40 (820 mg, 1.145 mmol, 1.1 eq.) and K₂CO₃ (280 mg, 2.026 mmol, 2 eq.) in dry MeOH (15 mL) and THE (15 mL) was degassed by bubbling nitrogen through for 5 min (solution B). Solution A was then added to solution B at room temperature under nitrogen atmosphere. The reaction mixture was stirred for 3 days at room temperature under nitrogen atmosphere. The solvents were evaporated and the residue was partitioned between water and EtOAc. The layers were separated and the aqueous layer was extracted again with EtOAc. The combined organic layer was dried on MgSO₄, filtered, and evaporated to give Intermediate 41 (1335 mg, yield: quantitative) as a thick brown oil, dried under vacuum at 50° C., and used without further purification.

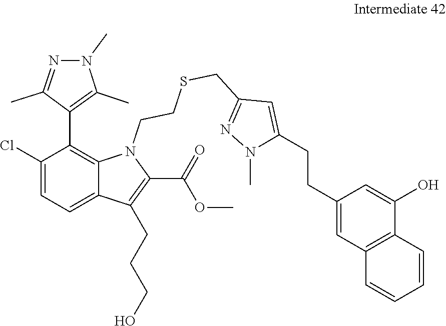 [SMILES: COC(=O)c1c(CCCO)c2ccc(Cl)c(-c3c(C)nn(C)c3C)c2n1CCSCc1cc(CCc2cc(O)c3ccccc3c2)n(C)n1]

PTSA·H₂O (1203 mg, 6.228 mmol, 5 eq.) was added to a solution of Intermediate 41 (1335 mg, 76% pure, 1.335 mmol) in dry MeOH (100 mL) at room temperature. The reaction mixture was stirred at room temperature for 2.5 h. The solvent was evaporated and the residue was taken up in EtOAc and a saturated aqueous NaHCO₃ solution. The layers were separated and the aqueous layer was extracted again with EtOAc. The combined organic layer was dried on MgSO₄, filtered, and evaporated. The residue was purified by column chromatography (Biotage Sfar 50 g; eluent: AcOEt/EtOH 3/1:n-heptane, gradient from 0:100 to 100:0) to give Intermediate 42 (485 mg, 56%) as a white solid, dried under vacuum at 50° C.

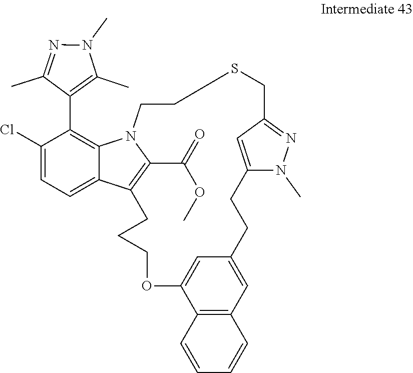

A solution of cyanomethylenetributylphosphorane (CAS [157141-27-0], 178 μL, 0.678 mmol, 1 eq.) in dry ACN (45 mL) was stirred at 80° C. under nitrogen atmosphere (solution A). A solution of Intermediate 42 (475 mg, 0.678 mmol) and cyanomethylenetributylphosphorane (CAS [157141-27-0], 711 μL, 2.713 mmol, 4 eq.) in THE (2 mL) and ACN (8 mL) was then added dropwise via syringe pump (0.1 mL/min) to solution A while stirring at 80° C. under nitrogen atmosphere. After the addition was complete, the reaction mixture was stirred for 1 h at 80° C. under nitrogen atmosphere. The solvent was evaporated and the residue was purified by column chromatography (Biotage Sfar 25 g; eluent: AcOEt/EtOH 3/1:n-heptane, gradient from 0:100 to 100:0) to give Intermediate 43 (268 mg, yield: 58%) as a light brown solid.

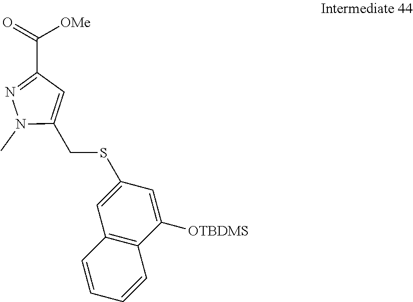 [SMILES: COC(=O)c1cc(CSc2cc(O[Si](C)(C)C(C)(C)C)c3ccccc3c2)n(C)n1]

Imidazole (258 mg, 1.4 eq.) was added to a solution of methyl 5-(((4-hydroxynaphthalen-2-yl)thio)methyl)-1-methyl-1H-pyrazole-3-carboxylate (CAS [2245716-34-9], 890 mg) and TBDMSCl (511 mg, 1.25 eq.) in DMF (17.8 mL). The reaction was stirred at room temperature for 48 h. The reaction mixture was diluted with EtOAc (100 mL) and water (50 mL). The organic layer was separated and washed with brine (2×50 mL). The combined aqueous layers were extracted with EtOAc (50 mL). The combined organic layers were dried over MgSO₄, filtered and evaporated. The crude mixture was purified by flash chromatography on silica gel (40 g, gradient: heptane/EtOAc 100/0 to 60/40) to obtain Intermediate 44 (1.24 g, quant.).

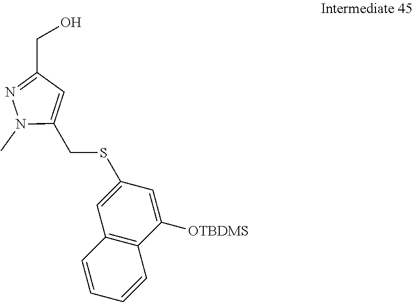 [SMILES: Cn1nc(CO)cc1CSc1cc(O[Si](C)(C)C(C)(C)C)c2ccccc2c1]

DIBALH (1 M in heptane, 3.49 mL, 3.490 mmol, 1.5 eq.) was added dropwise to a solution of Intermediate 44 (1030 mg, 2.327 mmol) in THF (40 mL) at 0° C. under nitrogen atmosphere and the reaction mixture was stirred at 0° C. for 30 min. Additional DIBALH (1 M in heptane, 2.33 mL, 2.327 mmol, 1 eq.) was added and the reaction mixture was further stirred for 10 min. The reaction was treated with wet THF (40 mL) and, after a few min stirring, with water (10 mL, initial dropwise addition). The mixture was allowed to warm up to room temperature and then Celite® was added. After 5 min stirring, the mixture was filtered, washing with EtOAc. The filtrate was dried with MgSO₄, filtered, and evaporated to give Intermediate 45 (892 mg, 92%) as a colorless paste that solidified upon standing, and was used without further purification.

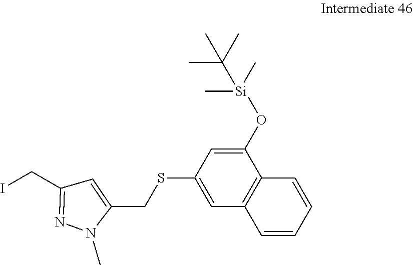 [SMILES: Cn1nc(CI)cc1CSc1cc(O[Si](C)(C)C(C)(C)C)c2ccccc2c1]

Intermediate 46 was prepared according to an analogous procedure as for Intermediate 40, using Intermediate 45 instead of Intermediate 39.

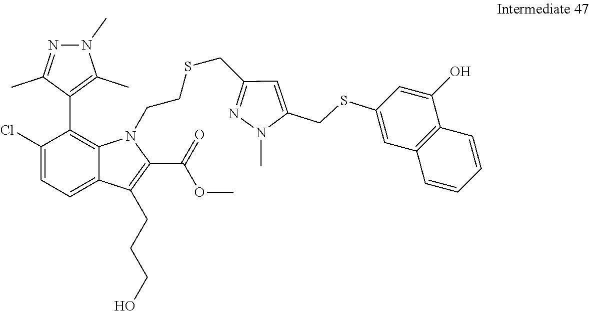 [SMILES: COC(=O)c1c(CCCO)c2ccc(Cl)c(-c3c(C)nn(C)c3C)c2n1CCSCc1cc(CSc2cc(O)c3ccccc3c2)n(C)n1]

Intermediate 47 was prepared in two steps according to an analogous procedure as for Intermediate 42, starting from Intermediate 46 instead of Intermediate 40.

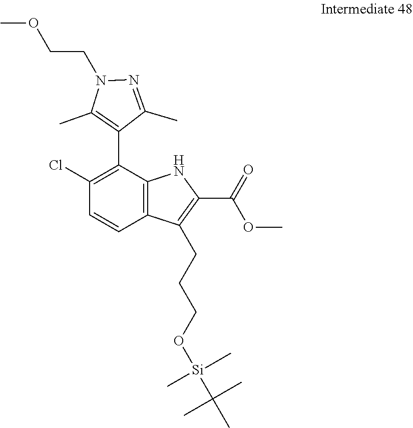

A solution of 1-(2-methoxyethyl)-3,5-dimethyl-4-(4,4,5,5-tetramethyl-1,3,2-dioxaborolan-2-yl)-1H-pyrazole (CAS [1200537-40-1], 1003 mg, 3.58 mmol, 1.5 eq.), Intermediate 8 (1.1 g, 2.387 mmol), and Na₂CO₃ (759 mg, 7.16 mmol, 3 eq.) in 1,4-dioxane (10 mL) and water (1 mL) was degassed with nitrogen for 2 min. CPhos Pd G3 (CAS [1447963-73-6], 385 mg, 0.477 mmol, 0.2 eq.) was then added. The vial was sealed and the reaction mixture was stirred at 60° C. for 3 h in a microwave oven. After cooling, the mixture was filtered over dicalite, concentrated in vacuo, and purified by column chromatography on silica gel (Biotage Sfar 50 g, n-heptane/(EtOAc/EtOH (3:1)), gradient from 100/0 to 80/20) to give Intermediate 48 (1.8 g, yield: quantitative) as an oil.

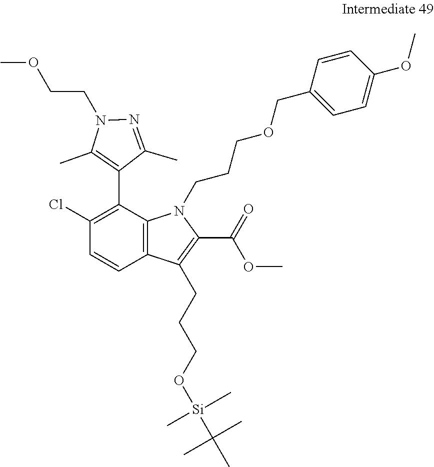

A solution of Intermediate 48 (1.8 g, 2.999 mmol), 1-((3-iodopropoxy)methyl)-4-methoxybenzene (CAS [198411-17-5], 1377 mg, 4.499 mmol, 1.5 eq.), and Cs₂CO₃ (1563 mg, 4.798 mmol, 1.6 eq.) in dry DMF (23 mL), was stirred at 60° C. for 2 h. After cooling, the reaction mixture was diluted with EtOAc and water. The layers were separated and the aqueous layer was extracted again with EtOAc. The combined organic layer was washed with brine, dried on MgSO₄, filtered, and the solvent was evaporated. The residue was purified by column chromatography (Biotage Sfar 25 g; eluent: (AcOEt/EtOH (3:1))/n-heptane, gradient from 0:100 to 40:60) to give Intermediate 49 (2.267 g, 75% pure, yield: 79%), as a dark oil.

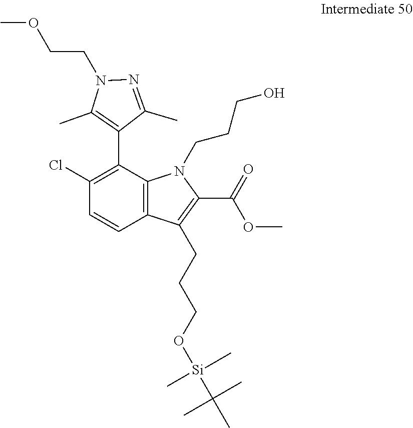

DDQ (813 mg, 3.58 mmol, 1.5 eq.) was added to a solution of Intermediate 49 (2.267 g, 2.387 mmol) in DCM (46 mL) and water (4 mL). The reaction mixture was stirred at room temperature for 1 h. Saturated aqueous NaHCO₃ (20 mL) was added and the reaction mixture was stirred vigorously for 5 min. The reaction mixture was then diluted with DCM and the layers were separated. The organic layer was washed with brine, dried on MgSO₄, filtered, and evaporated. The residue was purified by column chromatography (Biotage Sfar 200 g; eluent: n-heptane/(EtOAc/EtOH (3:1)), gradient from 100:0 to 60:40) to give Intermediate 50 (1.038 g, yield: 72%) as a light brown oil.

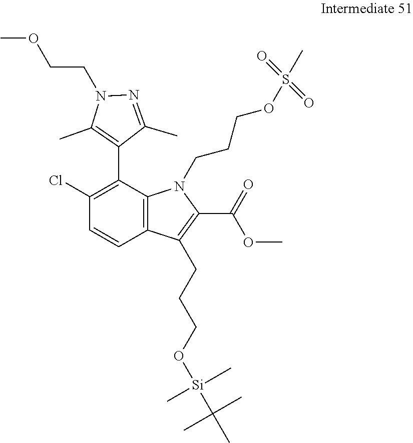 [SMILES: COCCn1nc(C)c(-c2c(Cl)ccc3c(CCCO[Si](C)(C)C(C)(C)C)c(C(=O)OC)n(CCCOS(C)(=O)=O)c23)c1C]

MsCl (0.16 mL, 2.061 mmol, 1.2 eq.) was added dropwise to a solution of Intermediate 50 (1.038 g, 1.718 mmol) and DIPEA (0.444 mL, 2.576 mmol, 1.5 eq.) in dry DCM (11 mL) at room temperature. The reaction mixture was stirred at room temperature for 1 h. The solvent was evaporated and the residue was purified by column chromatography on silica gel (Biotage Sfar 10 g; eluent: (AcOEt/EtOH (3:1)/n-heptane, gradient from 0:100 to 40:60) to give Intermediate 51 (1.03 g, yield: 85%) as a thick colorless oil.

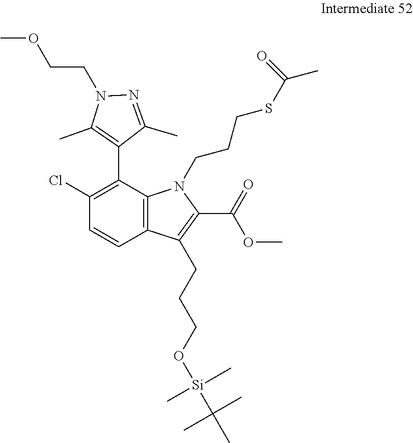 [SMILES: COCCn1nc(C)c(-c2c(Cl)ccc3c(CCCO[Si](C)(C)C(C)(C)C)c(C(=O)OC)n(CCCSC(C)=O)c23)c1C]

Potassium thioacetate (526 mg, 4.61 mmol, 3 eq.) was added to a solution of Intermediate 51 (1.03 g, 1.537 mmol) in dry ACN (12 mL). The reaction mixture was stirred at 60° C. for 16 h. The solvent was evaporated and the residue was partitioned between water and EtOAc. The layers were separated and the aqueous layer was extracted again with EtOAc. The combined organic layer was dried with MgSO₄, and evaporated. The residue was purified by column chromatography on silica gel (SNAP Ultra 25 g; eluent: (AcOEt/EtOH (3:1))/n-heptane, gradient from 0:100 to 40:60) to give Intermediate 52 (800 mg, yield: 80%) as a thick brown oil.

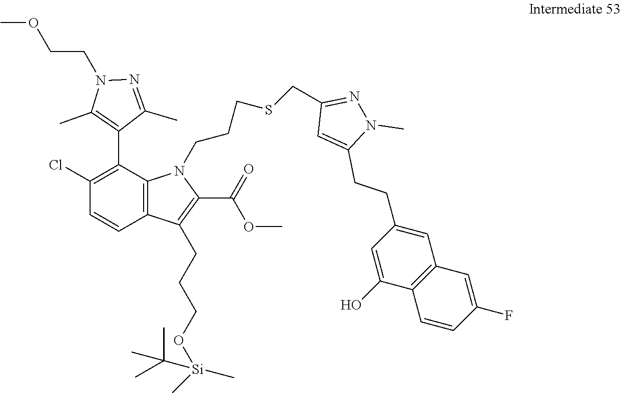 [SMILES: COCCn1nc(C)c(-c2c(Cl)ccc3c(CCCO[Si](C)(C)C(C)(C)C)c(C(=O)OC)n(CCCSCc4cc(CCc5cc(O)c6ccc(F)cc6c5)n(C)n4)c23)c1C]

Intermediate 52 (800 mg, 1.23 mmol) was dissolved in MeOH (10 mL) and this solution was degassed with nitrogen for 5 min (solution A). A suspension of Intermediate 7 (878 mg, 1.353 mmol, 1.1 eq.) and K₂CO₃ (510 mg, 3.69 mmol, 3 eq.) in dry MeOH (10 mL) and THE (5.5 mL) was degassed with nitrogen for 5 min (solution B). Solution A was then added dropwise to solution B at room temperature under nitrogen atmosphere. The reaction mixture was stirred at room temperature under nitrogen atmosphere for 2 h. The solvents were evaporated and the residue was purified by column chromatography on silica gel (SNAP Ultra 25 g; eluent: (AcOEt/EtOH (3:1))/n-heptane, gradient from 0:100 to 40:60) to give Intermediate 53 (1.07 g, yield: 98%) as a brown solid.

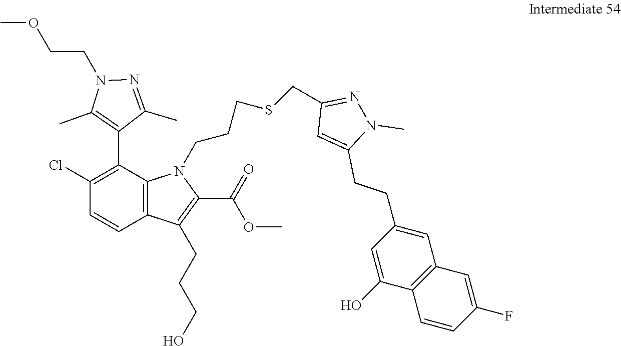 [SMILES: COCCn1nc(C)c(-c2c(Cl)ccc3c(CCCO)c(C(=O)OC)n(CCCSCc4cc(CCc5cc(O)c6ccc(F)cc6c5)n(C)n4)c23)c1C]

Intermediate 53 (1 g, 1.123 mmol) was dissolved in MeOH (4.5 mL) and PTSA·H₂O (854 mg, 4.491 mmol, 4 eq.) was added. The reaction mixture was stirred at room temperature for 1 h. The solvent was evaporated and the residue was redissolved in EtOAc and washed with saturated aqueous NaHCO₃. The organic layer was dried with MgSO₄, filtered, and concentrated in vacuo to give Intermediate 54 (900 mg, 95% pure, yield: 98%), used without further purification.

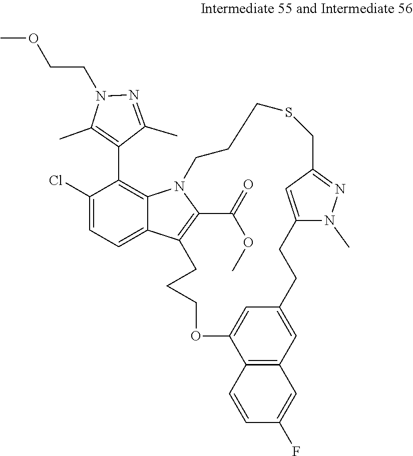

### Intermediate 55: Sₐ or Rₐ; One Atropisomer but Absolute Stereochemistry Undetermined

### Intermediate 56: Rₐ or Sₐ; One Atropisomer but Absolute Stereochemistry Undetermined

(Tributylphosphoranylidene)acetonitrile (CAS [157141-27-0], 0.975 mL, 3.72 mmol, 4 eq.) was added to a solution of Intermediate 54 (760 mg, 0.93 mmol)) in dry ACN (5 mL) at room temperature under nitrogen atmosphere. The reaction mixture was stirred at 80° C. for 2 h under nitrogen atmosphere. The solution was concentrated in vacuo and the residue was purified by column chromatography on silica gel (Biotage Sfar 25 g, n-heptane/(AcOEt/EtOH (3:1)), gradient from 100/0 to 20/80), followed by preparative SFC (Stationary phase: Chiralpak Diacel AD 20×250 mm, Mobile phase: CO₂, EtOH+0.4% iPrNH₂) to give Intermediate 55 (65 mg, yield: 9%) and Intermediate 56 (55 mg, yield: 8%).

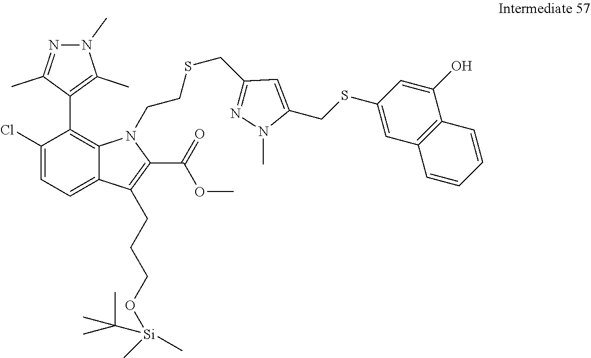 [SMILES: COC(=O)c1c(CCCO[Si](C)(C)C(C)(C)C)c2ccc(Cl)c(-c3c(C)nn(C)c3C)c2n1CCSCc1cc(CSc2cc(O)c3ccccc3c2)n(C)n1]

Intermediate 24 (600 mg, 1.013 mmol) was dissolved in dry MeOH (15 mL) and this solution was degassed by bubbling nitrogen through for 5 min (solution A). A suspension of Intermediate 46 (727 mg, 1.165 mmol, 1.15 eq.) and K₂CO₃ (280 mg, 2.026 mmol, 2 eq.) in dry MeOH (15 mL) and THF (15 mL) was degassed by bubbling nitrogen through for 5 min (solution B). Solution A was then added to solution B at room temperature under nitrogen atmosphere. The reaction mixture was stirred overnight at room temperature under nitrogen atmosphere. The solvents were evaporated and the residue was partitioned between water and EtOAc. The layers were separated and the aqueous layer was extracted again with EtOAc. The combined organic layer was dried on MgSO₄, filtered, and evaporated to give Intermediate 57 (829 mg, 74% pure, yield: 73%) as a brown solid, dried under vacuum at 50° C., and used without further purification.

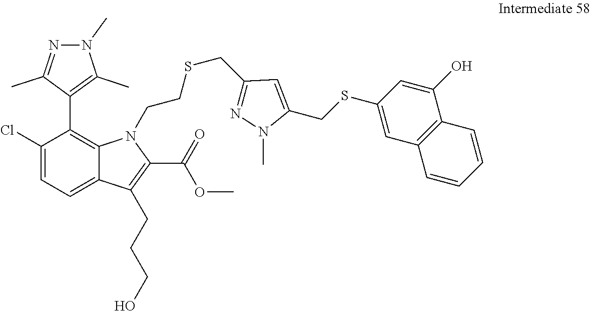 [SMILES: COC(=O)c1c(CCCO)c2ccc(Cl)c(-c3c(C)nn(C)c3C)c2n1CCSCc1cc(CSc2cc(O)c3ccccc3c2)n(C)n1]

PTSA·H₂O (711 mg, 3.684 mmol, 5 eq.) was added to a solution of Intermediate 57 (829 mg, 74% pure, 0.737 mmol) in dry MeOH (100 mL) at room temperature. The reaction mixture was stirred at room temperature for 2.5 h. The solvent was evaporated and the residue was taken up in EtOAc and a saturated aqueous NaHCO₃ solution. The layers were separated and the aqueous layer was extracted again with EtOAc. The combined organic layer was dried on MgSO₄, filtered, and evaporated. The residue was purified by column chromatography on silica gel (Biotage Sfar 50 g; eluent: (AcOEt/EtOH (3:1))/n-heptane, gradient from 0:100 to 100:0) to give Intermediate 58 (330 mg, yield: 62%) as a white solid, dried under vacuum at 50° C.

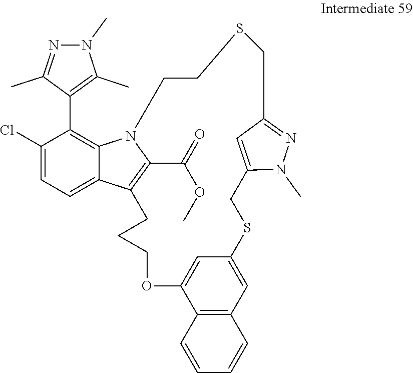

Intermediate 59 was prepared according to an analogous procedure as for Intermediate 43, using Intermediate 58 instead of Intermediate 42.

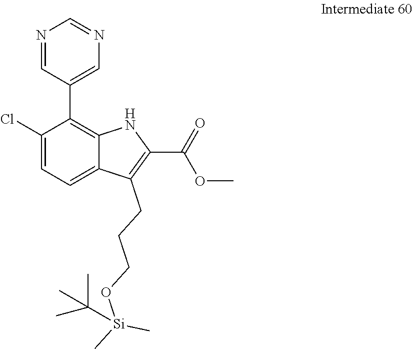 [SMILES: COC(=O)c1[nH]c2c(-c3cncnc3)c(Cl)ccc2c1CCCO[Si](C)(C)C(C)(C)C]

A solution of Intermediate 8 (2873 mg, 6.234 mmol), pyrimidine-5-boronic acid (773 mg, 6.234 mmol, 1 eq.), and Cs₂CO₃ (3047 mg, 9.351 mmol, 1.5 eq.) in water (30 mL) and 1,4-dioxane (150 mL) was prepared and degassed with nitrogen for 5 min. 1,1′-Bis(di-tert-butylphosphino)ferrocene-palladium dichloride (CAS [95408-45-0], 373 mg, 0.623 mmol, 0.1 eq.) was then added to the solution and it was further degassed for 5 min. The reaction mixture was stirred at 100° C. for 2.5 h. After cooling, the reaction mixture was diluted with water and EtOAc. The layers were separated and the aqueous layer was extracted twice with EtOAc. The combined organic layer was washed with brine, dried on MgSO₄, filtered and evaporated. The residue was purified by column chromatography on silica gel (Biotage Sfar 100 g; eluent: (AcOEt/EtOH (3:1))/n-heptane, gradient from 0:100 to 40:60) to give Intermediate 60 (2102 mg, yield: 73%) as a black solid.

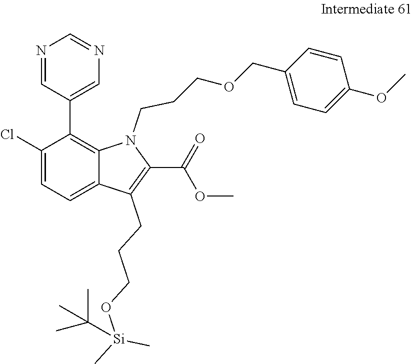 [SMILES: COC(=O)c1c(CCCO[Si](C)(C)C(C)(C)C)c2ccc(Cl)c(-c3cncnc3)c2n1CCCOCc1ccc(OC)cc1]

Cesium carbonate (488 mg, 1.496 mmol, 1.5 eq.) was added to a solution of Intermediate 60 (459 mg, 0.998 mmol) and 1-((3-iodopropoxy)methyl)-4-methoxybenzene (CAS [198411-17-5], 458 mg, 1.496 mmol, 1.5 eq.) in dry DMF (20 mL) at room temperature. The reaction mixture was stirred at 60° C. under nitrogen for 2.5 h. The mixture was diluted with EtOAc and water. The layers were separated and the aqueous layer was extracted again with EtOAc. The combined organic layer was dried on MgSO₄, filtered, and the solvent was evaporated. The residue was purified by column chromatography on silica gel (Biotage Sfar 100 g; eluent: (AcOEt/EtOH (3:1))/n-heptane, gradient from 0:100 to 40:60) to give Intermediate 61 (887 mg, 40% pure, yield: 56%) as a brown oil, dried under vacuum at 50° C., and used without further purification.

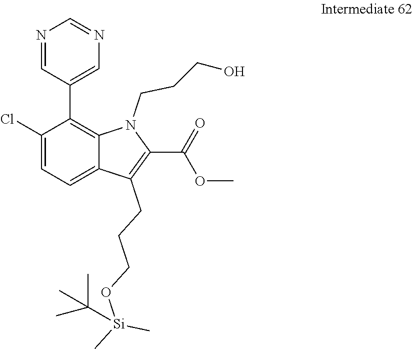 [SMILES: COC(=O)c1c(CCCO[Si](C)(C)C(C)(C)C)c2ccc(Cl)c(-c3cncnc3)c2n1CCCO]

DDQ (252 mg, 1.112 mmol, 2 eq.) was added to a solution of Intermediate 61 (887 mg, 0.556 mmol) in DCM (40 mL) and water (4 mL). The reaction mixture was vigorously stirred at room temperature for 2 h. More DDQ (126 mg, 0.556 mmol, 1 eq.) was added and stirring was continued at room temperature overnight. Again, more DDQ (252 mg, 1.112 mmol, 2 eq.) was added and stirring was continued at room temperature for 3 h. The reaction mixture was diluted with DCM and saturated aqueous NaHCO₃. The layers were separated and the aqueous layer was extracted again with DCM. The combined organic layer was dried on MgSO₄, filtered, and evaporated. The residue was purified by column chromatography on silica gel (Biotage Sfar 50 g; eluent: (AcOEt/EtOH (3:1))/n-heptane, gradient from 0:100 to 40:60) to give Intermediate 62 (200 mg, 60% pure, yield: 42%) as a light-brown solid, used without further purification.

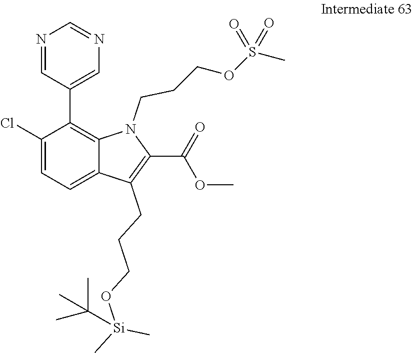 [SMILES: COC(=O)c1c(CCCO[Si](C)(C)C(C)(C)C)c2ccc(Cl)c(-c3cncnc3)c2n1CCCOS(C)(=O)=O]

Methanesulfonic anhydride (60 mg, 0.347 mmol, 1.5 eq.) and DIPEA (80 μL, 0.463 mmol, 2 eq.) were added to a solution of Intermediate 62 (200 mg, 0.232 mmol) in dry DCM (10 mL) at room temperature. The reaction mixture was stirred overnight at room temperature. More methanesulfonic anhydride (30 mg, 0.173 mmol, 0.7 eq.) was added and the reaction mixture was stirred at room temperature for 1 h. Water was added and the layers were separated. The aqueous layer was extracted with DCM. The combined organic layer was dried by filtration on Extrelut NT3 (Supelco®), and evaporated to give Intermediate 63 (254 mg, 68% pure, quantitative) as a brown oil, dried under vacuum at 50° C., and used without further purification.

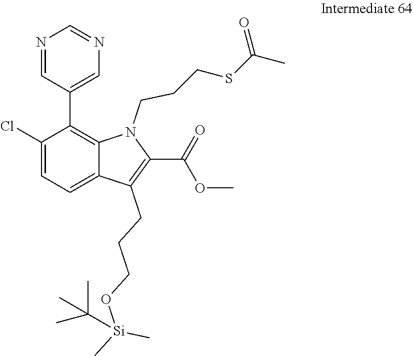 [SMILES: COC(=O)c1c(CCCO[Si](C)(C)C(C)(C)C)c2ccc(Cl)c(-c3cncnc3)c2n1CCCSC(C)=O]

Potassium thioacetate (98 mg, 0.855 mmol, 3 eq.) was added to a solution of Intermediate 63 (68% pure, 250 mg, 0.285 mmol) in dry ACN (20 mL). The reaction mixture was then stirred at 60° C. overnight. The solvent was evaporated and the residue was partitioned between water and EtOAc. The layers were separated and the aqueous layer was extracted again with EtOAc. The combined organic layer was dried by filtration on Extrelut NT3 (Supelco®), and evaporated. The residue was purified by column chromatography on silica gel (Biotage Sfar 10 g; eluent: (AcOEt/EtOH (3:1))/n-heptane, gradient from 0:100 to 40:60) to give Intermediate 64 (179 mg, 87% pure, yield: 95%) as a thick brown oil.

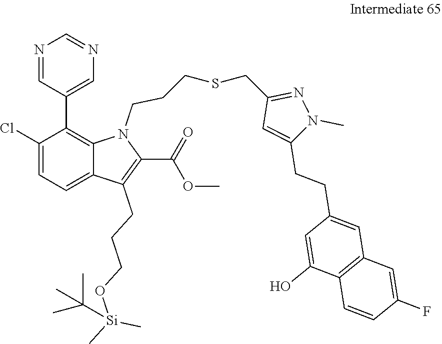 [SMILES: COC(=O)c1c(CCCO[Si](C)(C)C(C)(C)C)c2ccc(Cl)c(-c3cncnc3)c2n1CCCSCc1cc(CCc2cc(O)c3ccc(F)cc3c2)n(C)n1]

Intermediate 64 (179 mg, 0.27 mmol) was dissolved in dry MeOH (4 mL) and this solution was degassed by bubbling nitrogen through for 5 min (solution A). A suspension of Intermediate 7 (193 mg, 0.297 mmol, 1.1 eq.) and K₂CO₃ (75 mg, 0.541 mmol, 2 eq.) in dry MeOH (4 mL) and THF (4 mL) was degassed by bubbling nitrogen through for 5 min (solution B). Solution A was then added to solution B at room temperature under nitrogen atmosphere. The reaction mixture was stirred for 3 days at room temperature under nitrogen atmosphere. The solvents were evaporated and the residue was partitioned between water and EtOAc. The layers were separated and the aqueous layer was extracted again with EtOAc. The combined organic layer was dried by filtration on Extrelut NT3 (Supelco®) and evaporated to give Intermediate 65 (284 mg, 74% pure, yield: 95%) as a thick brown oil, dried under vacuum at 50° C., and used without further purification.

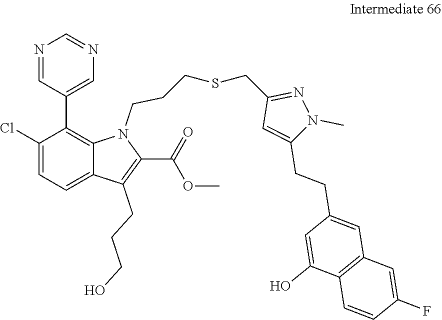 [SMILES: COC(=O)c1c(CCCO)c2ccc(Cl)c(-c3cncnc3)c2n1CCCSCc1cc(CCc2cc(O)c3ccc(F)cc3c2)n(C)n1]

PTSA·H₂O (249 mg, 1.287 mmol, 5 eq.) was added to a solution of Intermediate 65 (284 mg, 74% pure, 0.257 mmol) in dry MeOH (20 mL) at room temperature. The reaction mixture was stirred at room temperature for 2.5 h. The solvent was evaporated and the residue was taken up in EtOAc and a saturated aqueous NaHCO₃ solution. The layers were separated and the aqueous layer was extracted again with EtOAc. The combined organic layer was dried by filtration on Extrelut NT3 and evaporated. The residue was purified by column chromatography on silica gel (Biotage Sfar 10 g; eluent: (AcOEt/EtOH (3:1))/n-heptane, gradient from 0:100 to 100:0) to give Intermediate 66 (120 mg, 80% pure, yield: 53%) as a brown solid.

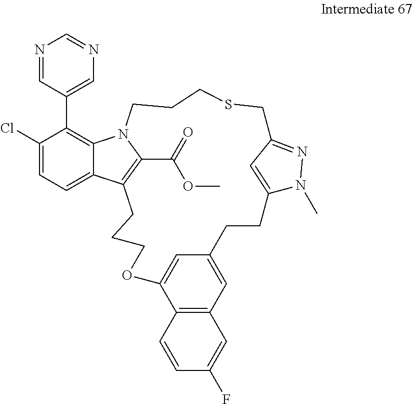 [SMILES: COC(=O)c1c2c3ccc(Cl)c(-c4cncnc4)c3n1CCCSCc1cc(n(C)n1)CCc1cc(c3ccc(F)cc3c1)OCCC2]

Intermediate 67 was prepared according to an analogous procedure as for Intermediate 43, using Intermediate 66 instead of Intermediate 42.

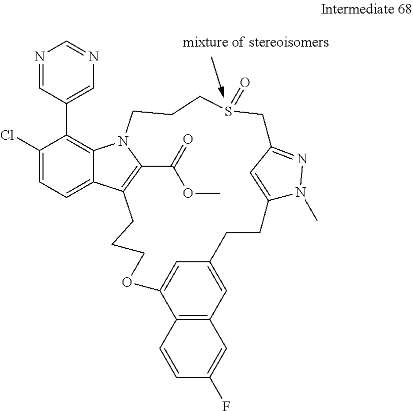 [SMILES: COC(=O)c1c2c3ccc(Cl)c(-c4cncnc4)c3n1CCCS(=O)Cc1cc(n(C)n1)CCc1cc(c3ccc(F)cc3c1)OCCC2]

m-CPBA (11 mg, 0.062 mmol, 1.1 eq.) was added in one portion to a solution of Intermediate 67 (39 mg, 0.057 mmol) in DCM (10 mL) at room temperature. The reaction mixture was stirred for 1 h at room temperature. Aqueous Na₂CO₃ was added to the reaction mixture and the layers were separated. The aqueous layer was extracted again with DCM. The combined organic layer was dried by filtration on Extrelut NT3 (Supelco®), and evaporated. The residue was purified by column chromatography on silica gel (Biotage Sfar 10 g; eluent: DCM/MeOH, gradient from 100:0 to 0:100) to give Intermediate 68 (31 mg, yield: 78%) as a white solid.

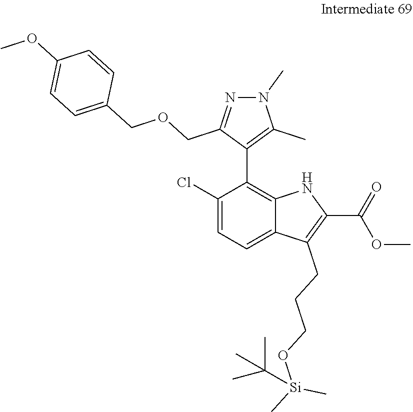

1,1′-Bis(di-tert-butylphosphino)ferrocene-palladium dichloride (CAS [95408-45-0], 265 mg, 0.443 mmol, 0.1 eq.) was added to a red-brown solution of Intermediate 8 (2043 mg, 4.433 mmol), 3-[(4-methoxyphenyl)methoxymethyl]-1,5-dimethyl-4-(4,4,5,5-tetramethyl-1,3,2-dioxaborolan-2-yl)pyrazole (CAS [2143010-90-4], 1815 mg, 4.876 mmol, 1.1 eq.), and Cs₂CO₃ (2166 mg, 6.649 mmol, 1.5 eq.), in 1,4-dioxane (30 mL) and water (6 mL). The solution was degassed with nitrogen for 5 min. The vial was sealed and stirred at 90° C. for 2 h in a microwave oven. The reaction mixture was diluted with water and EtOAc. The layers were separated and the organic layer was washed with water. The combined aqueous layer was extracted again with EtOAc. The combined organic layer was dried on MgSO₄, filtered, and evaporated. The residue was purified by column chromatography on silica gel (Biotage SNAP Ultra 50 g; eluent: n-heptane/(AcOEt/EtOH (3:1)), gradient from 100:0 to 50:50) to give Intermediate 69 (3158 mg, quantitative) as a black oil.

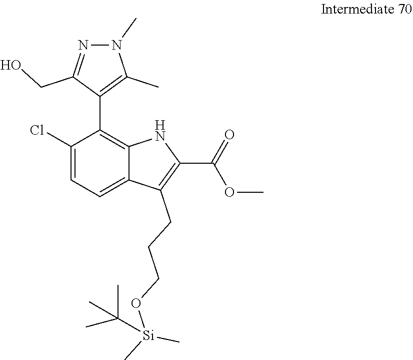 [SMILES: COC(=O)c1[nH]c2c(-c3c(CO)nn(C)c3C)c(Cl)ccc2c1CCCO[Si](C)(C)C(C)(C)C]

DDQ (2289 mg, 10.085 mmol, 2 eq.) was added to a solution of Intermediate 69 (3158 mg, 5.043 mmol) in DCM (120 mL) and water (12 mL). The reaction mixture was vigorously stirred at room temperature for 1.5 h. The reaction mixture was diluted with DCM and saturated aqueous NaHCO₃. The layers were separated and the aqueous layer was extracted again with DCM. The combined organic layer was washed with water, dried on MgSO₄, filtered, and evaporated. The residue was purified by column chromatography on silica gel (Biotage Sfar 50 g; eluent: (AcOEt/EtOH (3:1))/n-heptane, gradient from 0:100 to 40:60) to give Intermediate 70 (1243 mg, yield: 49%) as an off-white solid.

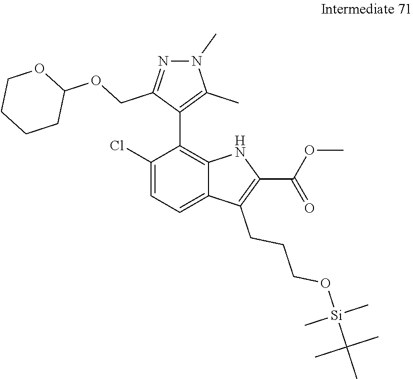 [SMILES: COC(=O)c1[nH]c2c(-c3c(COC4CCCCO4)nn(C)c3C)c(Cl)ccc2c1CCCO[Si](C)(C)C(C)(C)C]

3,4-Dihydro-2H-pyran (336 μL, 3.684 mmol, 1.5 eq.) and PTSA·H₂O (47 mg, 0.246 mmol, 0.1 eq.) were added to a solution of Intermediate 70 (1243 mg, 2.456 mmol) in dry DCM (50 mL) at room temperature. The reaction mixture was stirred overnight at room temperature. The reaction mixture was diluted with DCM and was washed with saturated aqueous NaHCO₃. The organic layer was dried on MgSO₄, filtered, and evaporated. The residue was purified by column chromatography on silica gel (Biotage SNAP Ultra 50 g; eluent: (AcOEt/EtOH (3:1))/n-heptane, gradient from 0:100 to 40:60) to give Intermediate 71 (1627 mg, quantitative) as a thick light brown oil.

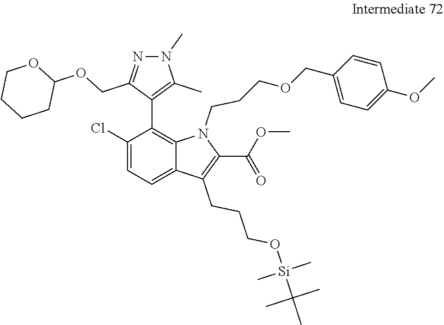 [SMILES: COC(=O)c1c(CCCO[Si](C)(C)C(C)(C)C)c2ccc(Cl)c(-c3c(COC4CCCCO4)nn(C)c3C)c2n1CCCOCc1ccc(OC)cc1]

Cesium carbonate (1208 mg, 3.705 mmol, 1.5 eq.) was added to a solution of Intermediate 71 (90% pure, 1620 mg, 2.47 mmol) and 1-[(3-iodopropoxy)methyl]-4-methoxybenzene (CAS [198411-17-5], 1134 mg, 3.705 mmol, 1.5 eq.) in dry DMF (30 mL) at room temperature. The reaction mixture was stirred overnight at 60° C. The mixture was diluted with EtOAc and water. The layers were separated and the aqueous layer was extracted again twice with EtOAc. The combined organic layer was dried on MgSO₄, filtered, and the solvent was evaporated. The residue was purified by column chromatography on silica gel (Biotage Sfar 25 g; eluent: (AcOEt/EtOH (3:1))/n-heptane, gradient from 0:100 to 40:60) to give Intermediate 72 (1802 mg, yield: 89%) as a brown oil.

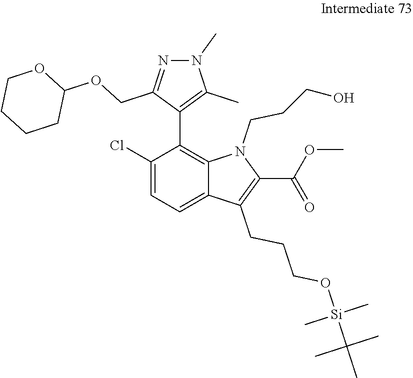 [SMILES: COC(=O)c1c(CCCO[Si](C)(C)C(C)(C)C)c2ccc(Cl)c(-c3c(COC4CCCCO4)nn(C)c3C)c2n1CCCO]

DDQ (1001 mg, 4.408 mmol, 2 eq.) was added to a solution of Intermediate 72 (1802 mg, 2.204 mmol) in DCM (100 mL) and water (10 mL). The reaction mixture was vigorously stirred at room temperature for 3 h. The reaction mixture was diluted with DCM and saturated aqueous NaHCO₃. The layers were separated and the aqueous layer was extracted again with DCM. The combined organic layer was dried on MgSO₄, filtered, and evaporated. The residue was purified by column chromatography on silica gel (Biotage Sfar 50 g; eluent: (AcOEt/EtOH (3:1))/n-heptane, gradient from 0:100 to 40:60) to give Intermediate 73 (1200 mg, yield: 84%) as a brown oil.

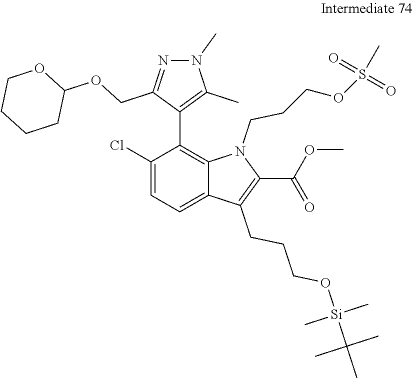 [SMILES: COC(=O)c1c(CCCO[Si](C)(C)C(C)(C)C)c2ccc(Cl)c(-c3c(COC4CCCCO4)nn(C)c3C)c2n1CCCOS(C)(=O)=O]

DIPEA (532 μL, 3.085 mmol, 2 eq.) and methanesulfonic anhydride (591 mg, 3.393 mmol, 2.2 eq.) were added to a solution of Intermediate 73 (1 g, 1.542 mmol) in DCM (10 mL) and the reaction mixture was stirred for 4 h at room temperature. Water was added and the layers were separated. The aqueous layer was extracted with DCM. The combined organic layer was dried with MgSO₄, filtered, and evaporated to give Intermediate 74 (1.2 g, 90% pure, yield: 96%) used without further purification.

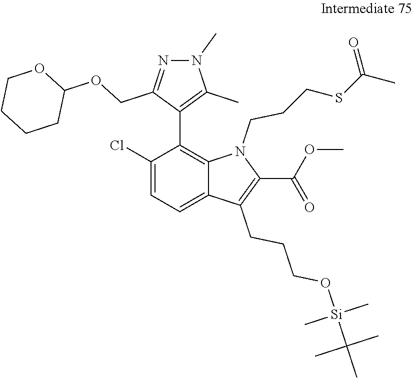 [SMILES: COC(=O)c1c(CCCO[Si](C)(C)C(C)(C)C)c2ccc(Cl)c(-c3c(COC4CCCCO4)nn(C)c3C)c2n1CCCSC(C)=O]

Intermediate 74 (1.2 g, 1.652 mmol) was dissolved in dry ACN (13 mL) and potassium thioacetate (566 mg, 4.956 mmol, 3 eq.) was added. The reaction mixture was then stirred at 60° C. for 16 h. The solvent was evaporated and the residue was partitioned between water and EtOAc. The layers were separated and the aqueous layer was extracted again with EtOAc. The combined organic layer was dried with MgSO₄, filtered, and evaporated. The residue was purified by column chromatography on silica gel (SNAP Ultra 25 g; eluent: (AcOEt/EtOH (3:1))/n-heptane, gradient from 0:100 to 40:60) to give Intermediate 75 (1.2 g, 87% pure, yield: 89%) as a light yellow oil.

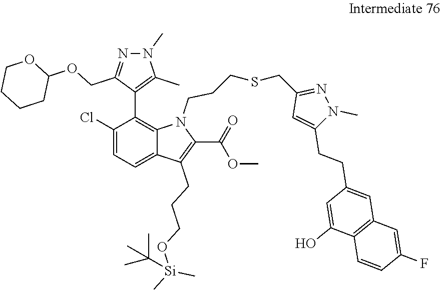 [SMILES: COC(=O)c1c(CCCO[Si](C)(C)C(C)(C)C)c2ccc(Cl)c(-c3c(COC4CCCCO4)nn(C)c3C)c2n1CCCSCc1cc(CCc2cc(O)c3ccc(F)cc3c2)n(C)n1]

Intermediate 75 (1.2 g, 1.478 mmol) was dissolved in MeOH (12 mL) and this solution was degassed with nitrogen for 5 min (solution A). A suspension of Intermediate 7 (1054 mg, 1.626 mmol, 1.1 eq.) and K₂CO₃ (613 mg, 4.434 mmol, 3 eq.) in dry MeOH (12 mL) and THF (7 mL) was degassed with nitrogen for 5 min (solution B). Solution A was then added dropwise to solution B at room temperature under nitrogen atmosphere. The reaction mixture was stirred at room temperature under nitrogen atmosphere for 2 h. The solvents were evaporated and the residue was purified by column chromatography on silica gel (SNAP Ultra 25 g; eluent: (AcOEt/EtOH (3:1))/n-heptane, gradient from 0:100 to 40:60) to give Intermediate 76 (1010 mg, yield: 72%).

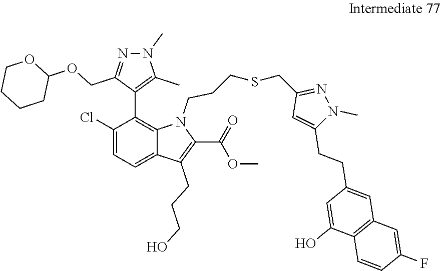 [SMILES: COC(=O)c1c(CCCO)c2ccc(Cl)c(-c3c(COC4CCCCO4)nn(C)c3C)c2n1CCCSCc1cc(CCc2cc(O)c3ccc(F)cc3c2)n(C)n1]

TBAF (1 M in TIE, 1.174 mL, 1.174 mmol, 1.1 eq.) was added to a solution of Intermediate 76 (1.01 g, 1.067 mmol) in dry THF (9 mL) and the reaction mixture was stirred at room temperature for 16 h. The reaction was quenched by addition of water and the mixture was extracted with EtOAc. The organic layer was dried with MgSO₄, filtered, and evaporated to give Intermediate 77 (800 mg, 90% pure, yield: 81%), used without further purification.

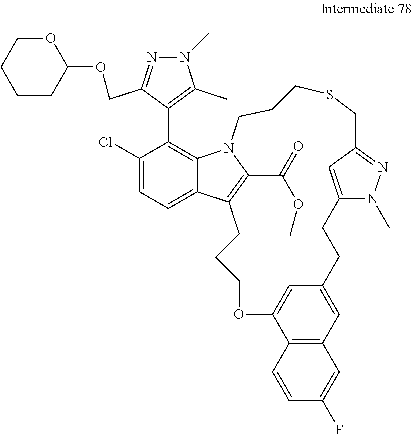 [SMILES: COC(=O)c1c2c3ccc(Cl)c(-c4c(COC5CCCCO5)nn(C)c4C)c3n1CCCSCc1cc(n(C)n1)CCc1cc(c3ccc(F)cc3c1)OCCC2]

Intermediate 78 was prepared according to an analogous procedure as for Intermediate 43, using Intermediate 77 instead of Intermediate 42.

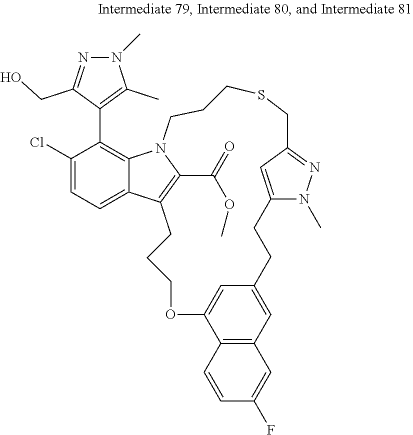 [SMILES: COC(=O)c1c2c3ccc(Cl)c(-c4c(CO)nn(C)c4C)c3n1CCCSCc1cc(n(C)n1)CCc1cc(c3ccc(F)cc3c1)OCCC2]

### Intermediate 79: Mixture of Stereoisomers

### Intermediate 80: Sₐ or Rₐ; One Atropisomer but Absolute Stereochemistry Undetermined

### Intermediate 81: Rₐ or Sₐ; One Atropisomer but Absolute Stereochemistry Undetermined

pTsOH (230 mg, 1.338 mmol, 2 eq.) was added to a solution of Intermediate 78 (681 mg, 0.669 mmol) in MeOH (3 mL) and the reaction mixture was stirred at room temperature for 16 h. The solvent was evaporated and the residue was taken up in EtOAc and the solution was washed with water. The organic layer was dried with MgSO₄, filtered, and evaporated to give Intermediate 79 (480 mg, 80% pure, yield: 78%). A fraction of Intermediate 79 (350 mg) was purified by preparative SFC (Stationary phase: Chiralpak Daicel ID 20×250 mm, Mobile phase: CO₂, EtOH+0.4% iPrNH₂) to give Intermediate 80 (43 mg, yield: 9%) and Intermediate 81 (43 mg, yield: 9%).

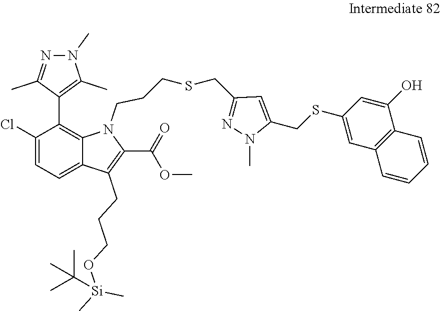 [SMILES: COC(=O)c1c(CCCO[Si](C)(C)C(C)(C)C)c2ccc(Cl)c(-c3c(C)nn(C)c3C)c2n1CCCSCc1cc(CSc2cc(O)c3ccccc3c2)n(C)n1]

Intermediate 13 (712 mg, 1.022 mmol) was dissolved in dry MeOH (15 mL) and this solution was degassed by bubbling nitrogen through for 5 min (solution A). A suspension of Intermediate 46 (702 mg, 1.124 mmol, 1.1 eq.) and K₂CO₃ (282 mg, 2.043 mmol, 2 eq.) in dry MeOH (15 mL) and THF (15 mL) was degassed by bubbling nitrogen through for 5 min (solution B). Solution A was then added to solution B at room temperature under nitrogen atmosphere. The reaction mixture was stirred overnight at room temperature under nitrogen atmosphere. The solvents were evaporated and the residue was partitioned between water and EtOAc. The layers were separated and the aqueous layer was extracted again with EtOAc. The combined organic layer was dried on MgSO₄, filtered, and evaporated to give Intermediate 82 (923 mg, ˜1:1 mixture with the lactone corresponding to Intermediate 13) as a brown solid, used without further purification.

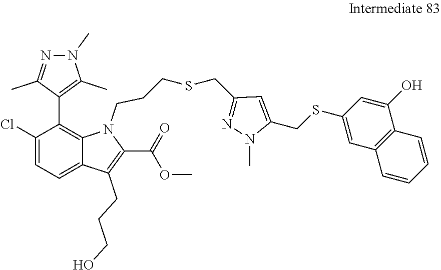 [SMILES: COC(=O)c1c(CCCO)c2ccc(Cl)c(-c3c(C)nn(C)c3C)c2n1CCCSCc1cc(CSc2cc(O)c3ccccc3c2)n(C)n1]

Intermediate 83 was prepared according to an analogous procedure as for Intermediate 42, using Intermediate 82 instead of Intermediate 41.

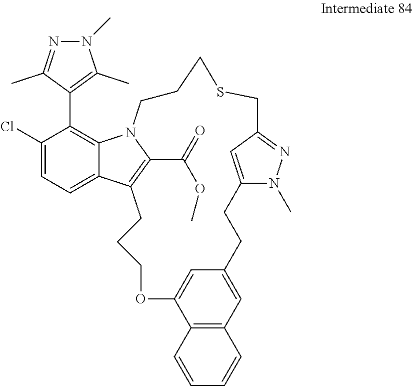

Intermediate 84 was prepared according to an analogous procedure as for Intermediate 43, using Intermediate 83 instead of Intermediate 42.

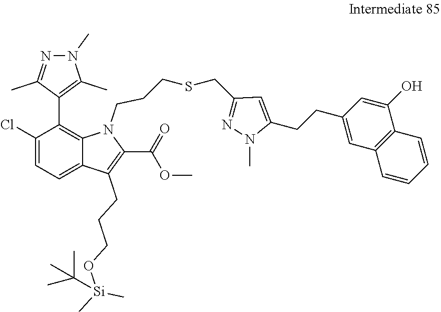 [SMILES: COC(=O)c1c(CCCO[Si](C)(C)C(C)(C)C)c2ccc(Cl)c(-c3c(C)nn(C)c3C)c2n1CCCSCc1cc(CCc2cc(O)c3ccccc3c2)n(C)n1]

Intermediate 85 was prepared according to an analogous procedure as for Intermediate 82, using Intermediate 40 instead of Intermediate 46.

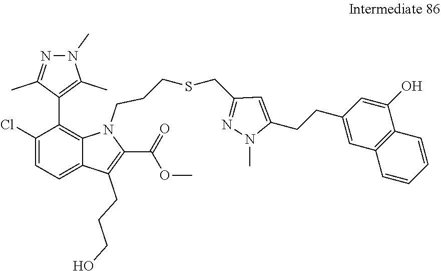 [SMILES: COC(=O)c1c(CCCO)c2ccc(Cl)c(-c3c(C)nn(C)c3C)c2n1CCCSCc1cc(CCc2cc(O)c3ccccc3c2)n(C)n1]

Intermediate 86 was prepared according to an analogous procedure as for Intermediate 42, using Intermediate 85 instead of Intermediate 41.

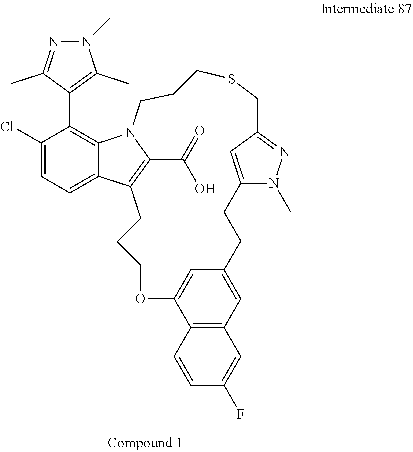

A solution of Intermediate 16 (12 mg, 0.017 mmol) and LiOH (4 mg, 0.168 mmol, 10 eq.) in THF (1 mL), water (1 mL) and MeOH (1 mL) was stirred at 50° C. overnight. The reaction mixture was concentrated to the aqueous layer. This aqueous solution was diluted with water and was washed with a small amount of EtOAc. Aqueous HCl (1 N) was added dropwise to the aqueous layer until the solution became cloudy. The mixture was extracted twice with EtOAc. The combined organic layer was dried by filtration on Extrelut NT3, and evaporated. As some starting material was still present, the residue was dissolved in THF (1 mL), MeOH (1 mL), and water (1 mL). LiOH (4 mg, 0.168 mmol, 10 eq.) was added and the reaction mixture was stirred at 60° C. for 4 h. The reaction mixture was concentrated to the aqueous layer. This aqueous solution was diluted with water. Aqueous HCl (1 N) was added dropwise to the aqueous layer until the solution became cloudy. The mixture was extracted twice with EtOAc. The combined organic layer was dried by filtration on Extrelut NT3, and evaporated to give Compound 1 (11 mg, yield: 94%) as a light brown solid, dried under vacuum at 50° C.

¹H NMR (400 MHz, CHLOROFORM-d) δ ppm 1.71-1.90 (m, 3H) 1.93 (s, 3H) 1.95 (s, 3H) 2.39 (br t, J=5.7 Hz, 2H) 2.85-3.01 (m, 4H) 3.32 (s, 6H) 3.34-3.49 (m, 2H) 3.79 (s, 4H) 3.83-3.99 (m, 3H) 5.65 (s, 1H) 6.02 (s, 1H) 7.01-7.07 (m, 2H) 7.11 (d, J=8.6 Hz, 1H) 7.22-7.26 (m, 1H) 7.59 (d, J=8.5 Hz, 1H) 7.76 (br dd, J=8.9, 6.2 Hz, 1H)

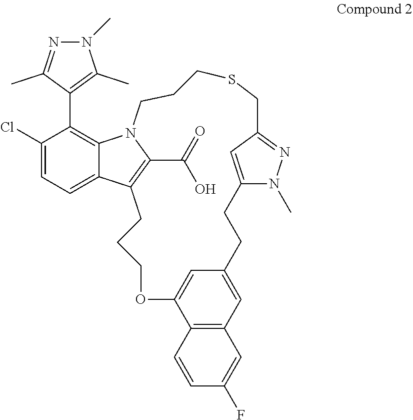

### Compound 2: Rₐ or Sₐ; One Atropisomer but Absolute Stereochemistry Undetermined

A solution of Intermediate 17 (46 mg, 0.0644 mmol) and LiOH (16 mg, 0.644 mmol, 10 eq.) in THE (3 mL), water (3 mL) and MeOH (3 mL) was stirred at 60° C. overnight. The reaction mixture was concentrated to the aqueous layer. Aqueous HCl (1 N) was added dropwise to the aqueous layer until the solution became cloudy. The mixture was extracted twice with EtOAc. The combined organic layer was dried by filtration on Extrelut NT3, and evaporated. The residue was purified by column chromatography (Biotage Sfar 10 g; eluent: DCM/10% MeOH in DCM 100/0 to 0/100) to give Compound 2 (38 mg, yield: 84%) as a white solid.

¹H NMR (400 MHz, CHLOROFORM-d) δ ppm 1.14-1.31 (m, 3H), 1.78-1.93 (m, 2H), 1.98 (s, 3H), 2.01 (s, 3H), 2.34-2.42 (m, 4H), 2.84-3.01 (m, 5H), 3.20 (s, 3H), 3.29-3.43 (m, 4H), 3.81 (s, 3H), 3.89 (tdd, J=15.4, 15.4, 9.6, 5.6 Hz, 2H), 4.14 (br d, J=38.3 Hz, 2H), 5.66 (s, 1H), 6.00 (s, 1H), 7.07 (d, J=8.8 Hz, 1H), 7.08-7.14 (m, 2H), 7.27-7.32 (m, 1H), 7.57 (d, J=8.6 Hz, 1H), 7.99 (dd, J=9.1, 5.8 Hz, 1H).

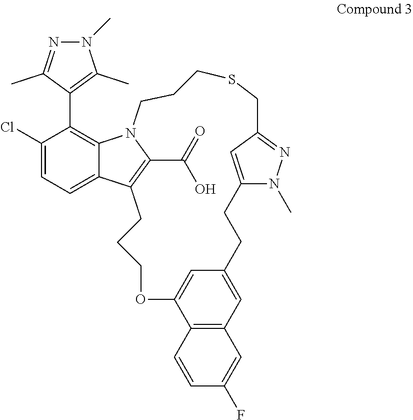

### Compound 3: Sₐ or Rₐ; One Atropisomer but Absolute Stereochemistry Undetermined

Compound 3 was prepared according to an analogous procedure as for Compound 2, starting from Intermediate 18 instead of Intermediate 17.

¹H NMR (400 MHz, CHLOROFORM-d) δ ppm 1.12-1.31 (m, 3H), 1.78-1.91 (m, 2H), 1.98 (s, 3H), 2.00 (s, 3H), 2.33-2.41 (m, 4H), 2.84-3.03 (m, 4H), 3.21 (s, 3H), 3.28-3.42 (m, 4H), 3.80-3.82 (m, 3H), 3.82-3.98 (m, 2H), 4.00-4.30 (m, 2H), 5.66 (s, 1H), 6.00 (s, 1H), 7.05-7.13 (m, 3H), 7.27-7.30 (m, 1H), 7.57 (d, J=8.6 Hz, 1H), 7.97 (br d, J=3.3 Hz, 1H).

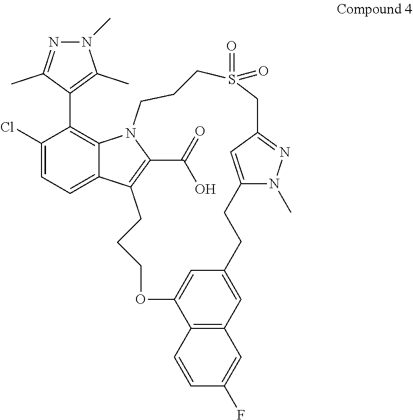

### Compound 4: Rₐ or Sₐ; One Atropisomer but Absolute Stereochemistry Undetermined

A solution of Intermediate 19 (24 mg, 0.0322 mmol) and LiOH (8 mg, 0.322 mmol, 10 eq.) in THE (2 mL), water (2 mL) and MeOH (2 mL) was stirred at 60° C. overnight. The reaction mixture was concentrated to the aqueous layer. Aqueous HCl (1 N) was added dropwise to the aqueous layer until the solution became cloudy. The gel-like mixture was extracted twice with EtOAc. The combined organic layer was dried by filtration on Extrelut NT3 (Supelco®), and evaporated to give Compound 4 (21 mg, yield: 89%) as an off-white solid.

¹H NMR (400 MHz, CHLOROFORM-d) δ ppm 1.57-1.71 (m, 3H) 1.92-2.15 (m, 9H) 2.22-2.45 (m, 5H) 2.74 (s, 4H) 2.93 (br s, 6H) 3.43 (br s, 4H) 3.53-3.72 (m, 3H) 3.84 (s, 4H) 3.92 (s, 2H) 5.83 (s, 1H) 6.00 (s, 1H) 6.89 (d, J=8.6 Hz, 1H) 7.17 (s, 1H) 7.22 (td, J=8.8, 2.4 Hz, 1H) 7.31-7.38 (m, 2H) 7.60 (d, J=8.6 Hz, 1H) 8.22 (br s, 1H).

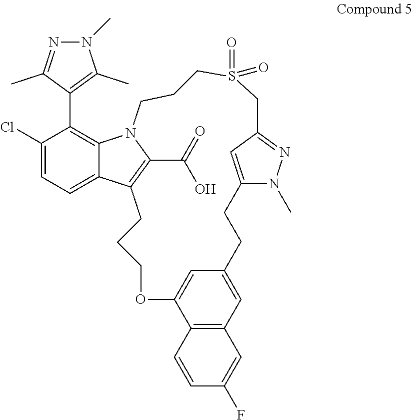

### Compound 5: Sₐ or Rₐ; One Atropisomer but Absolute Stereochemistry Undetermined

Compound 5 was prepared according to an analogous procedure as for Compound 4, starting from Intermediate 20 instead of Intermediate 19.

¹H NMR (400 MHz, CHLOROFORM-d) δ ppm 1.47-1.72 (m, 4H) 1.88-2.15 (m, 8H) 2.20-2.45 (m, 5H) 2.76 (s, 3H) 2.94 (br s, 4H) 3.38-3.78 (m, 6H) 3.80-3.96 (m, 7H) 5.84 (s, 1H) 5.99 (s, 1H) 6.90 (d, J=8.6 Hz, 1H) 7.16 (s, 1H) 7.22 (td, J=8.7, 2.4 Hz, 1H) 7.34 (dd, J=10.1, 2.4 Hz, 2H) 7.60 (d, J=8.6 Hz, 1H) 8.14-8.25 (m, 2H).

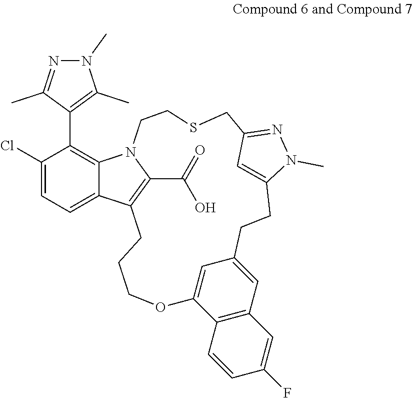

### Compound 6: Rₐ or Sₐ; One Atropisomer but Absolute Stereochemistry Undetermined

### Compound 7: Sₐ or Rₐ; One Atropisomer but Absolute Stereochemistry Undetermined

A solution of LiOH (26 mg, 1.071 mmol, 15 eq.) in water (0.5 mL) was added to a solution of Intermediate 26 (50 mg, 0.0714 mmol) in THF (0.7 mL) and MeOH (0.7 mL). The reaction mixture was stirred at 60° C. for 6 h, then at room temperature for 16 h and, finally, at 70° C. for 4 h. The mixture was cooled to room temperature, diluted with water (5 mL), and treated with aqueous HCl (1 N) until acidic pH. The aqueous layer was extracted with DCM (3×15 mL) and then with a DCM/MeOH (9/1) mixture (3×10 mL). The combined organic layer was dried over MgSO₄, filtered, and evaporated. The residue was purified by preparative SFC (Stationary phase: Chiralcel Diacel IH 20×250 mm, Mobile phase: CO₂, EtOH+0.4% iPrNH₂) to give two fractions. Both fractions were suspended in water (3 mL) and treated with aqueous HCl (1 N, a few drops). The aqueous layers were then extracted with a mixture of DCM/MeOH (9/1, 3×10 mL). The combined organic layers were dried over MgSO₄, filtered, and evaporated to give Compound 6 (12 mg, yield: 25%) and Compound 7 (15 mg, yield: 31%), both as white solids.

Compound 6: ¹H NMR (80° C., 400 MHz, DMSO-d₆) δ ppm 1.64-1.81 (m, 5H), 1.88 (br s, 3H), 2.29-2.38 (m, 2H), 2.91-3.10 (m, 6H), 3.19-3.39 (m, 2H), 3.52 (s, 3H), 3.70 (s, 3H), 4.08 (t, J=6.1 Hz, 2H), 5.36 (s, 1H), 6.32 (s, 1H), 7.01 (td, J=8.9, 2.7 Hz, 1H), 7.07 (s, 1H), 7.18 (d, J=8.5 Hz, 1H), 7.35 (dd, J=10.6, 2.6 Hz, 1H), 7.64 (br dd, J=9.2, 5.9 Hz, 1H), 7.72 (d, J=8.5 Hz, 1H).

Compound 7: ¹H NMR (100° C., 400 MHz, DMSO-d₆) δ ppm 1.70-1.83 (m, 5H), 1.89 (s, 3H), 2.30-2.38 (m, 2H), 2.86-3.02 (m, 4H), 3.09 (s, 2H), 3.20-3.38 (m, 2H), 3.50 (s, 3H), 3.70 (s, 3H), 4.08 (t, J=6.2 Hz, 2H), 5.39 (s, 1H), 6.31 (s, 1H), 7.02 (td, J=8.9, 2.6 Hz, 1H), 7.08 (s, 1H), 7.17 (d, J=8.5 Hz, 1H), 7.34 (dd, J=10.5, 2.6 Hz, 1H), 7.66-7.73 (m, 2H).

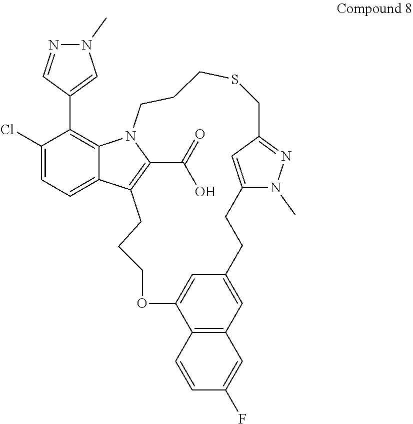 [SMILES: Cn1cc(-c2c(Cl)ccc3c4c(C(=O)O)n(c23)CCCSCc2cc(n(C)n2)CCc2cc(c3ccc(F)cc3c2)OCCC4)cn1]

A solution of Intermediate 33 (28 mg, 0.0408 mmol) and LiOH (10 mg, 0.408 mmol, 10 eq.) in THE (2 mL), water (2 mL) and MeOH (2 mL) was stirred at 60° C. overnight. The reaction mixture was concentrated to the aqueous layer and aqueous HCl (1 N) was added dropwise until the solution became cloudy. The gel-like mixture was extracted twice with EtOAc. The combined organic layer was dried by filtration on Extrelut NT3, and evaporated. The residue was purified by column chromatography (Biotage Sfar 10 g; eluent: DCM/10% MeOH in DCM, from 100/0 to 0/100) to afford Compound 8 (24 mg, yield: 87%) as a white solid.

¹H NMR (400 MHz, CHLOROFORM-d) δ ppm 0.75-0.90 (m, 3H) 1.20-1.34 (m, 4H) 1.73 (br t, J=6.9 Hz, 2H) 2.33-2.43 (m, 2H) 2.88-3.01 (m, 4H) 3.31-3.42 (m, 7H) 3.88-4.06 (m, 7H) 5.67 (s, 1H) 6.07 (s, 1H) 6.96-7.04 (m, 2H) 7.12 (d, J=8.6 Hz, 1H) 7.23 (dd, J=10.1, 2.4 Hz, 1H) 7.30 (s, 1H) 7.38 (s, 1H) 7.47 (br d, J=8.1 Hz, 1H) 7.61 (d, J=8.6 Hz, 1H).

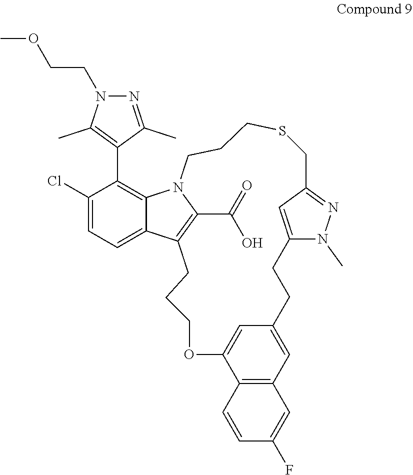

### Compound 9: Sₐ or Rₐ; One Atropisomer but Absolute Stereochemistry Undetermined

Compound 9 was prepared according to an analogous procedure as for Compound 8, starting from Intermediate 55 instead of Intermediate 33.

¹H NMR (400 MHz, CHLOROFORM-d, 27° C.) 6 ppm 1.19-1.31 (m, 3H) 1.81-1.95 (m, 2H) 1.99 (s, 3H) 2.05 (s, 3H) 2.39 (br t, J=5.9 Hz, 2H) 2.83-3.01 (m, 4H) 3.18 (s, 3H) 3.30 (s, 3H) 3.31 (d, J=2.0 Hz, 2H) 3.36 (br d, J=6.4 Hz, 2H) 3.67-3.74 (m, 1H) 3.78 (dt, J=10.0, 5.9 Hz, 1H) 3.82-3.88 (m, 1H) 3.89-3.96 (m, 1H) 4.04-4.16 (m, 1H) 4.23 (t, J=5.3 Hz, 2H) 5.63 (s, 1H) 6.00 (s, 1H) 7.05 (d, J=8.6 Hz, 1H) 7.09 (s, 1H) 7.11 (br dd, J=9.0, 2.0 Hz, 1H) 7.28 (dd, J=10.1, 2.4 Hz, 1H) 7.56 (d, J=8.6 Hz, 1H) 8.01 (dd, J=9.1, 5.8 Hz, 1H).

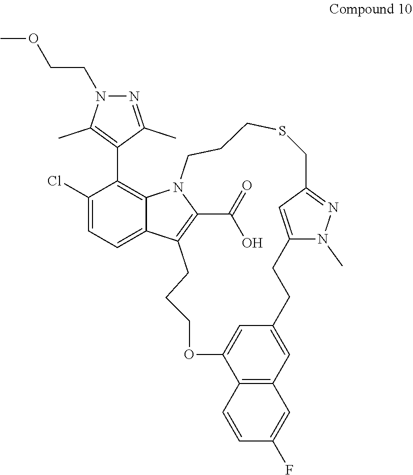

### Compound 10: Rₐ or Sₐ; One Atropisomer but Absolute Stereochemistry Undetermined

Compound 10 was prepared according to an analogous procedure as for Compound 8, starting from Intermediate 56 instead of Intermediate 33.

¹H NMR (400 MHz, CHLOROFORM-d, 27° C.) 6 ppm 1.12-1.31 (m, 3H) 1.89 (q, J=6.7 Hz, 2H) 1.98 (s, 3H) 2.03 (s, 3H) 2.33-2.45 (m, 2H) 2.95 (br s, 4H) 3.21 (s, 3H) 3.29 (s, 3H) 3.30-3.32 (m, 2H) 3.36 (br s, 2H) 3.66-3.73 (m, 1H) 3.74-3.81 (m, 1H) 3.82-3.89 (m, 1H) 3.89-3.97 (m, 1H) 4.08 (br s, 1H) 4.22 (t, J=5.4 Hz, 1H) 5.64 (s, 1H) 6.00 (s, 1H) 7.06 (d, J=8.6 Hz, 1H) 7.08 (s, 1H) 7.09 (br dd, J=9.0, 2.6 Hz, 1H) 7.27 (dd, J=9.9, 2.4 Hz, 2H) 7.56 (d, J=8.6 Hz, 1H) 7.96 (dd, J=9.1, 5.8 Hz, 1H).

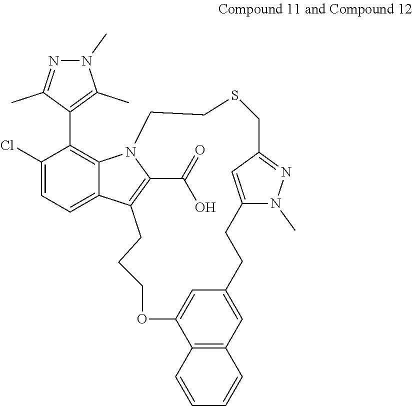

### Compound 11: Rₐ or Sₐ; One Atropisomer but Absolute Stereochemistry Undetermined

### Compound 12: Sₐ or Rₐ; One Atropisomer but Absolute Stereochemistry Undetermined

A solution of Intermediate 43 (200 mg, 0.293 mmol) and LiOH (70 mg, 2.931 mmol, 10 eq.) in THE (7 mL), water (7 mL), and MeOH (7 mL) was stirred at 60° C. overnight. The reaction mixture was concentrated to the aqueous layer. Aqueous HCl (1 N) was added dropwise to the aqueous layer until the solution became cloudy. The mixture was extracted twice with EtOAc. The combined organic layer was dried on MgSO₄, filtered, and evaporated. The residue was purified by preparative SFC (Stationary phase: Chiralpak Daicel IG 20×250 mm, Mobile phase: CO₂, EtOH+0.4% iPrNH₂) followed by preparative HPLC (Stationary phase: RP XBridge Prep C18 OBD-10 μm, 50×150 mm, Mobile phase: 0.25% NH₄HCO₃ solution in water, CH₃CN) to give Compound 11 (4 mg, yield: 2%) as a colourless film and Compound 12 (57 mg, yield: 29%) as a white solid.

Compound 11

¹H NMR (400 MHz, CHLOROFORM-d) δ ppm 1.76-2.03 (m, 7H), 2.35 (br s, 2H), 2.78-3.05 (m, 6H), 3.15 (br s, 6H), 3.43 (br s, 5H), 3.81 (s, 4H), 3.98 (br s, 2H), 4.64-4.89 (m, 1H), 5.51 (br s, 1H), 6.03 (s, 1H), 7.13-7.20 (m, 2H), 7.31 (br t, J=7.3 Hz, 1H), 7.39 (t, J=7.6 Hz, 1H), 7.59 (d, J=8.6 Hz, 1H), 7.67 (d, J=8.2 Hz, 1H), 7.85-7.99 (m, 1H).

Compound 12

¹H NMR (400 MHz, CHLOROFORM-d) δ ppm 1.71-2.09 (m, 8H), 2.35 (br s, 2H), 2.85-3.07 (m, 5H), 3.07-3.34 (m, 4H), 3.44 (br s, 4H), 3.75-3.86 (m, 4H), 3.86-4.09 (m, 3H), 4.39 (br s, 3H), 4.74 (br s, 1H), 5.47 (br s, 1H), 6.02 (s, 1H), 7.13 (d, J=8.6 Hz, 1H), 7.16 (s, 1H), 7.27-7.35 (m, 1H), 7.39 (t, J=7.2 Hz, 1H), 7.58 (d, J=8.6 Hz, 1H), 7.66 (d, J=8.0 Hz, 1H), 7.93 (br s, 1H).

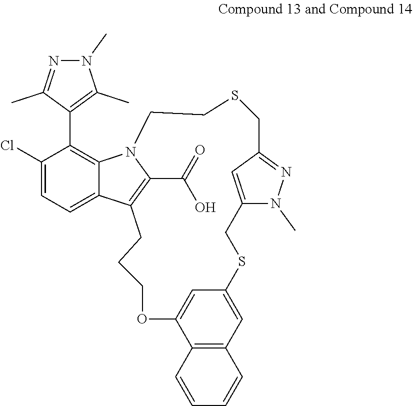

### Compound 13: Rₐ or Sₐ; One Atropisomer but Absolute Stereochemistry Undetermined

### Compound 14: Sₐ or Rₐ; One Atropisomer but Absolute Stereochemistry Undetermined

A solution of Intermediate 59 (165 mg, 0.236 mmol) and LiOH (56 mg, 2.356 mmol, 10 eq.) in THF (7 mL), water (7 mL), and MeOH (7 mL) was stirred at 60° C. overnight. The reaction mixture was concentrated to the aqueous layer. Aqueous HCl (1 N) was added dropwise to the aqueous layer until the solution became cloudy. The mixture was extracted twice with EtOAc. The combined organic layer was dried on MgSO₄, filtered, and evaporated. The residue was purified by preparative SFC (Stationary phase: Chiralpak Daicel IH 20×250 mm, Mobile phase: CO₂, EtOH+0.4% iPrNH₂) followed by preparative HPLC (Stationary phase: RP XBridge Prep C18 OBD-10 μm, 50×150 mm, Mobile phase: 0.25% NH₄HCO₃ solution in water, CH₃CN) to give Compound 13 (44 mg, yield: 27%) as a white solid, and impure Compound 14 (contaminated by iPrNH₂). This impure batch was suspended in water and a few drops of 1 N aqueous HCl were added. The mixture was extracted by EtOAc followed by DCM. The combined organic layer was dried by filtration on Extrelut NT3, and evaporated to give Compound 14 (24 mg, yield: 15%) as an off-white solid.

Compound 13

¹H NMR (400 MHz, CHLOROFORM-d) δ ppm 1.49 (br s, 2H), 1.94 (br s, 6H), 2.41 (br d, J=5.1 Hz, 2H), 2.90-3.56 (m, 9H), 3.79 (d, J=3.9 Hz, 6H), 3.92 (s, 2H), 3.98-4.08 (m, 2H), 5.38 (s, 1H), 6.26-6.30 (m, 1H), 7.11 (d, J=8.6 Hz, 1H), 7.29 (br d, J=7.7 Hz, 1H), 7.36-7.43 (m, 1H), 7.45 (s, 1H), 7.59 (d, J=8.5 Hz, 1H), 7.65 (br d, J=8.2 Hz, 2H).

Compound 14

¹H NMR (400 MHz, CHLOROFORM-d) δ ppm 1.29-1.56 (m, 2H), 1.93 (br s, 5H), 2.40 (br s, 2H), 2.91-3.64 (m, 8H), 3.79 (s, 6H), 3.88-3.97 (m, 2H), 3.98-4.08 (m, 2H), 5.39 (s, 1H), 6.28 (d, J=1.3 Hz, 1H), 7.12 (d, J=8.5 Hz, 1H), 7.29 (s, 1H), 7.39 (t, J=7.1 Hz, 1H), 7.44 (s, 1H), 7.60 (d, J=8.6 Hz, 2H), 7.64 (d, J=8.3 Hz, 2H).

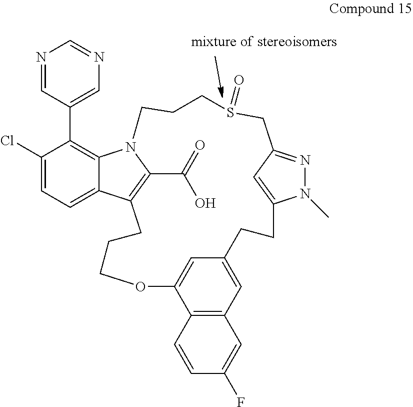 [SMILES: Cn1nc2cc1CCc1cc(c3ccc(F)cc3c1)OCCCc1c(C(=O)O)n(c3c(-c4cncnc4)c(Cl)ccc13)CCCS(=O)C2]

Compound 15 was prepared according to an analogous procedure as for Compound 4, starting from Intermediate 68 instead of Intermediate 19.

¹H NMR (400 MHz, DMSO-d₆) δ ppm 1.74-1.88 (m, 2H) 2.07-2.26 (m, 2H) 2.26-2.38 (m, 3H) 2.89-3.06 (m, 9H) 3.19 (s, 11H) 3.22-3.34 (m, 6H) 3.57-3.68 (m, 3H) 3.82 (br s, 2H) 3.86-3.96 (m, 2H) 3.96-3.96 (m, 1H) 4.10-4.26 (m, 1H) 5.74 (s, 1H) 6.17 (s, 1H) 7.02 (d, J=8.6 Hz, 1H) 7.20-7.29 (m, 2H) 7.51 (dd, J=10.5, 2.5 Hz, 1H) 7.74 (d, J=8.6 Hz, 1H) 8.04 (Dd, J=9.0, 5.9 Hz, 1H) 8.82 (Br s, 1H) 8.96 (Br s, 1H) 9.31 (s, 1H).

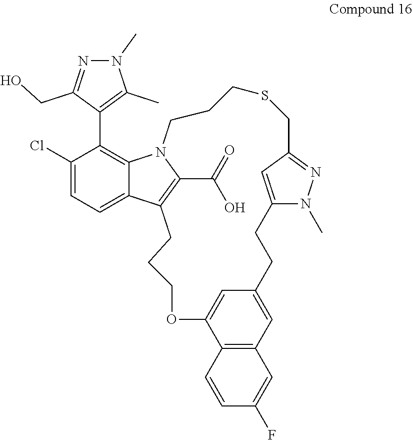 [SMILES: Cc1c(-c2c(Cl)ccc3c4c(C(=O)O)n(c23)CCCSCc2cc(n(C)n2)CCc2cc(c3ccc(F)cc3c2)OCCC4)c(CO)nn1C]

Compound 16: Sₐ or Rₐ; one atropisomer but absolute stereochemistry undetermined LiOH (14 mg, 0.589 mmol, 10 eq.) was added to a solution of Intermediate 80 (43 mg, 0.059 mmol) in THF (2 mL), MeOH (2 mL), and water (1 mL) and the reaction mixture was stirred at 55° C. for 16 h. The solvents were evaporated and the residue was purified by column chromatography on silica gel (DCM/MeOH from 100/0 to 90/10) to give Compound 16 (14 mg, yield: 33%).

¹H NMR (400 MHz, CHLOROFORM-d, 27° C.) 6 ppm 1.16-1.38 (m, 2H) 1.59-1.71 (m, 1H) 1.75-1.85 (m, 1H) 2.02 (s, 3H) 2.26-2.46 (m, 2H) 2.85-3.02 (m, 7H) 3.21-3.31 (m, 2H) 3.34-3.47 (m, 2H) 3.70-3.80 (m, 1H) 3.82-3.90 (m, 1H) 3.85 (s, 3H) 4.05 (br s, 1H) 4.19 (br s, 1H) 4.41 (s, 2H) 5.73 (s, 1H) 6.00 (s, 1H) 7.03 (d, J=8.6 Hz, 1H) 7.09-7.16 (m, 2H) 7.32 (dd, J=10.0, 2.5 Hz, 1H) 7.58 (d, J=8.6 Hz, 1H) 8.08 (dd, J=9.1, 5.8 Hz, 1H).

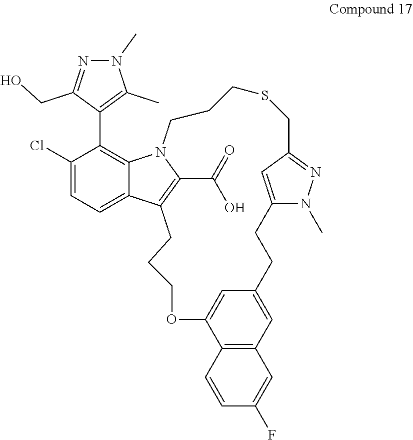 [SMILES: Cc1c(-c2c(Cl)ccc3c4c(C(=O)O)n(c23)CCCSCc2cc(n(C)n2)CCc2cc(c3ccc(F)cc3c2)OCCC4)c(CO)nn1C]

### Compound 17: Rₐ or Sₐ; One Atropisomer but Absolute Stereochemistry Undetermined

Compound 17 was prepared according to an analogous procedure as for Compound 16, starting from Intermediate 81 instead of Intermediate 80.

¹H NMR (400 MHz, CHLOROFORM-d, 27° C.) δ ppm 1.14-1.36 (m, 2H) 1.59-1.69 (m, 1H) 1.76-1.84 (m, 1H) 2.01 (s, 3H) 2.27-2.46 (m, 2H) 2.82-3.09 (m, 4H) 3.04 (s, 3H) 3.20-3.31 (m, 2H) 3.37-3.45 (m, 2H) 3.72-3.80 (m, 1H) 3.81-3.91 (m, 1H) 3.84 (s, 3H) 4.03 (br s, 1H) 4.20 (br s, 1H) 4.39 (br s, 2H) 5.72 (s, 1H) 6.00 (s, 1H) 7.04 (d, J=8.6 Hz, 1H) 7.08-7.17 (m, 2H) 7.31 (dd, J=10.1, 2.4 Hz, 1H) 7.58 (d, J=8.6 Hz, 1H) 8.04 (br dd, J=7.3, 5.9 Hz, 1H).

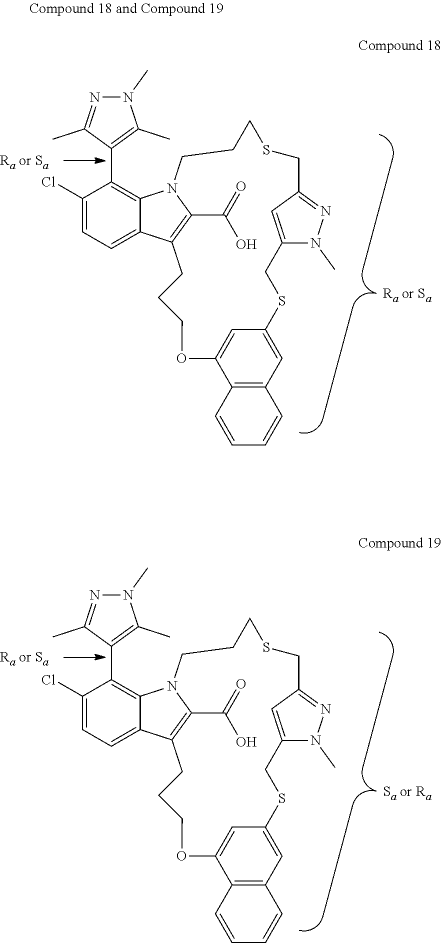

### Compound 18: Rₐ or Sₐ; Couple of Atropisomers but Absolute Stereochemistry Undetermined

### Compound 19: Sₐ or Rₐ; Couple of Atropisomers but Absolute Stereochemistry Undetermined

Compound 18 and Compound 19 are diastereoisomers of each other and are both mixtures of two stereoisomers.

A solution of Intermediate 84 (105 mg, 0.014 mmol) and LiOH (33 mg, 1.382 mmol, 10 eq.) in THE (3 mL), water (3 mL), and MeOH (3 mL) was stirred at 60° C. overnight. The reaction mixture was concentrated to the aqueous layer. Aqueous HCl (1 N) was added dropwise to the aqueous layer until the solution became cloudy. The mixture was extracted twice with EtOAc. The combined organic layer was dried by filtration on Extrelut NT3, and evaporated. The residue was purified by column chromatography on silica gel (Biotage Sfar 10 g; eluent: DCM/MeOH 100:0 to 0:100) to give Compound 18 (29 mg, yield: 30%) as a light brown solid, and Compound 19 (25 mg, yield: 26%) as an off-white solid.

Compound 18

¹H NMR (400 MHz, CHLOROFORM-d) δ ppm 1.00-1.19 (m, 2H) 1.72-1.81 (m, 2H) 1.98 (s, 7H) 2.37-2.51 (m, 2H) 3.27-3.43 (m, 4H) 3.75 (s, 3H) 3.81 (s, 4H) 3.91 (br s, 1H) 3.96-4.05 (m, 1H) 4.15 (br s, 2H) 4.27-4.38 (m, 2H) 5.60 (s, 1H) 6.92 (s, 1H) 7.27-7.32 (m, 3H) 7.36 (td, J=7.5, 1.3 Hz, 1H) 7.54 (d, J=8.0 Hz, 1H) 7.66 (d, J=8.6 Hz, 1H) 7.89 (d, J=8.4 Hz, 1H).

Compound 19

¹H NMR (400 MHz, CHLOROFORM-d) δ ppm 1.19-1.33 (m, 3H) 1.76-1.89 (m, 2H) 1.98 (d, J=4.3 Hz, 6H) 2.43 (br t, J=5.9 Hz, 2H) 3.16-3.29 (m, 2H) 3.31-3.46 (m, 2H) 3.59 (s, 3H) 3.81 (s, 4H) 3.86-3.98 (m, 3H) 3.98-4.16 (m, 5H) 4.17-4.36 (m, 2H) 5.48 (s, 1H) 6.39-6.43 (m, 1H) 7.08 (d, J=8.6 Hz, 1H) 7.35-7.42 (m, 2H) 7.42-7.48 (m, 2H) 7.54 (d, J=8.6 Hz, 1H) 7.65 (d, J=7.9 Hz, 1H) 7.97 (br d, J=8.3 Hz, 1H)

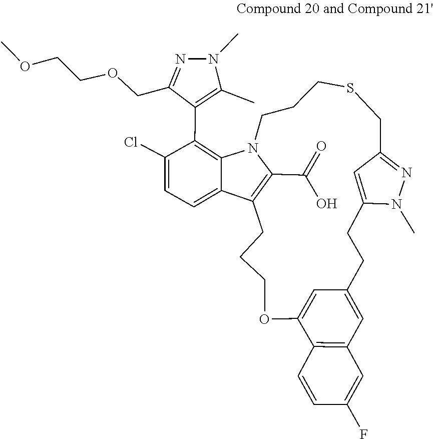

### Compound 20: Rₐ or Sₐ; One Atropisomer but Absolute Stereochemistry Undetermined

### Compound 21: Sₐ or Rₐ; One Atropisomer but Absolute Stereochemistry Undetermined

Intermediate 79 (130 mg, 0.178 mmol) was dissolved in dry THE (1.5 mL) and this solution was cooled to 0° C. under nitrogen atmosphere before NaH (60% dispersion in mineral oil, 9 mg, 0.214 mmol, 1.2 eq.) was added. The reaction mixture was stirred at 0° C. for 30 min. 2-Bromoethyl methyl ether (33 μL, 0.356 mmol, 2 eq.) was then added in one portion and the solution was stirred at room temperature for 16 h. The reaction was then quenched by addition of water and the solution was concentrated till dryness. The residue was purified by preparative SFC (Stationary phase: Chiralpak Diacel AD 20×250 mm, Mobile phase: CO₂, EtOH+0.4% iPrNH₂), followed by preparative HPLC (Stationary phase: RP XBridge Prep C18 OBD-10 μm, 50×150 mm, Mobile phase: 0.25% NH₄HCO₃ solution in water, CH₃CN) to give Compound 20 (16 mg, yield: 12%) and Compound 21 (16 mg, yield: 12%).

Compound 20

¹H NMR (400 MHz, CHLOROFORM-d, 27° C.) δ ppm 1.09-1.21 (m, 1H) 1.23-1.35 (m, 1H) 1.69-1.82 (m, 1H) 1.87-1.94 (m, 1H) 2.08 (s, 3H) 2.33-2.46 (m, 2H) 2.79-2.89 (m, 1H) 2.90-3.04 (m, 3H) 3.18 (br s, 3H) 3.21 (s, 3H) 3.23-3.28 (m, 1H) 3.34 (d, J=17.2 Hz, 5H) 3.40-3.50 (m, 2H) 3.78-3.91 (m, 3H) 3.85 (s, 3H) 3.92-3.99 (m, 1H) 4.27 (br d, J=2.6 Hz, 2H) 4.45 (br s, 1H) 5.66 (s, 1H) 6.03 (s, 1H) 7.02 (d, J=8.6 Hz, 1H) 7.13 (td, J=8.8, 2.4 Hz, 1H) 7.10 (br s, 1H) 7.29 (dd, J=10.1, 2.4 Hz, 1H) 7.56 (d, J=8.6 Hz, 1H) 8.03 (br dd, J=9.1, 5.8 Hz, 1H).

Compound 21

¹H NMR (400 MHz, CHLOROFORM-d, 27° C.) δ ppm 1.07-1.18 (m, 1H) 1.22-1.36 (m, 1H) 1.71-1.81 (m, 1H) 1.87-1.96 (m, 1H) 2.07 (s, 3H) 2.35-2.44 (m, 2H) 2.82-2.92 (m, 1H) 2.93-3.04 (m, 3H) 3.21 (s, 6H) 3.24-3.29 (m, 1H) 3.30-3.39 (m, 5H) 3.40-3.50 (m, 2H) 3.80-3.91 (m, 3H) 3.86 (s, 3H) 3.93-4.02 (m, 11H) 4.27 (s, 2H) 4.43 (br s, 1H) 5.67 (s, 1H) 6.05 (s, 1H) 7.05 (d, J=8.6 Hz, 1H) 7.10 (td, J=8.8, 2.9 Hz, 1H) 7.10 (br s, 1H) 7.29 (td, J=9.2, 2.4 Hz, 1H) 7.57 (d, J=8.6 Hz, 1H) 7.99 (br dd, J=8.9, 5.8 Hz, 1H).

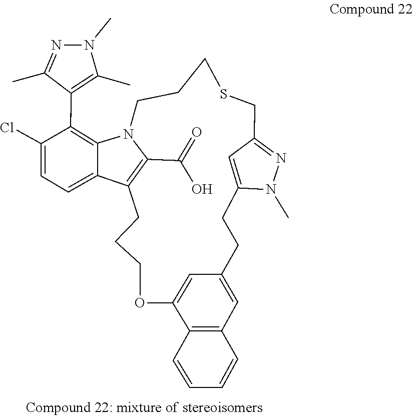 [SMILES: Cc1nn(C)c(C)c1-c1c(Cl)ccc2c3c(C(=O)O)n(c12)CCCSCc1cc(n(C)n1)CCc1cc(c2ccccc2c1)OCCC3]

Compound 22 was prepared according to an analogous procedure as for Compound 4, starting from Intermediate 87 instead of Intermediate 19.

¹H NMR (400 MHz, CHLOROFORM-d) δ ppm 0.83-1.03 (m, 2H) 1.71-1.89 (m, 2H) 1.91 (d, J=2.7 Hz, 6H) 2.34-2.46 (m, 2H) 2.84-3.05 (m, 4H) 3.26-3.44 (m, 7H) 3.77 (s, 3H) 3.85-4.01 (m, 3H) 4.41-4.94 (m, 2H) 5.66 (s, 1H) 6.07 (s, 1H) 7.08-7.15 (m, 2H) 7.28 (d, J=7.3 Hz, 1H) 7.34-7.41 (m, 1H) 7.58-7.66 (m, 2H) 7.69-7.77 (m, 1H).

Analytical Analysis

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, C₁), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min; column temperature (T) in ° C.; Run time in minutes)

LC-MS Methods:

LC-MS methods

| Method | | | | | Flow | Run |
|---|---|---|---|---|---|---|
| Code | Instrument | column | mobile phase | gradient | Col T | time |
| 1 | Waters: | Waters: | A: 10 mM | From 95% A | 0.8 | 2 |
| | Acquity ® | BEH C18 | CH₃COONH₄ | to 5% A in | 55 | |
| | UPLC ® - | (1.7 μm, | in 95% H₂O + | 1.3 min, | | |
| | DAD and | 2.1 × 50 | 5% CH₃CN | held for 0.7 | | |
| | SQD | mm) | B: CH₃CN | min | | |
| 2 | Waters: | Waters: | A: 10 mM | From 100% A | 0.6 | 3.5 |
| | Acquity ® | BEH (1.8 | CH₃COONH₄ | to 5% A in | 55 | |
| | UPLC ® - | μm, 2.1 * | in 95% H₂O + | 2.10 min, to 0% | | |
| | DAD and | 100 mm) | 5% CH₃CN | A in 0.90 | | |
| | SQD | | B: CH₃CN | min, to 5% A | | |
| | | | | in 0.5 min | | |
| 3 | Waters: | Waters: | A: 0.1% | From 100% A | 0.6 | 3.5 |
| | Acquity ® | BEH (1.8 | NH₄HCO₃ | to 5% A in | 55 | |
| | UPLC ® - | μm, 2.1 * | in 95% H₂O + | 2.10 min, to 0% | | |
| | DAD and | 100 mm) | 5% CH₃CN | A in 0.9 min | | |
| | SQD2 | | B: CH₃CN | to 5% A in 0.5 | | |
| | | | | min | | |
| 4 | Waters: | Waters: | A: 10 mM | From 100% A | 0.6 | 3.5 |
| | Acquity ® | BEH (1.8 | CH₃COONH₄ | to 5% A in | 55 | |
| | UPLC ® - | μm, 2.1 * | in 95% H₂O + | 2.10 min, to 0% | | |
| | DAD and | 100 mm) | 5% CH₃CN | A in 0.9 | | |
| | SQD | | B: CH₃CN | min, to 5% A | | |
| | | | | in 0.5 min | | |

LCMS results (RT means retention time)

| Com- | |
|---|---|
| pound | |
| num- | |
| ber | LCMS results |
| 1 | confirms the MW (RT: 1.05, [M + H]+ 700, LCMS Method 1) |
| 2 | confirms the MW (RT: 1.08, [M + H]+ 700, LCMS Method 1) |
| 3 | confirms the MW (RT: 1.08, [M + H]+ 700, LCMS Method 1) |
| 4 | confirms the MW (RT: 1.03, [M + H]+ 732, LCMS Method 1) |
| 5 | confirms the MW (RT: 1.03, [M + H]+ 732, LCMS Method 1) |
| 6 | confirms the MW (RT: 1.91, [M + H]+ 686, LCMS Method 2) |
| 7 | confirms the MW (RT: 1.92, [M + H]+ 686, LCMS Method 2) |
| 8 | confirms the MW (RT: 1.04, [M + H]+ 672, LCMS Method 1) |
| 9 | confirms the MW (RT: 1.00, [M + H]+ 744, LCMS Method 1) |
| 10 | confirms the MW (RT: 1.08, [M + H]+ 744, LCMS Method 1) |
| 11 | confirms the MW (RT: 1.85, [M + H]+ 668, LCMS Method 3) |
| 12 | confirms the MW (RT: 1.79, [M + H]+ 668, LCMS Method 3) |
| 13 | confirms the MW (RT: 1.83, [M + H]+ 686, LCMS Method 3) |
| 14 | confirms the MW (RT: 0.76, [M + H]+ 686, LCMS Method 1) |
| 15 | confirms the MW (RT: 0.94, [M + H]+ 686, LCMS Method 1) |
| 16 | confirms the MW (RT: 0.98, [M + H]+ 716, LCMS Method 1) |
| 17 | confirms the MW (RT: 0.98, [M + H]+ 716, LCMS Method 1) |
| 18 | confirms the MW (RT: 1.03, [M + H]+ 700, LCMS Method 1) |
| 19 | confirms the MW (RT: 1.05, [M + H]+ 700, LCMS Method 1) |
| 20 | confirms the MW (RT: 1.95, [M + H]+ 774, LCMS Method 4) |
| 21 | confirms the MW (RT: 1.95, [M + H]+ 774, LCMS Method 4) |
| 22 | confirms the MW (RT: 1.03, [M + H]+ 682, LCMS Method 1) |

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Analytical SFC-MS Methods (Flow expressed in mL/min; column temperature (Col T) in ° C.; Run time in minutes, Backpressure (BPR) in bars. “iPrNH₂” means isopropylamine, “iPrOH” means 2-propanol, “EtOH” means ethanol, “min” mean minutes, “DEA” means diethylamine.

| | | | | | Run |
|---|---|---|---|---|---|
| Method | | mobile | | Flow | time |
| code | column | phase | gradient | Col T | BPR |
| 1 | Daicel | A: CO₂ | 10%-50% B | 2.5 | 9.5 |
| | Chiralpak ® IH3 | B: iPrOH + | in 6 min, | 40 | 130 |
| | column (3.0 μm, | 0.2% | hold 3.5 min | | |
| | 150 × 4.6 mm) | iPrNH₂ | | | |
| 2 | Daicel | A: CO₂ | 10%-50% B | 2.5 | 9.5 |
| | Chiralpak ® IH3 | B: EtOH + | in 6 min, | 40 | 130 |
| | column (3.0 μm, | 0.2% | hold 3.5 min | | |
| | 150 × 4.6 mm) | iPrNH₂ | | | |
| 3 | Daicel | A: CO₂ | 10%-50% B | 2.5 | 9.5 |
| | Chiralpak ® IG3 | B: EtOH + | in 6 min, | 40 | 130 |
| | column (3.0 μm, | 0.2% | hold 3.5 min | | |
| | 150 × 4.6 mm) | iPrNH₂ | | | |
| 4 | Daicel | A: CO₂ | 10%-50% B | 2.5 | 9.5 |
| | Chiralpak ® AD-H | B: | in 6 min, | 40 | 110 |
| | column (3.0 μm, | iPrOH + | hold 3.5 min | | |
| | 150 × 4.6 mm) | 0.2% | | | |
| | | iPrNH₂ | | | |
| 5 | Daicel | A: CO₂ | 10%-50% B | 2.5 | 9.5 |
| | Chiralpak ® AD-H | B: EtOH + | in 6 min, | 40 | 110 |
| | column (3.0 μm, | 0.2% | hold 3.5 min | | |
| | 150 × 4.6 mm) | iPrNH₂ | | | |

TABLE
Analytical SFC data - Rₜ means retention time (in minutes),
[M + H]⁺ means the protonated mass of the
compound, method refers to the method used for (SFC)MS
analysis of enantiomerically pure compounds. No. means number.

| Compound No. | SFC Method | Rt | [M + H]⁺ |
|---|---|---|---|
| 2 | 1 | 5.56 | 700 |
| 3 | 1 | 4.37 | 700 |
| 4 | 1 | 5.45 | 732 |
| 5 | 1 | 4.65 | 732 |
| 6 | 2 | 4.01 | 686 |
| 7 | 2 | 4.38 | 686 |
| | 3 | 5.74 | 668 |
| 12 | 3 | 6.14 | 668 |
| 13 | 2 | 4.60 | 686 |
| 14 | 2 | 4.84 | 686 |
| 18 | 4 | 5.54 (44%) | 700 |
| | | and 5.69 | |
| | | (56%) | |
| 19 | 4 | 5.17 (44%) | 700 |
| | | and 5.35 | |
| | | (56%) | |
| 20 | 5 | 3.80 | 774 |
| 21 | 5 | 4.59 | 774 |

NMR

¹H NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

Pharmacological Analysis

### Biological Example 1

Terbium labeled Myeloid Cell Leukemia 1 (Mcl-1) homogeneous time-resolved fluorescence (HTRF) binding assay utilizing the BIM BH3 peptide (H₂N—(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1× assay buffer, where the following ingredients were added fresh to stock buffer: 2 mM dithiothreitol (DTT), 0.0025% Tween-20, 0.1 mg/mL bovine serum albumin (BSA). The 1×Tb-Mcl-1+Cy5 Bim peptide solution was prepared by diluting the protein stock solution using the 1× assay buffer (b) to 25 pM Tb-Mcl-1 and 8 nM Cy5 Bim peptide.

Using the Acoustic ECHO, 100 nL of 100× test compound(s) were dispensed into individual wells of a white 384-well Perkin Elmer Proxiplate, for a final compound concentration of 1× and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1×Tb-Mcl-1+Cy5 Bim peptide solution. The plate was centrifuged with a cover plate at 1000 rpm for 1 minute, then incubated for 60 minutes at room temperature with plates covered.

The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition=(((NC−IC)−(compound−IC))/(NC−IC))*100

where IC (inhibitor control, low signal)=mean signal of 1×Tb-MCl-1+Cy5 Bim peptide+ inhibitor control or 100% inhibition of Mcl-1; NC (neutral control, high signal)=mean signal 1×Tb-MCl-1+Cy5 Bim peptide with DMSO only or 0% inhibition

An 11-point dose response curve was generated to determine IC₅₀ values (using GenData) based on the following equation:

Y=Bottom+(Top−Bottom)/(1+10\hat{ }((log IC₅₀−X)*HillSlope))

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

Kᵢ=IC₅₀/(1+[L]/K_{d})

In this assay [L]=8 nM and K_{d}=10 nM

Representative compounds of the present invention were tested according to the procedure as described above, with results as listed in the Table below (n.d. means not determined).

| Compound | HTRF Kᵢ (nM) |
|---|---|
| 1 | 0.126 |
| 2 | 0.084 |
| 3 | 0.133 |
| 4 | 0.251 |
| 5 | 0.276 |
| 6 | 0.056 |
| 7 | 0.086 |
| 8 | 0.977 |
| 9 | 0.139 |
| 10 | 0.093 |
| 11 | 0.307 |
| 12 | 0.396 |
| 13 | 0.392 |
| 14 | 0.423 |
| 15 | 0.426 |
| 16 | 0.056 |
| 17 | 0.080 |
| 18 | 1.440 |
| 19 | 0.249 |
| 20 | 0.073 |
| 21 | 0.155 |
| 22 | NT |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and -7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

Materials:
Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| | | |
|---|---|---|
| Product | Units | Storage |

| | | | | |
|---|---|---|---|---|
| RPMI1640 (no L-Glutamine, no | 500 | mL | 4° | C. |
| phenol red) | | | | |
| Foetal Bovine Serum (FBS) (Heat | 500 | mL | 4° | C. |
| inactivated) | | | | |
| L-Glutamine (200 mM) | 100 | ml | −20° | C. |
| Gentamicin (50 mg/mL) | 100 | mL | 4° | C. |
| Caspase 3/7 Detection kit | 100 | mL | −20° | C. |

| | |
|---|---|
| 10 × 10 | mL |

Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

Caspase-Glo Reagent:

The assay reagent was prepared by transferring the buffer solution to the substrate vial and mixing. The solution may be stored for up to 1 week at 4° C. with negligible loss of signal.

Assay Procedure:

Compounds were delivered in assay-ready plates (Proxiplate) and stored at −20° C.

Assays always include 1 reference compound plate containing reference compounds. The plates were spotted with 40 nL of compounds (0.5% DMSO final in cells; serial dilution; 30 μM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 μM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

LC=median of the Low Control values

=Central Reference in Screener

=DMSO

=0%

HC=Median of the High Control values

=Scale Reference in Screener

=30 μM of positive control

=100% apoptosis induction

% Effect (AC₅₀)=100−((sample−LC)/(HC−LC))*100

% Control=(sample/HC)*100

% Control min=((sample−LC)/(HC−LC))*100

TABLE
Measured AC₅₀ for Representative Compounds of Formula
(I). Averaged values are reported over all runs
on all batches of a particular compound.

| Compound | MOLP8 Caspase-Glo AC₅₀ (nM) |
|---|---|
| 1 | 90.5 |
| 2 | 129.9 |
| 3 | 207.4 |
| 4 | 7029.1 |
| 5 | 7274.4 |
| 6 | 236.6 |
| 7 | 231.1 |
| 8 | 1340.6 |
| 9 | 232.2 |
| 10 | 160.2 |
| 11 | 1455.1 |
| 12 | 1349.6 |
| 13 | 1343.4 |
| 14 | 1536.7 |
| 15 | NT |
| 16 | 414.4 |
| 17 | 1829.8 |
| 18 | 3645.0 |
| 19 | 740.5 |
| 20 | 173.1 |
| 21 | 1010.4 |
| 22 | NT |

1. A compound of Formula (I) 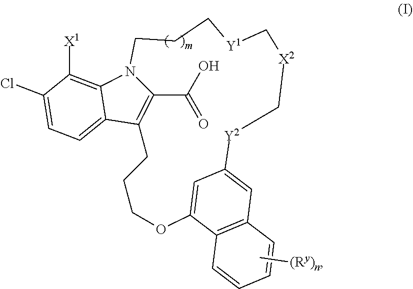
or a tautomer or a stereoisomeric form thereof, wherein
X¹ represents 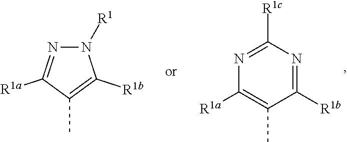
wherein
R¹, R^{1a}, R^{1b}, R^{1c}, and R² each independently represent hydrogen; or C₁₋₆alkyl optionally substituted with one or two substituents each independently selected from the group consisting of Het¹, —OR³, and —NR^{4a}R^{4b};
Het¹ represents morpholinyl or tetrahydropyranyl;
R³ represents hydrogen, C₁₋₄alkyl, —C₂₋₄alkyl-O—C₁₋₄alkyl, —C₂₋₄alkyl-OH, or —C₂₋₄alkyl-O—C₂₋₄alkyl-O—C₁₋₄alkyl;
R^{4a} and R^{4b} are each independently selected from the group consisting of hydrogen and C₁₋₄alkyl;
X² represents 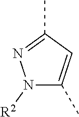
which can be attached to the remainder of the molecule in both directions;
Y¹ represents —O—, —S—, —S(═O)—, —S(═O)₂—, or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
Y² represents —CH₂—, —S— or —S(═O)₂—;
R^{y} represents halo;
n represents 0, 1 or 2;
m represents 0 or 1;
or a pharmaceutically acceptable salt, or a solvate thereof.

2. The compound according to claim 1, wherein
R¹, R^{1a}, R^{1b}, R^{1c}, and R² each independently represent hydrogen; or C₁₋₆alkyl optionally substituted with one —OR³;
R³ represents hydrogen, C₁₋₄alkyl, or —C₂₋₄alkyl-O—C₁₋₄alkyl;
Y¹ represents —S—, —S(═O)—, or —S(═O)₂—;
Y² represents —CH₂— or —S—;
n represents 0 or 1.

3. The compound according to claim 1, wherein Formula (I) is limited to Formula (I-y)

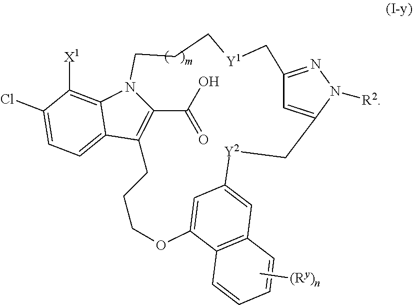

4. The compound according to claim 1, wherein
X¹ represents

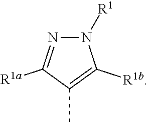

5. The compound according to claim 1, wherein
X¹ represents

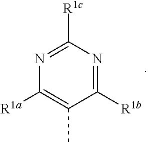

6. The compound according to claim 1, wherein
n represent 1; and R^{y} represents fluoro.

7. The compound according to claim 1, wherein
Y¹ represents —S—, —S(═O)—, or —S(═O)₂—.

8. The compound according to claim 1, wherein
m represents 0.

9. The compound according to claim 1, wherein
m represents 1.

10. A pharmaceutical composition comprising a compound as claimed in claim 1 and a pharmaceutically acceptable carrier or diluent.

11. A process for preparing a pharmaceutical composition comprising mixing a pharmaceutically acceptable carrier with a therapeutically effective amount of a compound according to claim 1.

12-14. (canceled)

15. A method of treating cancer, comprising administering to a subject in need thereof, a therapeutically effective amount of a compound as claimed in claim 1.

16. The method according to claim 15, wherein cancer is selected from prostate, lung, pancreatic, breast, ovarian, cervical, melanoma, B-cell chronic lymphocytic leukemia (CLL), acute myeloid leukemia (AML), and acute lymphoblastic leukemia (ALL).
